SEQUENCE LISTING (128 sequences)

SEQ ID NO: 1 - DNA, 1188 bp, Lactuca sativa
atggcttttcaatctaaactcatatctatctctacctcttttctgattttgatctctactatattcttccatttcaccttattatcctcatcgtatatgctagttcatcatatcaacaacaacaatgaacataactctcaccatcgacgccctatcctccaagccaatcagtctcaatgttctctcttcatgggttcatgggttcacgacgattcctacccgatctaccagtcatcttcttgccccgttatcgatcctgatttcaattgccaaatgtacggtcgacccgactccgattacctcaagtaccgctggaaaccggccaactgtgaactccccaggttcaacgggctcgagtttctggaaaagatgaaagggaagacagtaatgttcgtgggggactctctggggaggaaccaatgggagtcgttgatttgtctgacgtggtcatcggtgccacgtgttgccacccaagtgatccgagcatatccgctctcaacgttcaagtttttggattacggtgtaacgttgtcgtattacaaagcacagtatcttgtcgacattgattctgtcgggggacgaagggtactcaagctgcaggacatatcggaaaacgcccaggcatggcgggacgtcgacgtattcgtgttcaacaccggtcactggtggagccacaccggcgccacccaagggtgggacatgatggaatccggtgggtcgctttaccaggatatggatcgtatggtcgccatggaaaccgccgtaaggacgtgggcccgatgggttgattcgaatatcgacaccactaaaaccagagtctttttccaatcgttctcacctactcactacgatccgtcggaatggaatggaggatcggggtcgacaaccaccgcgaagagctgctatggggagacggctccggtgagtgcttctgcgttcttgggggtgtacccgaacgagatgaaggtggtggacgccgtgctccgggaaatgcaaacgccggtttacatgcttgacatcacgactctttcagctatgaggaaagatgctcatccatcgatttacagtggggatacgagcacgggtcaaaaggctgaccctaatggcacccctgattgtacacattggtgtcttcctgggttaccggatacttggaaccaactcttctacacggctttgttcttttag

SEQ ID NO: 2 - DNA, 1534 bp, Solanum lycopersicum
atcctttctttcacttttgctcttttcttttcttctctttgttgttttcaattacttccaatgaatctcttctcttctctaaaattttcatcttattcttcttcaactaaatcattttcctttagatttctattatgtctttttttactaaattttttttatgtacttacatttgcaagtctaaggaaccatcaatatcaacaaaatcataaaaaaccaattatgattttggcaaatttaacatcatgttctttgtttatgggtagttgggtttatgatgaaacttatccattgtatcaatcttcatcttgcccaattattgattcacaatttaattgtcaaatgtatggaagaccagacactgattacctcaaatatagatggaaaccaacaaattgtgaaattccaaggttcaatggtctagattttttgttgagaatgagagggaagacaataatgtatgttggagattcattaggaagaaatcaatggcagtcattgatttgtatgatttcagcagctgtcccacgtggagctcaaacacaattcatcacgggtgaccctctatctacttacaaattcttggattatggagtgtcagtgtcattttatagaacaccatatttagtagacatagattcagtgcaaggcaagagaattctgaaattagatgatataactaagaattctattgcttggagaggtgttgatgtcttgtctttcaatactggccattggtggactcacaaaggggctcttcaagggtgggactacatggatttaggaggcaaattgtatcaagatatggataggctagtggcattagagagaggcttgaggacatgggcaagatggattgatgccaatattgacaaaagtagaacaaaactcttctttcagggcatttcacctacacattacaacccaagcgattggaccaccggcacgtcgacctcgtcatcaacgaggacgtgttacggtgagaccgtgccgatgaccggcacggcgtatccgggcacgttcccggatcaaatgggggtgttgaaggaggtaataggagacatgaacaaccctccatttattttggacataacatttttatcagctatgagaaaagatgcacacccttctatctatagtggtgcactcacttctgagcaaaaggcaaatcctgatcactctgctgattgtagtcattggtgtctagctggattgccagatacttggaatcaactattttatactgctcttttcttctagatcgttagtatttatcacgatatcagattcagctaagcaagtatgaaattcttaatttgctcgatacgacatggtggcaattggtgcaaagttatatttagggaacacatatttcacatctactaagatcaaagatgtacttgagtagagttttagtttctattggtgtaaatgtgtttttaagaaaaagtatgttgttcattttgtaattattcactttttgttggtccattttattgaactttggttatatgatcccatatcaaaaaaaaaa

SEQ ID NO: 3 - DNA, 1173 bp, Cucumis sativus
atggggtttttgggattttggccttgtttggcagcattgtttttcatttctcttcatacccatttagcttcctcagctttgattctcagtttgaagcaccattaccggaacagcgaccaccataatcgccgcccaatgcttcaagctaaccaaaccggctgtgctctgttcgccggaacttgggttcgagatgattcatatccactttaccaagcttctaactgtccatttatcgatcctgagttcaactgccaagcttacggcagacccgattcaaattacctcaagtaccgttggcagccgctcgattgtgagctcccaaggttcgatggggctgagtttttgatgagaatgagaggaagaactgtgatgtttgttggtgattcattggggagaaaccaatgggagtcattgatttgtttgatcgtgtcatcttctcctcaaactcctactcaaatgactagaggagaacctctttcaaccttcagattcctggaatatgagttaactgtgtcctattacaaagccccgtatcttgtggacatagagatagagaatgggaagagagtgttgaagctggaggagatatcaatgaatggaaatgcttgggttggagctgatgttatttccttcaacactggacattggtggagccacactggctctctacaagggtgggattacatggaatcaggaggatcatactatcaagacatggatcggttgggtgcaatggaaaaggctttaagaacatgggctgattgggttgagaagaacattgatgtctctagaacaagggttttcttccaagctatctcccccacacattacaatccatctgaatggaacacggggacagcatcgatgatgacatcaacgaaaaattgttatggggaaacggcaccaatgggggggacgacgtacccgggagggtaccctattcaaatgagggttgtggatgaagtgataagggagatgaggaagccagtatacttattggacataacaatgttatctgagctaagaaaagatggacacccttccatttatagtggtgatttgaatcctcaacaaagggctaacccagatagatcagcggattgtagccattggtgtcttcctggcttaccagatacttggaaccaattgttttatactgcattgttcttttag

SEQ ID NO: 4 - DNA, 1469 bp, Hordeum vulgare
gctaataattctctcttcgtcacgagccgcctcgcgctccgaccgtcgctaacccaccgcagcaagagagttaatcgctgctagcttgcctcgtgatcgattcgtcggccggtggtacaaaagcgccgggggaaaggtggcctcgcctttccctctccttgtctttgtggaagatgctgctcttgtgcaagagctccgtgctcgccgtgctgctgctccacgccgtctcgctgtccgcctcggcgctcgccgtcgggctcgcgcggcgccaccggcgcgacgtcctgcccggccccaagggctgcgacgtcttcagcggcagctgggtccgcgatgtcgacggctccgcggcggagggctacaccgggtacaagtgcccggtcatcgacgcggagttcaactgccagctctatggccgcccggactccgactacctccggtaccgctggaagccggccagctgcgagctacccaggtttgacggcgccgactttttgacgcggatgaaggggaagacggtgatgttcgtgggggactcgctgggccgtaaccagtgggagtcgctcgtctgcctgctgcacgccgccgcgccgcagtcgccgtcgcagctcgtctccgccgaccctctgtacacctacaggttcatggagtacgagctggtggtgtctttctaccgcgcgccgtacctggtggacatcgacgtggtgcaggggaaacgggtcctgatgctcgactacatcgccgagaacgcccaagcgtggcgcggcgccgacgtgctctccttcaactccggccactggtggacgcacaccggcgcgctccaggggtgggattacatgggggagggcgggcgctactcggaggacatggatcggatggtggcgttccagcgcgggatgaccacatgggccaactgggtggacctcaacgtcgacccggccaagacccgtgtcttcttccagtccatgtcccccacccactacagctccaaggagtggccgaatccggtgtccaagaactgctacggggagacggtcccgttggctgggcccaataacacgtcgcagccgatgggccaggagcaggtgacgaaaacggtgctgcaggggatgaaaagcccggtccgtttgctcgacatcacggcgctgtcggcgctgcggaaggacgcgcacccgtcagtttacagcggcgacttctcgccggcgcagcgcggcaaccccgccggctccgccgactgcagccactggtgcctcccgggcctacccgacacctggaaccagctcttctacaccttgctcttctaccaatagcaaccgtactggactattgctgcatttggccagatgctgcagatgcagtgctggtattaatcgtttccctctgattttcgctgtggatgcgttgtgcgagcagtatatgcagacggggttgatgaggttgtacaacctctgaatgccaacctgtagccc

SEQ ID NO: 5 - DNA, 1134 bp, Triticum aestivum
atgctgctcttgtgcaagagctccgtgctcgccgtcctgctcctccacgccctctctctgcccgcctcggcgctcgccgtcgggctcgggcggcgccaccgccgcgacgtcctgcccgggcccaagggctgcgacgtcttcagcggcagctgggtccgcgacgacggctccgccgaggctgcctacaccggctacaagtgcccggtcatcgacgcggagttcaactgccagctctacggccggccggactccgactacctccggtaccgctggaagccggccagctgcgagctacccaggtttgacggcgcagactttttgacgcggatgaaggggaagacggtgatgttcgtgggggattcgctgggccgtaaccagtgggagtcgctcgtctgcctgctgcacgccgccgcaccgcagtcgccgtcgcagctcgtctccgccgaccctctctatacctacaggttcatggagtacgagctggtggtgtctttctaccgcgcgccgtacctggtggacatcgacgtggtgcaggggaaacgggtcctcatgctcgacgacatcgccgagaacgcccaggcatggcgcggcgccgacgtgctctccttcaactccggccactggtggacgcacaccggcgcgcttcaggggtgggattacatgggggagggcgggcggtactcggaggacatggataggatggtggcgttccagcgcgggatgaccacatgggccaactgggtggacctcaacgtcgacccggccaagacccgtgtcttcttccagtccatgtcccccacccactacagctccaaggagtggcccaacccggtgtccaagaactgctacggagagacggtcccattggctgggctcaactccacggcgcagccgatgggccaggagcaggtgacgaaaacggtgctgcagggcatgaaaagcccggtccgtttgctcgacatcacggcgctgtcggcgctgcggaaggacgcgcacccgtcggtttacagcggcgacttctcgccggcgcagcgcggcaaccccgccggctccgccgactgcagccactggtgcctcccgggcctaccggacacctggaaccagctcttctacaccttgctcttctaccaatag

SEQ ID NO: 6 - DNA, 1179 bp, Cucumis sp.
atgggctttttgggattttggccttatttggcagcattgtttttcatttctcttcatacccatttagcttcctcagctttgattctcagtttgaagcaccattaccggaacagcgaccaccataatcgccgcccgatgcttcaagctaaccaaaccagctgtgctctgttcgccggaacttgggttcgagataattcatatccactttaccaagcttctaattgtccatttatcgatcctgagttcaactgccaagcttacggccgaccggattcaaattacctccaataccgttggcagccgctcgattgtgagctcccaaggttcgatggggctgagtttttgatgagaatgagaggaagaacagtgatgtttgttggtgattcattggggagaaaccaatgggagtcattgatttgtttgatcttctcatcttctcctcaaactcctactcaaatgactagaggagaacctctttcaaccttcagattcctggaatatgagttaactgtgtcctattacaaagccccatatcttgtggacatagagatagagaatgggaagagagtgttaaagctggaggagatatcaatgaacggaaatgcttgggttggagctgatgtgatttccttcaacactggacattggtggagccacactggctctttacaggggtgggattacatggaatctggaggatcatattatcaagacatggatcggttgggtgcaatggaaaaggctctaagaacatgggctgattgggttgagaagaacattgatgtctctagaacaagggttttcttccaagctatctcccccacacattacaatccatctgaatggaacacagggacagggacagcatcaatgatgacatcaacgaagaattgttatggggaaacggcacccatgggggggacgacgtacccgggagcgtaccctattcaaatgagggttgtggatgaagtgataagggagatgaggaagccagtatacttattggacataacaatgttgtctgagctaagaaaagatggacatccttccatttatagtggtgatttgaatcctcaacaaagggctaacccagatagatcagctgattgtagccattggtgtcttcctggcttacctgatacttggaaccaattgttttatactgcattgttcttttag

SEQ ID NO: 7 - DNA, 1941 bp, Zea mays
aactgaacatgggataagaataaaccaacaaaacacatgtgttttcgtggtatatactctatatactcagatataaatggaaaccgtaataacatatggccatctatctagttggccctttgaaaacgagatatgcaaagtatgttccatttccaagaaagcacgaattccatggctgcgaatctttggatgttcctacaccgacgtacgccgaagggcaaaccgggcaaggcaacgaatccacggattccatacgggtgcaaactacttacgagatctcgaacacgctcacgtgaactccagtcggggtctagcggcaattcttaccctcacatgcacacctcctcttcaatcgaactcctgagaattagcgcagctagatagattactcgcggctatctggattctggactcgtggttacacgatgaaccgagcagcagcataaaggggccatcagaaagacggctttgcttccgttctcttctcttttgtttgccacgatgctgccgtcccgcaggaagagtgccgtcctcggcgtcgtcgcgttgtcctcctccgtcctcatcctgctccacgccttctctctcccggcagcctctgcgctctccgtcggcctggcgcggcgccacaggcacgatgccacgcccgggctggccgccggcggctgcgacgtcttcagcggcagctgggtcctcgacggcggtagctcggcctcggcggcgtacaccgggtacaactgcccgctcatcgacgcggagttcaactgccagctctacggccgcccggactccgactacctccggtacctctggaagccggccggctgcgagctaccgaggttcgatggcgcggacttcctgacgcggatgaaggggaagacggtgatgttcgtgggcgactcgctggggcgcaaccagtgggagtcgctcgtctgcctgctgcacgccgccgcgccgcagtcgccggcgcagctcgtctccgcggaccctttgtacacctacaagttcctggagtaccaggtgacggtatccttctaccgcgcgccgtacctggtggacatcgacgtggtccaggggaagcgggtgctgatgctggacgacatctccgagaacgccgagtcgtggcgcgacgccgacgtgctctccttcaactccggccactggtggacgcacaccggctcgatgcaggggtgggactacatgggcgagtctgggcgatactacgaggacatggaccgcacggtggcgttccagcgcggcctcaccacctgggccagctgggtggacctcaacctcgatcaagccaagacccgtgtcttcttccagtccatgtcgcccacgcactacagctcgaaggaatggcccaacccggtgtcgaagaactgctacggcgagacggcaccggcgaccgggctcaactcgaccggccaggcctcgggccaggaccaggtgatccaggccgtgctgcggggcatgaagagccccgtccgtctcctcgacatcacggcgctgtcggcgatgcggaaggacgcgcacccgtcggcatacagcggcgacttctcgccggcgcagcgcgccaaccccggcgccggctccgttgactgcagccactggtgccttcccggcctcccggacacctggaaccagctcttctacacactcctcttctacaaatagcagtgacaccgcgatcgacacagaagaaagtcatccaaatccaactgtacacctagcagctcgccagccattcgttttttttcgttgtgtttgtgtgtaaaactattgtgacgtcgttgcatcacaggagtatacacaattagttggcgggagaaatgcgtcgataacgtttaaataaaagtgaagaacgtacaatctcggcatcaattgaacgtacatggcatttgcacaattgtactggacgccccatttttcgcaatagat

SEQ ID NO: 8 - DNA, 1167 bp, Glycine max
atggcacttctccattccctatttgcccttctcttccttctcctttcccttcaactccatacagtctcatgtgccctcctactaagcctgagacaccaccacaacactctccaccagcagaggccaatgatccatgccaaccaaaccaactgtgcactttttgtgggaacttgggtccaagatgactcttaccctatctatcaatcctctaactgccccatcatagatccacagttcaactgcaagatgtttggccgccctgattctgattacctcagatacagatggagacccctcaactgtgacctccctaggttcaatggggtggagtttctactgcaaatgaagggcaaaactgtgatgtttgtgggtgactcactagggcgcaaccaatggcaatcactgatttgcatgatatatgctgcagttcctcagacacaaacacaattggtcagaggggaaccactctcaaccttcagattcttggactacggtgtgaccatttcattttacagggctccttatttggtagagattgatgtggtccaagggaagaggattttgaggttggaggaggttgatgggaatggtgacgtttggagaagtgttgatgtgctgtctttcaacactggacattggtgggatcatcaaggctctcttcaagggtgggattatatggaattaggaggcaaatactaccaagatatggatcgtttagcagctttggaaaggggtatgaaaacatgggctaactgggtggactccaatgttgatagaagcagaaccaaagtgttcttcctgggaatttctccttcacataccaacccaaatgaatggaattctggagtgacagcaggactgactacaaagaactgctatggtgaaactactccaattactagcactggcacagcataccctggtgtgtaccctgaacaaatgagggttgtggacatggtaattagaggaatgagtaaccctgcttatcttcttgacatcacaatgctgtcagcatttaggaaggatgcacatccctccatttatagtggtgatttgaatcctcaacaaagagctaaccctacctactcagctgattgtagccactggtgtcttcctggattgccagatacttggaatgaactattctatactaccttgttctattaa

SEQ ID NO: 9 - DNA, 1469 bp, Oryza sativa
caattctcatcgccatcgatgccgctcttctgcaagcacggcgccggctttcttgccgtcgccgtgtctacatccatcgtcttcctcctcctccttcacgccttctcgctgccggcctcggcgctctccttcgggctagcgcgccgccaccggaaagacgccgccgtgagctgcgacgtcttcagcggcagctgggtccgcgacgacggcggcggcggcgcggcggcgtacaccgggtacaactgcccggtcatcgacgcggagttcaactgccagctgtacggccggccggactccgactacctccggtaccggtggaagccggccggctgcgagctacccaggtttgatggcgcggacttcttgacgcggatgaaggggaagacggtgatgttcgtcggcgactcgctgggccggaaccagtgggagtcgctcatctgcctgctgcacgccgcggcgccgcagtcgccggcgcagctcgtctccgtcgaccctctctacacgtacaagttcctggagtacggtttggtggtatcgttctaccgcgcgccgtacctggtggacatcggcgtggctcaggggaagaaggtgctatggctggacgacatctccgggaacggcgaggcgtggcgcggcgccgacgtgctctccttcaactccggccactggtggacgcacaccggcacgctccaggggtgggattacatgggcgcggacgggcggtactacgaggacatggaccggatggtggcgttccagcgcgggatggcaacctgggccaactgggtggacgccaacgtcgatccagccaagacccgtgtcttctaccagtccatgtcccccacacactacagctcgaaggagtgggccaacccggtgtcgaagaactgctacggcgagacggccccggcggcggcggcggcggcggggctgaacgcgacgacggcgcaggcgacgtcggggcaggaccaggtgatacaggccacgctgcggtcgatgaagagccccgttcgcctcctcgacatctcggcgctgtcggcgctgcgcaaggacgcgcacccgtcggtgtacagcggcgacctctcgccggcgcagcgcgccaaccccggcggcggctcggcggactgcagccactggtgcctccccggcctgccggacacgtggaaccagctcttctatgcgcttctcttctaccaatagctgtagcctgtaggcagttaatttcgtttttttttatatcgctcggtgtaacaatcttcagagtgaaggtgaggaggcgtggtgacagtaggcacacatggatgagactctgagaagaatcaagaatgtacattgattgatcatgcgctgccaaattgcagttgcagtgtacacggttgtactatgatctgtctgtgatctgtcgctaaatactctgataattgtgactctggtacccacatctaaacatctttgataaccttgcacaa

SEQ ID NO: 10 - DNA, 1951 bp, Solanum lycopersicum
taaatatattatttttatattctatcatagtgaaaaaagaaaggtgatttccattataactcaataaaactcaatatgtcttgatttaaatactaagataatttatgttacttctttgactcaaaaatttaagggcaacccttagctattcaatttaatttaggttattagtttgctattttataccattttccaaaatttaatttgaaagattcgacaaggagacatacaatattaaaataggtatgatgttttgtttgttatagaagtaaagcaaagaacatttaaccattcacatatttccttttttcgttttttaaagtattttctttcctctattctttattggaatatgaaatttgatttattgtagcaagaaacataaattcctttacttataatgaagtttgaaatatatgtatcagacaagatatgacctccctcacattagcccttccccacctttactttctttcgtatatttaaggttaggtttcgtgaaaaaaaatattaattgttttcatatataataagttttataatgacgaaaaggagttggtaagcactgcactcacaaatcgaaaattgctaattcgaatcaccaacggagcaaaagaatgaaaacttctagaaagggctaaaaagaagtttataaaaatatcattataatataatttaaataaatattaaaaaaataaaaatgatagacattacgtattgaggatgaagggaggagcgatattgcaaagaggtggtgatgatggtgtgggttctgaagtccgagtaggatgtcacttattaaatctatttttatcgaaaataatttaagaaaaacatatttcatataaaaatattttttattgttcataattaatgatttttttcttcgaatggtccatacatcttctcctagagacatagttataatgtggtccacatgtgcaaaaaaattttcttctatgtctcaataaagaattattactcatctgtctccattcaagtatacgtagtttgaattagcatataaaaagttataatctataataaatatattaaatattaagatgaattaacttattaagaatataacatattataaattataagaataatattcatttttaaaatgaaattaaaaaaaaaatgtaacacataaattaaaaataaacataagtacattttttgagaagttagatctctcgaggctactaaatgacagagttgttccaaattctaaacttttttattgattaatttcttcgttaaattaagcatcaaaagttgtgaaaccactttcccgccaaaacctatataactattttcgactcaaaatttatacaataaacatatctatataaaaaaaattgaatataatatcataaataattgaatctacatacataagattgaaagagtttaatctgtcttgctttatgatttaatcctcatgttccgaaaaaaaaaatactcatcgaccatcaaagacgatgaattttttataagacccttgtacatgccgtctcctctccccatcttatacctaaattgaatttaacttctaatgataatgagtaaaacctgttgtaggcggctttttaactttctaacgcattaacttaaaattttgaatttaattatcagcgattcaatataccttaggtaaatataagtagataatatttaaatcttaaattcgtctctccgtgctccaccggaaaagtcagccatcttcacatgcaccctgcaacaaaatatctccatttctccatcacccataggcccatgcctcagttctttcaaaatagtacattcacaaagcgaccttctttttcccctctctttctcacccctttttaaagtctgtttctttcttctctctctatatctttttcactttattaatacactttttttttctctttccttaacccttttttaaatttctcccaaa

SEQ ID NO: 11 - DNA, 2001 bp, Glycine max
aaaagtaaaatcaatataattttttaatcttttaataattatagtgattctagttttatatccttttatactaatctttcttgcaacatcttctcattctcacttttgaatttaaacaatagatttaaaattatatattgattttttaattactacaattaaatttaatgacatatttaaatggattgtaataatatgcatatactaaagtaatgagggtaaaaaattgacggaaggattagtgaattatgtggatgaagaaattagagatgacaatcacaaaaataatgtgttgaacatgtggttagcataatctgtataagaaaaacatgactagtcttgggacataaaataaacattaatttaatttggaaaataaagggaaagagcatagaaggtgtatatttaacatatttttttattccaaaagaataggagaaatatatatgatcacacatcttgaaaaatatgaaagagagaagagggtatgtgtaatgagagaagaagaaagtttgacaaccaatagaaaaagaaaaaaaaagtctatcaaaatcttgtttgaatgagattgtttgatatatgttttatactaaaaatggagtgaaatccatcaaaatccttcttaaaaacaatccatccaaatttatatattttttaatattaaaagactttttataagttataaaaaatcttaatcgaatatcatcattttgttgtcttccttaacaaatcttaaaattcttaattaaataccataaaacttatttgtattatttaaaaatcacgattgaatatcacaagactttttttatataaaaagtcttttaaaatcctttaaaatcttaatccaatacacccctaaatttgatttgttatgatgaaatatgagaactttttattcaaccattccaattaattaattgtgtgaatgcatcaaatttccaactacagaaatcttgatcctcaaataccataatttcactgtcattttatttgagatttttttcccattctcttctagagaacaactgtttgtagaatgtaaagtaataaatttgaattgatgatgacaaaattaatgttcaatatcaagtatacatgtctttcaaaaaaaaaatattaagtatacacttataataaattatagatttattgaagaaaatagctaataaacattacttaacattgaacaaattcttttaaatacattttcttaaaaatatttttagtaaattcaaacaaaaataaaatgtgttgaaaaaataaatgactgagacaaaaatgcacacctaatctcttcattacacactagaatgagaaagaacacaataaataaaagtataacactataagaaaatatcacaattcatttgcttatcgtgattcaaacttttattcttaccttcaggatctcaaatttatttaatgtgatatcacaaacaaatattatttataaacttagattataatttataaataatatcttaaaatctaaatagcattctataaataacataatatatttttttaaaaatctaaacaatattatataaatttaaaacgaagaatttgaattatttccaacaactctcctcctatattaaaatatatgatccattttctcatcaacaactaaaaaattgcatgctcatacttttttcttatgagagggacgaacaaaagttcaaaggcatgtacaaaagaagtctaactgattaaacataatatatttacatcagtattgtaaacatcttcattctatctttaattcgattaaaaaaatacttctttttatttaccacaacttaccactttaatgagtctaccttctttacatgtacacaacaaaacatatctccaccattattctaacattattttcacagacaccaaaaatcctcatcctttatatattcacatcttttcaaaacagtccaaacatccttcttccctctcccccatccctttttgtttccaacatttcctttcctctctctccta

SEQ ID NO: 12 - DNA, 25 bp, Glycine max
tgtatctgaggtaatcagaatcagg

SEQ ID NO: 13 - DNA, 25 bp, Glycine max
gaggtcacagttgaggggtctccat

SEQ ID NO: 14 - DNA, 25 bp, Glycine max
agaaactccaccccattgaacctag

SEQ ID NO: 15 - DNA, 25 bp, Glycine max
catcacagttttgcccttcatttgc

SEQ ID NO: 16 - DNA, 25 bp, Glycine max
tgaagagagccttgatgatcccacc

SEQ ID NO: 17 - DNA, 25 bp, Glycine max
cctcctaattccatataatcccacc

SEQ ID NO: 18 - DNA, 25 bp, Glycine max
agtttcaccatagcagttctttgta

SEQ ID NO: 19 - DNA, 25 bp, Glycine max
gctgtgccagtgctagtaattggag

SEQ ID NO: 20 - DNA, 25 bp, Aqueoria victoria
gttgtagttgtactccatcttattg

SEQ ID NO: 21 - DNA, 25 bp, Hordeum vulgare
gcggagccgtcgacatcgcggaccc

SEQ ID NO: 22 - DNA, 25 bp, Hordeum vulgare
cacttgtacccggtgtagccctccg

SEQ ID NO: 23 - DNA, 25 bp, Hordeum vulgare
agttgaactccgcgtcgatgaccgg

SEQ ID NO: 24 - DNA, 25 bp, Hordeum vulgare
ggagtccgggcggccatagagctgg

SEQ ID NO: 25 - DNA, 25 bp, Hordeum vulgare
cggcttccagcggtaccggaggtag

SEQ ID NO: 26 - DNA, 25 bp, Hordeum vulgare
gtcaaacctgggtagctcgcagctg

SEQ ID NO: 27 - DNA, 25 bp, Hordeum vulgare
cttcatccgcgtcaaaaagtcggcg

SEQ ID NO: 28 - DNA, 25 bp, Hordeum vulgare
tcccccacgaacatcaccgtcttcc

SEQ ID NO: 29 - DNA, 25 bp, Hordeum vulgare
cgactcccactggttacggcccagc

SEQ ID NO: 30 - DNA, 25 bp, Hordeum vulgare
gcgcggcggcgtgcagcaggcagac

SEQ ID NO: 31 - DNA, 25 bp, Hordeum vulgare
ggtcggcggagacgagctgcgacgg

SEQ ID NO: 32 - DNA, 25 bp, Hordeum vulgare
tcccctgcaccacgtcgatgtccac

SEQ ID NO: 33 - DNA, 25 bp, Hordeum vulgare
gaggtccacccagttggcccatgtg

SEQ ID NO: 34 - DNA, 25 bp, Hordeum vulgare
ttattgggcccagccaacgggaccg

SEQ ID NO: 35 - DNA, 25 bp, Hordeum vulgare
gctcctggcccatcggctgcgacgt

SEQ ID NO: 36 - DNA, 25 bp, Hordeum vulgare
ccctgcagcaccgttttcgtcacct

SEQ ID NO: 37 - DNA, 25 bp, Hordeum vulgare
gagcaaacggaccgggcttttcatc

SEQ ID NO: 38 - DNA, 25 bp, Hordeum vulgare
cgcagcgccgacagcgccgtgatgt

SEQ ID NO: 39 - DNA, 24 bp, Hordeum vulgare
ggggtgctggagaggcccaggtgg

SEQ ID NO: 40 - DNA, 25 bp, Artificial sequence
  synthetic
atgggggctcccgttaatccgaaga

SEQ ID NO: 41 - protein, 395 aa, Lactuca sativa
MAFQSKLISISTSFLILISTIFFHFTLLSSSYMLVHHINNNNEHNSHHRRPILQANQSQCSLFMGSWVHDDSYPIYQSSSCPVIDPDFNCQMYGRPDSDYLKYRWKPANCELPRFNGLEFLEKMKGKTVMFVGDSLGRNQWESLICLTWSSVPRVATQVIRAYPLSTFKFLDYGVTLSYYKAQYLVDIDSVGGRRVLKLQDISENAQAWRDVDVFVFNTGHWWSHTGATQGWDMMESGGSLYQDMDRMVAMETAVRTWARWVDSNIDTTKTRVFFQSFSPTHYDPSEWNGGSGSTTTAKSCYGETAPVSASAFLGVYPNEMKVVDAVLREMQTPVYMLDITTLSAMRKDAHPSIYSGDTSTGQKADPNGTPDCTHWCLPGLPDTWNQLFYTALFF

SEQ ID NO: 42 - protein, 399 aa, Solanum lycopersicum
MNLFSSLKFSSYSSSTKSFSFRFLLCLFLLNFFYVLTFASLRNHQYQQNHKKPIMILANLTSCSLFMGSWVYDETYPLYQSSSCPIIDSQFNCQMYGRPDTDYLKYRWKPTNCEIPRFNGLDFLLRMRGKTIMYVGDSLGRNQWQSLICMISAAVPRGAQTQFITGDPLSTYKFLDYGVSVSFYRTPYLVDIDSVQGKRILKLDDITKNSIAWRGVDVLSFNTGHWWTHKGALQGWDYMDLGGKLYQDMDRLVALERGLRTWARWIDANIDKSRTKLFFQGISPTHYNPSDWTTGTSTSSSTRTCYGETVPMTGTAYPGTFPDQMGVLKEVIGDMNNPPFILDITFLSAMRKDAHPSIYSGALTSEQKANPDHSADCSHWCLAGLPDTWNQLFYTALFF

SEQ ID NO: 43 - protein, 390 aa, Cucumis sativus
MGFLGFWPCLAALFFISLHTHLASSALILSLKHHYRNSDHHNRRPMLQANQTGCALFAGTWVRDDSYPLYQASNCPFIDPEFNCQAYGRPDSNYLKYRWQPLDCELPRFDGAEFLMRMRGRTVMFVGDSLGRNQWESLICLIVSSSPQTPTQMTRGEPLSTFRFLEYELTVSYYKAPYLVDIEIENGKRVLKLEEISMNGNAWVGADVISFNTGHWWSHTGSLQGWDYMESGGSYYQDMDRLGAMEKALRTWADWVEKNIDVSRTRVFFQAISPTHYNPSEWNTGTASMMTSTKNCYGETAPMGGTTYPGGYPIQMRVVDEVIREMRKPVYLLDITMLSELRKDGHPSIYSGDLNPQQRANPDRSADCSHWCLPGLPDTWNQLFYTALFF

SEQ ID NO: 44 - protein, 378 aa, Hordeum vulgare
MLLLCKSSVLAVLLLHAVSLSASALAVGLARRHRRDVLPGPKGCDVFSGSWVRDVDGSAAEGYTGYKCPVIDAEFNCQLYGRPDSDYLRYRWKPASCELPRFDGADFLTRMKGKTVMFVGDSLGRNQWESLVCLLHAAAPQSPSQLVSADPLYTYRFMEYELVVSFYRAPYLVDIDVVQGKRVLMLDYIAENAQAWRGADVLSFNSGHWWTHTGALQGWDYMGEGGRYSEDMDRMVAFQRGMTTWANWVDLNVDPAKTRVFFQSMSPTHYSSKEWPNPVSKNCYGETVPLAGPNNTSQPMGQEQVTKTVLQGMKSPVRLLDITALSALRKDAHPSVYSGDFSPAQRGNPAGSADCSHWCLPGLPDTWNQLFYTLLFYQ

SEQ ID NO: 45 - protein, 377 aa, Triticum aestivum
MLLLCKSSVLAVLLLHALSLPASALAVGLGRRHRRDVLPGPKGCDVFSGSWVRDDGSAEAAYTGYKCPVIDAEFNCQLYGRPDSDYLRYRWKPASCELPRFDGADFLTRMKGKTVMFVGDSLGRNQWESLVCLLHAAAPQSPSQLVSADPLYTYRFMEYELVVSFYRAPYLVDIDVVQGKRVLMLDDIAENAQAWRGADVLSFNSGHWWTHTGALQGWDYMGEGGRYSEDMDRMVAFQRGMTTWANWVDLNVDPAKTRVFFQSMSPTHYSSKEWPNPVSKNCYGETVPLAGLNSTAQPMGQEQVTKTVLQGMKSPVRLLDITALSALRKDAHPSVYSGDFSPAQRGNPAGSADCSHWCLPGLPDTWNQLFYTLLFYQ

SEQ ID NO: 46 - protein, 392 aa, Cucumis melo
MGFLGFWPYLAALFFISLHTHLASSALILSLKHHYRNSDHHNRRPMLQANQTSCALFAGTWVRDNSYPLYQASNCPFIDPEFNCQAYGRPDSNYLQYRWQPLDCELPRFDGAEFLMRMRGRTVMFVGDSLGRNQWESLICLIFSSSPQTPTQMTRGEPLSTFRFLEYELTVSYYKAPYLVDIEIENGKRVLKLEEISMNGNAWVGADVISFNTGHWWSHTGSLQGWDYMESGGSYYQDMDRLGAMEKALRTWADWVEKNIDVSRTRVFFQAISPTHYNPSEWNTGTGTASMMTSTKNCYGETAPMGGTTYPGAYPIQMRVVDEVIREMRKPVYLLDITMLSELRKDGHPSIYSGDLNPQQRANPDRSADCSHWCLPGLPDTWNQLFYTALFF

SEQ ID NO: 47 - protein, 391 aa, Zea mays
MLPSRRKSAVLGVVALSSSVLILLHAFSLPAASALSVGLARRHRHDATPGLAAGGCDVFSGSWVLDGGSSASAAYTGYNCPLIDAEFNCQLYGRPDSDYLRYLWKPAGCELPRFDGADFLTRMKGKTVMFVGDSLGRNQWESLVCLLHAAAPQSPAQLVSADPLYTYKFLEYQVTVSFYRAPYLVDIDVVQGKRVLMLDDISENAESWRDADVLSFNSGHWWTHTGSMQGWDYMGESGRYYEDMDRTVAFQRGLTTWASWVDLNLDQAKTRVFFQSMSPTHYSSKEWPNPVSKNCYGETAPATGLNSTGQASGQDQVIQAVLRGMKSPVRLLDITALSAMRKDAHPSAYSGDFSPAQRANPGAGSVDCSHWCLPGLPDTWNQLFYTLLFYK

SEQ ID NO: 48 - protein, 388 aa, Glycine max
MALLHSLFALLFLLLSLQLHTVSCALLLSLRHHHNTLHQQRPMIHANQTNCALFVGTWVQDDSYPIYQSSNCPIIDPQFNCKMFGRPDSDYLRYRWRPLNCDLPRFNGVEFLLQMKGKTVMFVGDSLGRNQWQSLICMIYAAVPQTQTQLVRGEPLSTFRFLDYGVTISFYRAPYLVEIDVVQGKRILRLEEVDGNGDVWRSVDVLSFNTGHWWDHQGSLQGWDYMELGGKYYQDMDRLAALERGMKTWANWVDSNVDRSRTKVFFLGISPSHTNPNEWNSGVTAGLTTKNCYGETTPITSTGTAYPGVYPEQMRVVDMVIRGMSNPAYLLDITMLSAFRKDAHPSIYSGDLNPQQRANPTYSADCSHWCLPGLPDTWNELFYTTLFY

SEQ ID NO: 49 - protein, 393 aa, Oryza sativa
MPLFCKHGAGFLAVAVSTSIVFLLLLHAFSLPASALSFGLARRHRKDAAVSCDVFSGSWVRDDGGGGAAAYTGYNCPVIDAEFNCQLYGRPDSDYLRYRWKPAGCELPRFDGADFLTRMKGKTVMFVGDSLGRNQWESLICLLHAAAPQSPAQLVSVDPLYTYKFLEYGLVVSFYRAPYLVDIGVAQGKKVLWLDDISGNGEAWRGADVLSFNSGHWWTHTGTLQGWDYMGADGRYYEDMDRMVAFQRGMATWANWVDANVDPAKTRVFYQSMSPTHYSSKEWANPVSKNCYGETAPAAAAAAGLNATTAQATSGQDQVIQATLRSMKSPVRLLDISALSALRKDAHPSVYSGDLSPAQRANPGGGSADCSHWCLPGLPDTWNQLFYALLFYQ

SEQ ID NO: 50 - DNA, 25 bp, Artificial sequence
  synthetic
agcgccggtagcgagcatacgtatg

SEQ ID NO: 51 - DNA, 25 bp, Artificial sequence
  synthetic
acgactctgcttattatactcggtc

SEQ ID NO: 52 - DNA, 25 bp, Artificial sequence
  synthetic
gacatattaggggcgacgtctccaa

SEQ ID NO: 53 - RNA, 150 nt, Cucumis sativus
augggguuuuugggauuuuggccuuguuuggcagcauuguuuuucauuucucuucauacccauuuagcuuccucagcuuugauucucaguuugaagcaccauuaccggaacagcgaccaccauaaucgccgcccaaugcuucaagcuaac

SEQ ID NO: 54 - RNA, 150 nt, Cucumis sativus
uauggggaaacggcaccaaugggggggacgacguacccgggaggguacccuauucaaaugaggguuguggaugaagugauaagggagaugaggaagccaguauacuuauuggacauaacaauguuaucugagcuaagaaaagauggacac

SEQ ID NO: 55 - RNA, 150 nt, Cucumis sativus
aaaccggcugugcucuguucgccggaacuuggguucgagaugauucauauccacuuuaccaagcuucuaacuguccauuuaucgauccugaguucaacugccaagcuuacggcagacccgauucaaauuaccucaaguaccguuggcagc

SEQ ID NO: 56 - RNA, 113 nt, Cucumis sativus
aaggcuuuaagaacaugggcugauuggguugagaagaacauugaugucucuagaacaaggguuuucuuccaagcuaucucccccacacauuacaauccaucugaauggaacac

SEQ ID NO: 57 - DNA, 25 bp, Glycine max
ctacaattaaatttaatgacatatt

SEQ ID NO: 58 - DNA, 25 bp, Glycine max
gacggaaggattagtgaattatgtg

SEQ ID NO: 59 - DNA, 25 bp, Glycine max
gaaattagagatgacaatcacaaaa

SEQ ID NO: 60 - DNA, 25 bp, Glycine max
taatgtgttgaacatgtggttagca

SEQ ID NO: 61 - DNA, 25 bp, Glycine max
gaaaaacatgactagtcttgggaca

SEQ ID NO: 62 - DNA, 25 bp, Glycine max
gagagaagagggtatgtgtaatgag

SEQ ID NO: 63 - DNA, 25 bp, Glycine max
tacacccctaaatttgatttgttat

SEQ ID NO: 64 - DNA, 25 bp, Glycine max
tgactgagacaaaaatgcacaccta

SEQ ID NO: 65 - DNA, 25 bp, Glycine max
tttattcttaccttcaggatctcaa

SEQ ID NO: 66 - DNA, 25 bp, Glycine max
aacaactctcctcctatattaaaat

SEQ ID NO: 67 - DNA, 25 bp, Glycine max
atccattttctcatcaacaactaaa

SEQ ID NO: 68 - DNA, 25 bp, Glycine max
cttttttcttatgagagggacgaac

SEQ ID NO: 69 - DNA, 25 bp, Glycine max
aaaggcatgtacaaaagaagtctaa

SEQ ID NO: 70 - DNA, 25 bp, Glycine max
ttaaacataatatatttacatcagt

SEQ ID NO: 71 - DNA, 25 bp, Glycine max
attgtaaacatcttcattctatctt

SEQ ID NO: 72 - DNA, 25 bp, Glycine max
cttctttacatgtacacaacaaaac

SEQ ID NO: 73 - DNA, 25 bp, Glycine max
tgtgcactttttgtgggaacttggg

SEQ ID NO: 74 - DNA, 25 bp, Glycine max
ccaagatgactcttaccctatctat

SEQ ID NO: 75 - DNA, 25 bp, Glycine max
tcctctaactgccccatcatagatc

SEQ ID NO: 76 - DNA, 25 bp, Glycine max
cagttcaactgcaagatgtttggcc

SEQ ID NO: 77 - DNA, 25 bp, Glycine max
cctgattctgattacctcagataca

SEQ ID NO: 78 - DNA, 25 bp, Glycine max
atggagacccctcaactgtgacctc

SEQ ID NO: 79 - DNA, 25 bp, Glycine max
ctaggttcaatggggtggagtttct

SEQ ID NO: 80 - DNA, 25 bp, Glycine max
gcaaatgaagggcaaaactgtgatg

SEQ ID NO: 81 - DNA, 25 bp, Glycine max
ttgtgggtgactcactagggcgcaa

SEQ ID NO: 82 - DNA, 25 bp, Glycine max
caatggcaatcactgatttgcatga

SEQ ID NO: 83 - DNA, 25 bp, Glycine max
tatgctgcagttcctcagacacaaa

SEQ ID NO: 84 - DNA, 25 bp, Glycine max
caattggtcagaggggaaccactct

SEQ ID NO: 85 - DNA, 25 bp, Glycine max
ggtgggatcatcaaggctctcttca

SEQ ID NO: 86 - DNA, 25 bp, Glycine max
ggtgggattatatggaattaggagg

SEQ ID NO: 87 - DNA, 25 bp, Glycine max
tacaaagaactgctatggtgaaact

SEQ ID NO: 88 - DNA, 25 bp, Glycine max
ctccaattactagcactggcacagc

SEQ ID NO: 89 - DNA, 25 bp, Glycine max
taccctggtgtgtaccctgaacaaa

SEQ ID NO: 90 - DNA, 25 bp, Glycine max
gagggttgtggacatggtaattaga

SEQ ID NO: 91 - DNA, 25 bp, Glycine max
ggaatgagtaaccctgcttatcttc

SEQ ID NO: 92 - DNA, 25 bp, Glycine max
tgacatcacaatgctgtcagcattt

SEQ ID NO: 93 - DNA, 25 bp, Glycine max
aatatgtcattaaatttaattgtag

SEQ ID NO: 94 - DNA, 25 bp, Glycine max
cacataattcactaatccttccgtc

SEQ ID NO: 95 - DNA, 25 bp, Glycine max
ttttgtgattgtcatctctaatttc

SEQ ID NO: 96 - DNA, 25 bp, Glycine max
tgctaaccacatgttcaacacatta

SEQ ID NO: 97 - DNA, 25 bp, Glycine max
tgtcccaagactagtcatgtttttc

SEQ ID NO: 98 - DNA, 25 bp, Glycine max
ctcattacacataccctcttctctc

SEQ ID NO: 99 - DNA, 25 bp, Glycine max
ataacaaatcaaatttaggggtgta

SEQ ID NO: 100 - DNA, 25 bp, Glycine max
taggtgtgcatttttgtctcagtca

SEQ ID NO: 101 - DNA, 25 bp, Glycine max
ttgagatcctgaaggtaagaataaa

SEQ ID NO: 102 - DNA, 25 bp, Glycine max
attttaatataggaggagagttgtt

SEQ ID NO: 103 - DNA, 25 bp, Glycine max
tttagttgttgatgagaaaatggat

SEQ ID NO: 104 - DNA, 25 bp, Glycine max
gttcgtccctctcataagaaaaaag

SEQ ID NO: 105 - DNA, 25 bp, Glycine max
ttagacttcttttgtacatgccttt

SEQ ID NO: 106 - DNA, 25 bp, Glycine max
actgatgtaaatatattatgtttaa

SEQ ID NO: 107 - DNA, 25 bp, Glycine max
aagatagaatgaagatgtttacaat

SEQ ID NO: 108 - DNA, 25 bp, Glycine max
gttttgttgtgtacatgtaaagaag

SEQ ID NO: 109 - DNA, 25 bp, Glycine max
cccaagttcccacaaaaagtgcaca

SEQ ID NO: 110 - DNA, 25 bp, Glycine max
atagatagggtaagagtcatcttgg

SEQ ID NO: 111 - DNA, 25 bp, Glycine max
gatctatgatggggcagttagagga

SEQ ID NO: 112 - DNA, 25 bp, Glycine max
ggccaaacatcttgcagttgaactg

SEQ ID NO: 113 - DNA, 25 bp, Glycine max
tgtatctgaggtaatcagaatcagg

SEQ ID NO: 114 - DNA, 25 bp, Glycine max
gaggtcacagttgaggggtctccat

SEQ ID NO: 115 - DNA, 25 bp, Glycine max
agaaactccaccccattgaacctag

SEQ ID NO: 116 - DNA, 25 bp, Glycine max
catcacagttttgcccttcatttgc

SEQ ID NO: 117 - DNA, 25 bp, Glycine max
ttgcgccctagtgagtcacccacaa

SEQ ID NO: 118 - DNA, 25 bp, Glycine max
tcatgcaaatcagtgattgccattg

SEQ ID NO: 119 - DNA, 25 bp, Glycine max
tttgtgtctgaggaactgcagcata

SEQ ID NO: 120 - DNA, 25 bp, Glycine max
agagtggttcccctctgaccaattg

SEQ ID NO: 121 - DNA, 25 bp, Glycine max
tgaagagagccttgatgatcccacc

SEQ ID NO: 122 - DNA, 25 bp, Glycine max
cctcctaattccatataatcccacc

SEQ ID NO: 123 - DNA, 25 bp, Glycine max
agtttcaccatagcagttctttgta

SEQ ID NO: 124 - DNA, 25 bp, Glycine max
gctgtgccagtgctagtaattggag

SEQ ID NO: 125 - DNA, 25 bp, Glycine max
tttgttcagggtacacaccagggta

SEQ ID NO: 126 - DNA, 25 bp, Glycine max
tctaattaccatgtccacaaccctc

SEQ ID NO: 127 - DNA, 25 bp, Glycine max
gaagataagcagggttactcattcc

SEQ ID NO: 128 - DNA, 25 bp, Glycine max
aaatgctgacagcattgtgatgtca

(19) United States
(12) Patent Application Publication
(10) Pub. No.: US 2019/0008156 A1
(43) Pub. Date: Jan. 10, 2019

(54) Methods and Compositions for Plant Pest Control

(71) Applicant: Monsanto Technology LLC, St. Louis, MO (US)

(72) Inventors: Michael J. Crawford, St. Louis, MO (US); Xiangqian Li, St. Louis, MO (US); Barry J. Shortt, St. Louis, MO (US); Deryck Jeremy Williams, St. Louis, MO (US)

(21) Appl. No.: 16/141,618

(22) Filed: Sep. 25, 2018

Related U.S. Application Data
(62) Division of application No. 14/760,738, filed on Jul. 14, 2015.
(60) Provisional application No. 61/752,703, filed on Jan. 15, 2013.

(51) Int. Cl. A01N 57/16; C12N 15/82; A01N 63/00; C12N 15/10; C07K 14/415; A01N 37/46

(52) U.S. Cl. CPC A01N 57/16; C12N 15/8285; C12N 15/8282; A01N 63/00; Y02A 40/164; C12N 15/8218; C07K 14/415; A01N 37/46; C12N 15/1093

(57) ABSTRACT

Provided are methods and compositions to improve fungal disease resistance and/or nematode resistance in various crop plants. Also provided are combinations of compositions and methods to improve fungal disease resistance and/or nematode resistance in various crop plants. Powdery mildews are fungal diseases that affect a wide range of plants including cereals, grasses, vegetables, ornamentals, weeds, shrubs, fruit trees, broad-leaved shade and forest trees, that is caused by different species of fungi in the order Erysiphales.

### CROSS-REFERENCE TO RELATED APPLICATIONS

This international patent application claims the benefit of U.S. Provisional patent Application No. 61/752,703, filed Jan. 15, 2013, which is incorporated herein by reference in its entirety.

### INCORPORATION OF SEQUENCE LISTING

A sequence listing is provided herewith as a part of this International Patent application via the USPTO's EFS system in file named “40_71_59225_SEQ_LISTING” which is 70,527 bytes in size (measured in MS-Windows®), contains 128 sequences, was created on Jan. 14, 2014, and is incorporated herein by reference in its entirety.

### BACKGROUND

Powdery mildews are fungal diseases that affect a wide range of plants including cereals, grasses, vegetables, ornamentals, weeds, shrubs, fruit trees, broad-leaved shade and forest trees, that is caused by different species of fungi in the order Erysiphales. The disease is characterized by spots or patches of white to grayish, talcum-powder-like growth that produce tiny, pinhead-sized, spherical fruiting structures (the cleistothecia or overwintering bodies of the fungus), that are first white, later yellow-brown and finally black. The fungi that cause powdery mildews are host specific and cannot survive without the proper host plant. They produce mycelium (fungal threads) that grow only on the surface of the plant and feed by sending haustoria, or root-like structures, into the epidermal cells of the plant. The fungi overwinter on plant debris as cleistothecia or mycelia. In the spring, the cleistothecia produce spores that are moved to susceptible hosts by rain, wind or insects.

Powdery mildew disease is particularly prevalent in temperate and humid climates, where they frequently cause significant yield losses and quality reductions in various agricultural settings including greenhouse and field farming. This affects key cereals (e.g. barley and wheat), horticultural crops (e.g. grapevine, pea and tomato) and economically important ornamentals (e.g. roses). Limited access to natural sources of resistance to powdery mildews, rapid changes in pathogen virulence and the time consuming introgression of suitable resistance genes into elite varieties has led to the widespread use of fungicides to control the disease. This has not surprisingly led to the evolution and spread of fungicide resistance, which is especially dramatic amongst the most economically important powdery mildews.

Downy mildew diseases are caused by oomycete microbes from the family Peronosporaceae that are parasites of plants. Peronosporaceae are obligate biotrophic plant pathogens and parasitize their host plants as an intercellular mycelium using haustoria to penetrate the host cells. The downy mildews reproduce asexually by forming sporangia on distinctive white sporangiophores usually formed on the lower surface of infected leaves. These constitute the “downy mildew” and the initial symptoms appear on leaves as light green to yellow spots. The sporangia are wind-dispersed to the surface of other leaves. Depending on the genus, the sporangia may germinate by forming zoospores or by germ-tube. In the latter case, the sporangia behave like fungal conidia and are often referred to as such. Sexual reproduction is via oospores.

Most Peronosporaceae are pathogens of herbaceous dicots. Some downy mildew genera have relatively restricted host ranges, e.g. Basidiophora, Paraperonospora, Protobremia and Bremia on Asteraceae; Perofascia and Hyaloperonospora almost exclusively on Brassicaceae; Viennotia, Graminivora, Poakatesthia, Sclerospora and Peronosclerospora on Poaceae, Plasmoverna on Ranunculaceae. However, the largest genera, Peronospora and Plasmopara, have very wide host ranges.

In commercial agriculture, downy mildews are a particular problem for growers of crucifers, grapes and vegetables that grow on vines. Peronosporaceae of economic importance include Plasmopara viticola which infect grapevines, Peronospora tabacina which causes blue mold on tobacco, Bremia lactucae, a parasite on lettuce, and Plasmopara halstedii on sunflower.

Rusts (Pucciniales, formerly Uredinales) are obligate biotrophic parasites of vascular plants. Rusts affect a variety of plants; leaves, stems, fruits and seeds and is most commonly seen as coloured powder, composed of tiny aeciospores which land on vegetation producing pustules, or uredia, that form on the lower surfaces. During late spring or early summer, yellow orange or brown, hairlike or ligulate structures called telia grow on the leaves or emerge from bark of woody hosts. These telia produce teliospores which will germinate into aerial basidiospores, spreading and causing further infection.

### SUMMARY

The present embodiments provide for compositions comprising polynucleotide molecules and methods for treating a plant to alter or regulate gene or gene transcript expression in the plant, for example, by providing RNA or DNA for inhibition of expression. Various aspects provide compositions comprising polynucleotide molecules and related methods for topically applying such compositions to plants to regulate endogenous PMR5 genes in a plant cell. The polynucleotides, compositions, and methods disclosed herein are useful in decreasing levels of PMR5 transcript and improving fungal disease and/or nematode resistance of a plant.

In one aspect, the polynucleotide molecules are provided in compositions that can permeate or be absorbed into living plant tissue to initiate localized, partially systemic, or systemic gene inhibition or regulation. In certain embodiments, the polynucleotide molecules ultimately provide to a plant, or allow the in planta production of, RNA that is capable of hybridizing under physiological conditions in a plant cell to RNA transcribed from a target endogenous gene or target transgene in the plant cell, thereby effecting regulation of the endogenous PMR5 target gene. In certain embodiments, regulation of the PMR5 target genes, such as by silencing or suppression of the target gene, leads to the upregulation of another gene that is itself affected or regulated by decreasing the PMR5 target gene's expression.

In some embodiments, the topical application of a composition comprising an exogenous polynucleotide and a transfer agent to a plant or plant part according to the methods described herein does not necessarily result in nor require the exogenous polynucleotide's integration into a chromosome of the plant. In some embodiments, the topical application of a composition comprising an exogenous polynucleotide and a transfer agent to a plant or plant part according to the methods described herein does not necessarily result in nor require transcription of the exogenous polynucleotide from DNA integrated into a chromosome of the plant. In certain embodiments, topical application of a composition comprising an exogenous polynucleotide and a transfer agent to a plant according to the methods described herein also does not necessarily require that the exogenous polynucleotide be physically bound to a particle, such as in biolistic mediated introduction of polynucleotides associated with a gold or tungsten particles into internal portions of a plant, plant part, or plant cell. An exogenous polynucleotide used in certain methods and compositions provided herein can optionally be associated with an operably linked promoter sequence in certain embodiments of the methods provided herein. However, in other embodiments, an exogenous polynucleotide used in certain methods and compositions provided herein is not associated with an operably linked promoter sequence. Also, in certain embodiments, an exogenous polynucleotide used in certain methods and compositions provided herein is not operably linked to a viral vector.

In certain embodiments, methods for improving fungal disease resistance and/or nematode resistance in a plant comprising topically applying compositions comprising a polynucleotide and a transfer agent that suppress the target PMR5 gene are provided. In certain embodiments, methods for selectively suppressing the target PMR5 gene by topically applying the polynucleotide composition to a plant surface at one or more selected seed, vegetative, or reproductive stage(s) of plant growth are provided. Such methods can provide for gene suppression in a plant or plant part on an as needed or as desired basis. In certain embodiments, methods for selectively suppressing the target PMR5 gene by topically applying the polynucleotide composition to a plant surface at one or more pre-determined seed, vegetative, or reproductive stage(s) of plant growth are provided. Such methods can provide for PMR5 gene suppression in a plant or plant part that obviates any undesired or unnecessary effects of suppressing the genes expression at certain seed, vegetative, or reproductive stage(s) of plant development.

In certain embodiments, methods for selectively improving fungal disease resistance and/or nematode resistance in a plant by topically applying the polynucleotide composition to the plant surface at one or more selected seed, vegetative, or reproductive stage(s) are provided. Such methods can provide for improved fungal disease resistance and/or nematode disease resistance in a plant or plant part on an as needed or as desired basis. In certain embodiments, methods for selectively improving fungal disease and/or nematode resistance in a plant by topically applying the polynucleotide composition to the plant surface at one or more predetermined seed, vegetative, or reproductive stage(s) are provided. Such methods can provide for improving fungal disease and/or nematode resistance in a plant or plant part that obviates any undesired or unnecessary effects of suppressing PMR5 gene expression at certain seed, vegetative, or reproductive stage(s) of plant development.

Polynucleotides that can be used to suppress a PMR5 include, but are not limited to, any of: i) polynucleotides comprising at least 18 contiguous nucleotides that are essentially identical or essentially complementary to a gene or a transcript of the gene(s) of Table 2 (SEQ ID NO: 1, 2, 3, 4, 5, 6, 7, 8, 9, 10, or 11) or encoding a protein of Table 3 (SEQ ID NO: 41-48, or 49); ii) polynucleotides comprising at least 18 contiguous nucleotides that are essentially identical or essentially complementary to a gene encoding a PMR5 or PMR5-like protein of Table 3 comprising a polynucleotide of SEQ ID NO:41-48, or 49; or, polynucleotides comprising at least 18 contiguous nucleotides that are essentially identical or essentially complementary to a polynucleotides of SEQ ID NO:12-19, 21-37, 53-127 or 128. Methods and compositions that provide for the topical application of certain polynucleotides in the presence of transfer agents can be used to suppress PMR5 gene expression in an optimal manner. In certain embodiments, the compositions provided herein can be applied on an “as needed” basis upon scouting for the occurrence of fungal disease or nematodes. In certain embodiments, the compositions can be applied in a manner that obviates any deleterious effects on yield or other characteristics that can be associated with suppression of PMR5 gene expression in a crop plant. The applied polynucleotides are complementary to the PMR5 target host gene in plants and their topical application leads to suppression of the PMR5 gene's activity.

Provided herein are compositions and methods for controlling plant fungal diseases. Plant fungal diseases that can be controlled with the methods and compositions provided herein include, but are not limited to, obligate biotrophic powdery mildew, downy mildew and rust fungal infestations in plants. In certain embodiments, methods and compositions for reducing expression of one or more host plant PMR5 polynucleotide and/or protein molecules in one or more cells or tissues of the plant such that the plant is rendered less susceptible to fungal infections from the order Erysiphales, the family Peronosporaceae or the order Pucciniales, are provided. In certain embodiments, nucleotide and amino acid sequences of plant PMR5 genes which can be downregulated by methods and compositions provided herein to increase plant resistance to powdery mildew, downy mildew or rust infection are disclosed.

Also provided herein are methods and compositions that provide for reductions in expression of PMR5 target polynucleotide and protein molecules in at least the cells of a plant root and for improved resistance to nematodes. Nematodes that can be controlled by the methods and compositions provided herein include, but are not limited to, root knot nematodes (such as Meloidogyne sp.), cyst nematodes (such as Globodera sp. and Heterodera sp.), lesion nematodes (such as Pratylenchus sp.), and the like. In certain embodiments, PMR5 expression is reduced in plant root cells from which nematodes feed by providing topically to plant leaves, shoots, roots and/or seeds compositions comprising polynucleotides that comprise at least 18 contiguous nucleotides that are essentially identical or essentially complementary to a PMR5 gene or to a transcript of the PMR5 gene; and a transfer agent.

Also provided are methods and compositions where topically induced reductions in PMR5 transcript or protein levels are used to achieve powdery mildew, downy mildew or rust control while minimizing deleterious pleotropic effects in the host plant. Such methods and compositions provide for optimized levels of PMR5 gene inhibition and/or optimized timing of PMR5 gene inhibition.

Certain embodiments are directed to methods for producing a plant exhibiting an improvement in fungal disease resistance and/or nematode resistance comprising topically applying to a plant surface a composition that comprises:

a. at least one polynucleotide that comprises at least 18 contiguous nucleotides that are essentially identical or essentially complementary to a PMR5 gene or to a transcript of the gene; and

b. a transfer agent, wherein the plant exhibits an improvement in fungal disease resistance and/or nematode resistance that results from suppression of the PMR5 gene. In certain embodiments, the polynucleotide molecule comprises sense ssDNA, sense ssRNA, dsRNA, dsDNA, a double stranded DNA/RNA hybrid, anti-sense ssDNA, or anti-sense ssRNA. In certain embodiments, the polynucleotide is selected from the group consisting of SEQ ID NO: 12-19, 21-37, 53-127, or 128, or wherein the polynucleotide comprises at least 18 contiguous nucleotides that are essentially identical or essentially complementary to SEQ ID NO: 1, 2, 3, 4, 5, 6, 7, 8, 9, 10, or 11. In certain embodiments: (a) the plant is a soybean plant, the gene or the transcript is a soybean PMR5 gene or transcript, and the polynucleotide molecule is selected from the group consisting of SEQ ID NO:12-19, 57-127 and SEQ ID NO:128, or the polynucleotide comprises at least 18 contiguous nucleotides that are essentially identical or essentially complementary to SEQ ID NO:8 or 11; (b) the plant is a barley plant, the gene or the transcript is a barley PMR5 gene or transcript, and the polynucleotide molecule is selected from the group consisting of SEQ ID NO:21-37, and SEQ ID NO:38, or the polynucleotide comprises at least 18 contiguous nucleotides that are essentially identical or essentially complementary to SEQ ID NO:4; (c) the plant is a cucumber plant, the gene or the transcript is a cucumber PMR5 gene or transcript, and the polynucleotide comprises at least 18 contiguous nucleotides that are essentially identical or essentially complementary to SEQ ID NO:3, or 6, 53, 54, 55, or 56; (d) the plant is a lettuce plant, the gene or the transcript is a lettuce PMR5 gene or transcript, and the polynucleotide comprises at least 18 contiguous nucleotides that are essentially identical or essentially complementary to SEQ ID NO:1; (e) the plant is a corn plant, the gene or the transcript is a corn PMR5 gene or transcript, and the polynucleotide comprises at least 18 contiguous nucleotides that are essentially identical or essentially complementary to SEQ ID NO:7; (f) the plant is a tomato plant, the gene or the transcript is a tomato PMR5 gene or transcript, and the polynucleotide comprises at least 18 contiguous nucleotides that are essentially identical or essentially complementary to SEQ ID NO:2 or 10; (g) the plant is a wheat plant, the gene or the transcript is a wheat PMR5 gene or transcript, and the polynucleotide comprises at least 18 contiguous nucleotides that are essentially identical or essentially complementary to SEQ ID NO:5; or, (h) the plant is a rice plant, the gene or the transcript is a rice PMR5 gene or transcript, and the polynucleotide comprises at least 18 contiguous nucleotides that are essentially identical or essentially complementary to SEQ ID NO:9. In certain embodiments, the composition comprises any combination of two or more polynucleotide molecules. In certain embodiments, the polynucleotide is at least 18 to about 24, about 25 to about 50, about 51 to about 100, about 101 to about 300, about 301 to about 500, or at least about 500 or more residues in length. In certain embodiments, the composition further comprises a non-polynucleotide herbicidal molecule, a polynucleotide herbicidal molecule, a polynucleotide that suppresses an herbicide target gene, an insecticide, a fungicide, a nematocide, or a combination thereof. In certain embodiments, the composition further comprises a non-polynucleotide herbicidal molecule and the plant is resistant to the herbicidal molecule. In certain embodiments, the transfer agent comprises an organosilicone preparation. In certain embodiments, the polynucleotide is not operably linked to a viral vector. In certain embodiments, the polynucleotide is not integrated into the plant chromosome. Further embodiments are directed to: a plant made according to any of the above-described methods; progeny of plants that exhibit the improvements in fungal disease resistance and/or nematode resistance; seed of the plants, wherein seed from the plants exhibits the improvement in fungal disease resistance and/or nematode resistance; and a processed product of the plants, the progeny plants, or the seeds, wherein the processed products exhibit the improvement in fungal disease resistance and/or nematode resistance. In certain embodiments, the processed product of the plant or plant part exhibits an improved attribute relative to a processed product of an untreated control plant and the improved attribute results from the improved fungal disease resistance and/or nematode resistance. An improved attribute of a processed product can include, but is not limited to, decreased mycotoxin content, improved nutritional content, improved storage characteristics, improved flavor, improved consistency, and the like when compared to a processed product obtained from an untreated plant or plant part.

An additional embodiment is directed to a composition comprising a polynucleotide molecule that comprises at least 18 contiguous nucleotides that are essentially identical or essentially complementary to a PMR5 gene or transcript of the gene, wherein the polynucleotide is not operably linked to a promoter; and, b) a transfer agent. In certain embodiments, the polynucleotide is selected from the group consisting of SEQ ID NO: 12-19, 21-38, 53-127, and 128, or the polynucleotide comprises at least 18 contiguous nucleotides that are essentially identical or essentially complementary to SEQ ID NO: 1, 2, 3, 4, 5, 6, 7, 8, 9, 10, or 11, or wherein the polynucleotide comprises at least 18 contiguous nucleotides that are essentially identical or essentially complementary to SEQ ID NO: 1, 2, 3, 4, 5, 6, 7, 8, 9, 10, or 11. In certain embodiments: (a) the plant is a soybean plant, the gene or the transcript is a soybean PMR5 gene or transcript, and the polynucleotide molecule is selected from the group consisting of SEQ ID NO:12-19, 57-127, and SEQ ID NO:128, or the polynucleotide comprises at least 18 contiguous nucleotides that are essentially identical or essentially complementary to SEQ ID NO:8 or 11; (b) the plant is a barley plant, the gene or the transcript is a barley PMR5 gene or transcript, and the polynucleotide molecule is selected from the group consisting of SEQ ID NO:21-37, and SEQ ID NO:38, or the polynucleotide comprises at least 18 contiguous nucleotides that are essentially identical or essentially complementary to SEQ ID NO:4; (c) the plant is a cucumber plant, the gene or the transcript is a cucumber PMR5 gene or transcript, and the polynucleotide comprises at least 18 contiguous nucleotides that are essentially identical or essentially complementary to SEQ ID NO:3, or 6, 53, 54, 55, or 56; (d) the plant is a lettuce plant, the gene or the transcript is a lettuce PMR5 gene or transcript, and the polynucleotide comprises at least 18 contiguous nucleotides that are essentially identical or essentially complementary to SEQ ID NO:1; (e) the plant is a corn plant, the gene or the transcript is a corn PMR5 gene or transcript, and the polynucleotide comprises at least 18 contiguous nucleotides that are essentially identical or essentially complementary to SEQ ID NO:7; (f) the plant is a tomato plant, the gene or the transcript is a tomato PMR5 gene or transcript, and the polynucleotide comprises at least 18 contiguous nucleotides that are essentially identical or essentially complementary to SEQ ID NO:2 or 10; (g) the plant is a wheat plant, the gene or the transcript is a wheat PMR5 gene or transcript, and the polynucleotide comprises at least 18 contiguous nucleotides that are essentially identical or essentially complementary to SEQ ID NO:5; or, (h) the plant is a rice plant, the gene or the transcript is a rice PMR5 gene or transcript, and the polynucleotide comprises at least 18 contiguous nucleotides that are essentially identical or essentially complementary to SEQ ID NO:9. In certain embodiments, the polynucleotide is at least 18 to about 24, about 25 to about 50, about 51 to about 100, about 101 to about 300, about 301 to about 500, or at least about 500 or more residues in length. In certain embodiments, the composition further comprises a non-polynucleotide herbicidal molecule, a polynucleotide herbicidal molecule, a polynucleotide that suppresses an herbicide target gene, an insecticide, a fungicide, a nematocide, or a combination thereof. In certain embodiments, the transfer agent is an organo silicone preparation. In certain embodiments, the polynucleotide is not physically bound to a biolistic particle.

Another embodiment is directed to a method of making a composition comprising the step of combining at least: (a) a polynucleotide molecule comprising at least 18 contiguous nucleotides that are essentially identical or essentially complementary to a PMR5 gene or transcript of a plant, wherein the polynucleotide is not operably linked to a promoter or a viral vector; and, (b) a transfer agent. In certain embodiments, the polynucleotide is obtained by in vivo biosynthesis, in vitro enzymatic synthesis, or chemical synthesis. In certain embodiments, the method further comprises combining with the polynucleotide and the transfer agent at least one of a non-polynucleotide herbicidal molecule, a polynucleotide herbicidal molecule, an insecticide, a fungicide, and/or a nematocide. In certain embodiments, the transfer agent is an organosilicone preparation.

Yet another embodiment is directed to a method of identifying a polynucleotide for improving fungal disease resistance and/or nematode resistance in a plant comprising; (a) selecting a population of polynucleotides that are essentially identical or essentially complementary to a PMR5 gene or transcript of a plant; (b) topically applying to a surface of at least one of the plants a composition comprising at least one polynucleotide from the population and an transfer agent to obtain a treated plant; and, (c) identifying a treated plant that exhibits suppression of the PMR5 gene or exhibits an improvement in fungal disease resistance or exhibits an improvement in nematode resistance, thereby identifying a polynucleotide that improves fungal disease resistance and/or nematode resistance in the plant. In certain embodiments, the selection of the population of polynucleotides that are essentially identical or essentially complementary to a PMR5 gene or transcript of a plant can be effected by identifying polynucleotides that can suppress a PMR5 gene via Virus Induced Gene Silencing (VIGS). Those polynucleotides that can suppress a PMR5 gene via VIGS are disassociated from the VIGS vector, topically applied to a surface of a plant in a composition comprising at least one of those polynucleotides and a treated plant that exhibits suppression of the PMR5 gene or exhibits an improvement in fungal disease resistance or nematode resistance is identified, thus identifying a polynucleotide that improves fungal disease resistance and/or nematode resistance in the plant. In certain embodiments, the polynucleotide is selected from the group consisting of SEQ ID NO: 12-19, 21-38, 53-127, and 128, or wherein the polynucleotide comprises at least 18 contiguous nucleotides that are essentially identical or essentially complementary to SEQ ID NO: 1, 2, 3, 4, 5, 6, 7, 8, 9, 10, or 11. In certain embodiments: (a) the plant is a soybean plant, the gene or the transcript is a soybean PMR5 gene or transcript, and the polynucleotide molecule is selected from the group consisting of SEQ ID NO:12-19, 57-127, and SEQ ID NO:128, or the polynucleotide comprises at least 18 contiguous nucleotides that are essentially identical or essentially complementary to SEQ ID NO:8 or 11; (b) the plant is a barley plant, the gene or the transcript is a barley PMR5 gene or transcript, and the polynucleotide molecule is selected from the group consisting of SEQ ID NO:21-37, and SEQ ID NO:38, or the polynucleotide comprises at least 18 contiguous nucleotides that are essentially identical or essentially complementary to SEQ ID NO:4; (c) the plant is a cucumber plant, the gene or the transcript is a cucumber PMR5 gene or transcript, and the polynucleotide comprises at least 18 contiguous nucleotides that are essentially identical or essentially complementary to SEQ ID NO:3, or 6, 53, 54, 55, or 56; (d) the plant is a lettuce plant, the gene or the transcript is a lettuce PMR5 gene or transcript, and the polynucleotide comprises at least 18 contiguous nucleotides that are essentially identical or essentially complementary to SEQ ID NO:1; (e) the plant is a corn plant, the gene or the transcript is a corn PMR5 gene or transcript, and the polynucleotide comprises at least 18 contiguous nucleotides that are essentially identical or essentially complementary to SEQ ID NO:7; (f) the plant is a tomato plant, the gene or the transcript is a tomato PMR5 gene or transcript, and the polynucleotide comprises at least 18 contiguous nucleotides that are essentially identical or essentially complementary to SEQ ID NO:2 or 10; (g) the plant is a wheat plant, the gene or the transcript is a wheat PMR5 gene or transcript, and the polynucleotide comprises at least 18 contiguous nucleotides that are essentially identical or essentially complementary to SEQ ID NO:5; or, (h) the plant is a rice plant, the gene or the transcript is a rice PMR5 gene or transcript, and the polynucleotide comprises at least 18 contiguous nucleotides that are essentially identical or essentially complementary to SEQ ID NO:9.

A further embodiment is directed to a plant comprising an exogenous polynucleotide that comprises at least 18 contiguous nucleotides that are essentially identical or essentially complementary to a PMR5 gene or transcript of the gene, wherein the exogenous polynucleotide is not operably linked to a promoter or to a viral vector, is not integrated into the chromosomal DNA of the plant, and is not found in a non-transgenic plant; and, wherein the plant exhibits an improvement in fungal disease resistance and/or nematode resistance that results from suppression of the PMR5 gene. In certain embodiments, plant further comprises an organosilicone compound or a component thereof. In certain embodiments, the polynucleotide is selected from the group consisting of SEQ ID NO: 12-19, 21-38, 53-127, and 128, or comprises at least 18 contiguous nucleotides that are essentially identical or essentially complementary to SEQ ID NO:1, 2, 3, 4, 5, 6, 7, 8, 9, 10, or 11. In certain embodiments (a) the plant is a soybean plant, the gene or the transcript is a soybean PMR5 gene or transcript, and the polynucleotide molecule is selected from the group consisting of SEQ ID NO:12-19, 57-127, and SEQ ID NO:128, or the polynucleotide comprises at least 18 contiguous nucleotides that are essentially identical or essentially complementary to SEQ ID NO:8 or 11; (b) the plant is a barley plant, the gene or the transcript is a barley PMR5 gene or transcript, and the polynucleotide molecule is selected from the group consisting of SEQ ID NO:21-37, and SEQ ID NO:38, or the polynucleotide comprises at least 18 contiguous nucleotides that are essentially identical or essentially complementary to SEQ ID NO:4; (c) the plant is a cucumber plant, the gene or the transcript is a cucumber PMR5 gene or transcript, and the polynucleotide comprises at least 18 contiguous nucleotides that are essentially identical or essentially complementary to SEQ ID NO:3 or 6; (d) the plant is a lettuce plant, the gene or the transcript is a lettuce PMR5 gene or transcript, and the polynucleotide comprises at least 18 contiguous nucleotides that are essentially identical or essentially complementary to SEQ ID NO:1; (e) the plant is a corn plant, the gene or the transcript is a corn PMR5 gene or transcript, and the polynucleotide comprises at least 18 contiguous nucleotides that are essentially identical or essentially complementary to SEQ ID NO:7; (f) the plant is a tomato plant, the gene or the transcript is a tomato PMR5 gene or transcript, and the polynucleotide comprises at least 18 contiguous nucleotides that are essentially identical or essentially complementary to SEQ ID NO:2 or 10; (g) the plant is a wheat plant, the gene or the transcript is a wheat PMR5 gene or transcript, and the polynucleotide comprises at least 18 contiguous nucleotides that are essentially identical or essentially complementary to SEQ ID NO:5; or, (h) the plant is a rice plant, the gene or the transcript is a rice PMR5 gene or transcript, and the polynucleotide comprises at least 18 contiguous nucleotides that are essentially identical or essentially complementary to SEQ ID NO:9.

An additional embodiment is directed to a plant part comprising an exogenous polynucleotide that comprises at least 18 contiguous nucleotides that are essentially identical or essentially complementary to a PMR5 gene or transcript of the gene, wherein the exogenous polynucleotide is not operably linked to a promoter or to a viral vector and is not found in a non-transgenic plant; and, wherein the plant part exhibits an improvement in fungal disease resistance and/or nematode resistance that results from suppression of the PMR5 gene. In certain embodiments, the polynucleotide is selected from the group consisting of SEQ ID NO: 12-19, 21-38, 53-127, and 128, or wherein the polynucleotide comprises at least 18 contiguous nucleotides that are essentially identical or essentially complementary to SEQ ID NO: 1, 2, 3, 4, 5, 6, 7, 8, 9, 10, or 11. In certain embodiments: (a) the plant is a soybean plant, the gene or the transcript is a soybean PMR5 gene or transcript, and the polynucleotide molecule is selected from the group consisting of SEQ ID NO:12-19, 57-127, and SEQ ID NO:128, or the polynucleotide comprises at least 18 contiguous nucleotides that are essentially identical or essentially complementary to SEQ ID NO:8 or 11; (b) the plant is a barley plant, the gene or the transcript is a barley PMR5 gene or transcript, and the polynucleotide molecule is selected from the group consisting of SEQ ID NO:21-37, and SEQ ID NO:38, or the polynucleotide comprises at least 18 contiguous nucleotides that are essentially identical or essentially complementary to SEQ ID NO:4; (c) the plant is a cucumber plant, the gene or the transcript is a cucumber PMR5 gene or transcript, and the polynucleotide comprises at least 18 contiguous nucleotides that are essentially identical or essentially complementary to SEQ ID NO:3, or 6, 53, 54, 55, or 56; (d) the plant is a lettuce plant, the gene or the transcript is a lettuce PMR5 gene or transcript, and the polynucleotide comprises at least 18 contiguous nucleotides that are essentially identical or essentially complementary to SEQ ID NO:1; (e) the plant is a corn plant, the gene or the transcript is a corn PMR5 gene or transcript, and the polynucleotide comprises at least 18 contiguous nucleotides that are essentially identical or essentially complementary to SEQ ID NO:7; (f) the plant is a tomato plant, the gene or the transcript is a tomato PMR5 gene or transcript, and the polynucleotide comprises at least 18 contiguous nucleotides that are essentially identical or essentially complementary to SEQ ID NO:2 or 10; (g) the plant is a wheat plant, the gene or the transcript is a wheat PMR5 gene or transcript, and the polynucleotide comprises at least 18 contiguous nucleotides that are essentially identical or essentially complementary to SEQ ID NO:5; or, (h) the plant is a rice plant, the gene or the transcript is a rice PMR5 gene or transcript, and the polynucleotide comprises at least 18 contiguous nucleotides that are essentially identical or essentially complementary to SEQ ID NO:9. In certain embodiments, the plant part is a flower, meristem, ovule, stem, tuber, fruit, anther, pollen, leaf, root, or seed. In certain embodiments, the plant part is a seed. Also provided are processed plant products obtained from any of the aforementioned plant parts, wherein the processed plant products exhibit an improved attribute relative to a processed plant product of an untreated control plant and wherein the improved attribute results from the improved fungal disease resistance and/or nematode resistance. In certain embodiments, the processed product is a meal, a pulp, a feed, or a food product. Another embodiment is directed to a plant that exhibits an improvement in fungal disease resistance and/or nematode resistance, wherein the plant was topically treated with a composition that comprises: (a) at least one polynucleotide that comprises at least 18 contiguous nucleotides that are essentially identical or essentially complementary to a PMR5 gene or to a transcript of the gene; and (b) a transfer agent; and, wherein the plant exhibits an improvement in fungal disease resistance and/or nematode resistance that results from suppression of the PMR5 gene.

Also provided herein are transgenic plants, plant parts, plant cells, and processed plant products containing a transgene comprising a heterologous promoter that is operably linked to a polynucleotide that comprises at least 18 contiguous nucleotides that are essentially identical or essentially complementary to a PMR5 gene or transcript of the PMR5 gene. Such transgenes can be integrated into the genome of the transgenic plant or provided in recombinant viral genomes that can be propagated in the plant. In certain embodiments, the transgene confers an improvement in fungal disease resistance and/or nematode resistance to the transgenic plants or plant parts that contain the transgene. In certain embodiments, the polynucleotide is selected from the group consisting of SEQ ID NO: 12-19, 21-38, 57-127, and 128, encodes an RNA comprising or consisting of SEQ ID NO: 53, 54, 55, 56, or their complements, encodes an RNA that is essentially identical or essentially complementary to SEQ ID NO: 53, 54, 55, 56, or comprises at least 18 contiguous nucleotides that are essentially identical or essentially complementary to SEQ ID NO: 1, 2, 3, 4, 5, 6, 7, 8, 9, 10, or 11. In certain embodiments: (a) the plant is a soybean plant, the gene or the transcript is a soybean PMR5 gene or transcript, and the polynucleotide molecule is selected from the group consisting of SEQ ID NO:12-19, 57-127, and SEQ ID NO:128, or the polynucleotide comprises at least 18 contiguous nucleotides that are essentially identical or essentially complementary to SEQ ID NO:8 or 11; (b) the plant is a barley plant, the gene or the transcript is a barley PMR5 gene or transcript, and the polynucleotide molecule is selected from the group consisting of SEQ ID NO:21-37, and SEQ ID NO:38, or the polynucleotide comprises at least 18 contiguous nucleotides that are essentially identical or essentially complementary to SEQ ID NO:4; (c) the plant is a cucumber plant, the gene or the transcript is a cucumber PMR5 gene or transcript, and the polynucleotide comprises at least 18 contiguous nucleotides that are essentially identical or essentially complementary to SEQ ID NO:3, 6, 53, 54, 55, or 56, or encodes an RNA comprising or consisting of SEQ ID NO: 53, 54, 55, or 56; (d) the plant is a lettuce plant, the gene or the transcript is a lettuce PMR5 gene or transcript, and the polynucleotide comprises at least 18 contiguous nucleotides that are essentially identical or essentially complementary to SEQ ID NO:1; (e) the plant is a corn plant, the gene or the transcript is a corn PMR5 gene or transcript, and the polynucleotide comprises at least 18 contiguous nucleotides that are essentially identical or essentially complementary to SEQ ID NO:7; (f) the plant is a tomato plant, the gene or the transcript is a tomato PMR5 gene or transcript, and the polynucleotide comprises at least 18 contiguous nucleotides that are essentially identical or essentially complementary to SEQ ID NO:2 or 10; (g) the plant is a wheat plant, the gene or the transcript is a wheat PMR5 gene or transcript, and the polynucleotide comprises at least 18 contiguous nucleotides that are essentially identical or essentially complementary to SEQ ID NO:5; or, (h) the plant is a rice plant, the gene or the transcript is a rice PMR5 gene or transcript, and the polynucleotide comprises at least 18 contiguous nucleotides that are essentially identical or essentially complementary to SEQ ID NO:9. In certain embodiments, the transgenic plant part is a flower, meristem, ovule, stem, tuber, fruit, anther, pollen, leaf, root, or seed. Processed plant products containing the transgene include, but are not limited to, a meal a pulp, a feed, or a food product obtainable from the transgenic plant parts. In certain embodiments, the processed plant products exhibit an improved attribute relative to a processed plant product of an untreated control plant and wherein the improved attribute results from the improved fungal disease resistance and/or nematode resistance conferred by the transgene. In certain embodiments, the processed product is a meal, a pulp, a feed, or a food product. Also provided herein are methods for obtaining transgenic plants exhibiting an improvement in fungal disease resistance and/or nematode resistance comprising the steps of introducing any of the aforementioned transgenes into the genome of a plant and selecting for a transgenic plant wherein expression of an endogenous PMR5 gene is suppressed, thereby obtaining a plant exhibiting an improvement in fungal disease resistance and/or nematode resistance. Also provided herein are methods for improving fungal disease resistance and/or nematode resistance in plants that comprise growing transgenic plants comprising any of the aforementioned transgenes wherein expression of an endogenous PMR5 is suppressed in the presence of fungi and/or nematodes, wherein fungal disease resistance and/or nematode resistance of the transgenic plants is improved in comparison to a control plant that lack a transgene that suppresses an endogenous PMR5 gene in the control plant.

### BRIEF DESCRIPTION OF THE DRAWINGS

FIG. 1 presents a bootstrapped phylogenetic tree of PMR5 proteins.

FIG. 2 presents barley powdery mildew disease control measurements in barley plants treated with various liquids. Certain results were obtained with liquids that contained certain nucleic acids as indicated in the labels along the X-axis.

FIG. 3 presents barley powdery mildew disease control measurements (percentage of the top half of the leaf area infected) in barley plants treated with various liquids. Certain results were obtained with liquids that contained certain individual oligonucleotides as indicated in the labels along the X-axis.

FIG. 4 presents barley powdery mildew disease control measurements (percentage of the leaf area infected) in barley plants treated with various liquids. Certain results were obtained with liquids that contained certain nucleic acids as indicated in the labels along the X-axis.

FIG. 5 presents Root Knot Nematode disease control measurements (number of RKN eggs/gram of root tissue) in soybean plants treated with various liquids. Certain results were obtained with liquids that contained certain nucleic acids as indicated in the labels along the X-axis.

### DETAILED DESCRIPTION

### I. Definitions

The following definitions and methods are provided to better define the present embodiments and to guide those of ordinary skill in the art in the practice of the embodiments disclosed in the present application. Unless otherwise noted, terms are to be understood according to conventional usage by those of ordinary skill in the relevant art.

Where a term is provided in the singular, the inventors also contemplate aspects described by the plural of that term.

As used herein, the terms “DNA,” “DNA molecule,” and “DNA polynucleotide molecule” refer to a single-stranded DNA or double-stranded DNA molecule of genomic or synthetic origin, such as, a polymer of deoxyribonucleotide bases or a DNA polynucleotide molecule.

As used herein, the terms “DNA sequence,” “DNA nucleotide sequence,” and “DNA polynucleotide sequence” refer to the nucleotide sequence of a DNA molecule.

As used herein, the term “gene” refers to any portion of a nucleic acid that provides for expression of a transcript or encodes a transcript. A “gene” thus includes, but is not limited to, a promoter region, 5′ untranslated regions, transcript encoding regions that can include intronic regions, and 3′ untranslated regions.

As used herein, the terms “RNA,” “RNA molecule,” and “RNA polynucleotide molecule” refer to a single-stranded RNA or double-stranded RNA molecule of genomic or synthetic origin, such as, a polymer of ribonucleotide bases that comprise single or double stranded regions.

Unless otherwise stated, nucleotide sequences in the text of this specification are given, when read from left to right, in the 5′ to 3′ direction. The nomenclature used herein is that required by Title 37 of the United States Code of Federal Regulations § 1.822 and set forth in the tables in WIPO Standard ST.25 (1998), Appendix 2, Tables 1 and 3.

As used herein, a “plant surface” refers to any exterior portion of a plant. Plant surfaces thus include, but are not limited to, the surfaces of flowers, stems, tubers, fruit, anthers, pollen, leaves, roots, or seeds. A plant surface can be on a portion of a plant that is attached to other portions of a plant or on a portion of a plant that is detached from the plant.

As used herein, the phrase “polynucleotide is not operably linked to a promoter” refers to a polynucleotide that is not covalently linked to a polynucleotide promoter sequence that is specifically recognized by either a DNA dependent RNA polymerase II protein or by a viral RNA dependent RNA polymerase in such a manner that the polynucleotide will be transcribed by the DNA dependent RNA polymerase II protein or viral RNA dependent RNA polymerase. A polynucleotide that is not operably linked to a promoter can be transcribed by a plant RNA dependent RNA polymerase.

As used herein, any polynucleotide sequences of SEQ ID NO: 12-19, 21-37, or 38, though displayed in the sequence listing in the form of ssDNA, encompass all other polynucleotide forms such as dsDNA equivalents, ssDNA equivalents, ssRNA equivalents, ssRNA complements, dsRNA, and ssDNA complements.

As used herein, a first nucleic-acid sequence is “operably” connected or “linked” with a second nucleic acid sequence when the first nucleic acid sequence is placed in a functional relationship with the second nucleic acid sequence. For instance, a promoter is operably linked to an RNA and/or protein-coding sequence if the promoter provides for transcription or expression of the RNA or coding sequence. Generally, operably linked DNA sequences are contiguous and, where necessary to join two protein-coding regions, are in the same reading frame.

As used herein, the phrase “organosilicone preparation” refers to a liquid comprising one or more organosilicone compounds, wherein the liquid or components contained therein, when combined with a polynucleotide in a composition that is topically applied to a target plant surface, enable the polynucleotide to enter a plant cell. Examples of organosilicone preparations include, but are not limited to, preparations marketed under the trade names “Silwet®” or “BREAK-THRU®” and preparations provided in Table 1. In certain embodiments, an organosilicone preparation can enable a polynucleotide to enter a plant cell in a manner permitting a polynucleotide suppression of target gene expression in the plant cell.

As used herein, the phrase “provides for an improvement in fungal disease resistance and/or nematode resistance” refers to any measurable increase in a plants resistance to fungal- and/or nematode-induced damage. In certain embodiments, an improvement in fungal disease resistance and/or nematode resistance in a plant or plant part can be determined in a comparison to a control plant or plant part that has not been treated with a composition comprising a polynucleotide and a transfer agent. When used in this context, a control plant is a plant that has not undergone treatment with polynucleotide and a transfer agent. Such control plants would include, but are not limited to, untreated plants or mock treated plants.

As used herein, the phrase “provides for a reduction”, when used in the context of a transcript or a protein in a plant or plant part, refers to any measurable decrease in the level of transcript or protein in a plant or plant part. In certain embodiments, a reduction of the level of a transcript or protein in a plant or plant part can be determined in a comparison to a control plant or plant part that has not been treated with a composition comprising a polynucleotide and a transfer agent. When used in this context, a control plant or plant part is a plant or plant part that has not undergone treatment with polynucleotide and a transfer agent. Such control plants or plant parts would include, but are not limited to, untreated or mock treated plants and plant parts.

As used herein, the phrase “wherein said plant does not comprise a transgene” refers to a plant that lacks either a DNA molecule comprising a promoter that is operably linked to a polynucleotide or a recombinant viral vector.

As used herein, the phrase “suppressing expression” or “suppression”, when used in the context of a gene, refers any measurable decrease in the amount and/or activity of a product encoded by the gene. Thus, expression of a gene can be suppressed when there is a reduction in levels of a transcript from the gene, a reduction in levels of a protein encoded by the gene, a reduction in the activity of the transcript from the gene, a reduction in the activity of a protein encoded by the gene, any one of the preceding conditions, or any combination of the preceding conditions. In this context, the activity of a transcript includes, but is not limited to, its ability to be translated into a protein and/or to exert any RNA-mediated biologic or biochemical effect. In this context, the activity of a protein includes, but is not limited to, its ability to exert any protein-mediated biologic or biochemical effect. In certain embodiments, a suppression of gene expression in a plant or plant part can be determined in a comparison of gene product levels or activities in a treated plant to a control plant or plant part that has not been treated with a composition comprising a polynucleotide and a transfer agent. When used in this context, a control plant or plant part is a plant or plant part that has not undergone treatment with polynucleotide and a transfer agent. Such control plants or plant parts would include, but are not limited to, untreated or mock treated plants and plant parts.

As used herein, the term “transcript” corresponds to any RNA that is produced from a gene by the process of transcription. A transcript of a gene can thus comprise a primary transcription product which can contain introns or can comprise a mature RNA that lacks introns.

As used herein, the term “liquid” refers to both homogeneous mixtures such as solutions and non-homogeneous mixtures such as suspensions, colloids, micelles, and emulsions.

### II. Overview

Provided herein are certain methods and polynucleotide compositions that can be applied to living plant cells/tissues to suppress expression of target genes and that provide improved fungal disease resistance and/or nematode resistance to a crop plant. Also provided herein are plants and plant parts exhibiting fungal disease resistance and/or nematode resistance as well as processed products of such plants or plant parts. The compositions may be topically applied to the surface of a plant, such as to the surface of a leaf, and include a transfer agent. Aspects of the method can be applied to various crops, for example, including but not limited to: i) row crop plants including, but are not limited to, corn, barley, sorghum, soybean, cotton, canola, sugar beet, alfalfa, sugarcane, rice, and wheat; ii) vegetable plants including, but not limited to, tomato, potato, sweet pepper, hot pepper, melon, watermelon, cucumber, eggplant, cauliflower, broccoli, lettuce, spinach, onion, peas, carrots, sweet corn, Chinese cabbage, leek, fennel, pumpkin, squash or gourd, radish, Brussels sprouts, tomatillo, garden beans, dry beans, or okra; iii) culinary plants including, but not limited to, basil, parsley, coffee, or tea; iv) fruit plants including but not limited to apple, pear, cherry, peach, plum, apricot, banana, plantain, table grape, wine grape, citrus, avocado, mango, or berry; v) a tree grown for ornamental or commercial use, including, but not limited to, a fruit or nut tree; or, vi) an ornamental plant (e. g., an ornamental flowering plant or shrub or turf grass). The methods and compositions provided herein can also be applied to plants produced by a cutting, cloning, or grafting process (i. e., a plant not grown from a seed) that include fruit trees and plants. Fruit trees produced by such processes include, but are not limited to, citrus and apple trees. Plants produced by such processes include, but are not limited to, avocados, tomatoes, eggplant, cucumber, melons, watermelons, and grapes as well as various ornamental plants.

Without being bound by theory, the compositions and methods of the present embodiments are believed to operate through one or more of the several natural cellular pathways involved in RNA-mediated gene suppression as generally described in Brodersen and Voinnet (2006), Trends Genetics, 22:268-280; Tomari and Zamore (2005) Genes & Dev., 19:517-529; Vaucheret (2006) Genes Dev., 20:759-771; Meins et al. (2005) Annu. Rev. Cell Dev. Biol., 21:297-318; and Jones-Rhoades et al. (2006) Annu. Rev. Plant Biol., 57:19-53. RNA-mediated gene suppression generally involves a double-stranded RNA (dsRNA) intermediate that is formed intra-molecularly within a single RNA molecule or inter-molecularly between two RNA molecules. This longer dsRNA intermediate is processed by a ribonuclease of the RNAase III family (Dicer or Dicer-like ribonuclease) to one or more shorter double-stranded RNAs, one strand of which is incorporated into the RNA-induced silencing complex (“RISC”). For example, the siRNA pathway involves the cleavage of a longer double-stranded RNA intermediate to small interfering RNAs (“siRNAs”). The size of siRNAs is believed to range from about 19 to about 25 base pairs, but the most common classes of siRNAs in plants include those containing 21 to 24 base pairs (See, Hamilton et al. (2002) EMBO J., 21:4671-4679).

### Polynucleotides

As used herein, “polynucleotide” refers to a DNA or RNA molecule containing multiple nucleotides and generally refers both to “oligonucleotides” (a polynucleotide molecule of 18-25 nucleotides in length) and longer polynucleotides of 26 or more nucleotides. Embodiments include compositions including oligonucleotides having a length of 18-25 nucleotides (18-mers, 19-mers, 20-mers, 21-mers, 22-mers, 23-mers, 24-mers, or 25-mers), or medium-length polynucleotides having a length of 26 or more nucleotides (polynucleotides of 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, or about 300 nucleotides), or long polynucleotides having a length greater than about 300 nucleotides (e. g., polynucleotides of between about 300 to about 400 nucleotides, between about 400 to about 500 nucleotides, between about 500 to about 600 nucleotides, between about 600 to about 700 nucleotides, between about 700 to about 800 nucleotides, between about 800 to about 900 nucleotides, between about 900 to about 1000 nucleotides, between about 300 to about 500 nucleotides, between about 300 to about 600 nucleotides, between about 300 to about 700 nucleotides, between about 300 to about 800 nucleotides, between about 300 to about 900 nucleotides, or about 1000 nucleotides in length, or even greater than about 1000 nucleotides in length, for example up to the entire length of a target gene including coding or non-coding or both coding and non-coding portions of the target gene). Where a polynucleotide is double-stranded, its length can be similarly described in terms of base pairs.

Polynucleotide compositions used in the various embodiments include compositions including oligonucleotides, polynucleotides, or a mixture of both, including: RNA or DNA or RNA/DNA hybrids or chemically modified oligonucleotides or polynucleotides or a mixture thereof. In certain embodiments, the polynucleotide may be a combination of ribonucleotides and deoxyribonucleotides, for example, synthetic polynucleotides consisting mainly of ribonucleotides but with one or more terminal deoxyribonucleotides or synthetic polynucleotides consisting mainly of deoxyribonucleotides but with one or more terminal dideoxyribonucleotides. In certain embodiments, the polynucleotide includes non-canonical nucleotides such as inosine, thiouridine, or pseudouridine. In certain embodiments, the polynucleotide includes chemically modified nucleotides. Examples of chemically modified oligonucleotides or polynucleotides are well known in the art; see, for example, U.S. Patent Publication 2011/0171287, U.S. Patent Publication 2011/0171176, U.S. Patent Publication 2011/0152353, U.S. Patent Publication 2011/0152346, and U.S. Patent Publication 2011/0160082, which are herein incorporated by reference. Illustrative examples include, but are not limited to, the naturally occurring phosphodiester backbone of an oligonucleotide or polynucleotide which can be partially or completely modified with phosphorothioate, phosphorodithioate, or methylphosphonate internucleotide linkage modifications, modified nucleoside bases or modified sugars can be used in oligonucleotide or polynucleotide synthesis, and oligonucleotides or polynucleotides can be labeled with a fluorescent moiety (e.g., fluorescein or rhodamine) or other label (e.g., biotin).

Polynucleotides can be single- or double-stranded RNA, single- or double-stranded DNA, double-stranded DNA/RNA hybrids, and modified analogues thereof. In certain embodiments, the polynucleotides that provide single-stranded RNA in the plant cell may be: (a) a single-stranded RNA molecule (ssRNA), (b) a single-stranded RNA molecule that self-hybridizes to form a double-stranded RNA molecule, (c) a double-stranded RNA molecule (dsRNA), (d) a single-stranded DNA molecule (ssDNA), (e) a single-stranded DNA molecule that self-hybridizes to form a double-stranded DNA molecule, (f) a single-stranded DNA molecule including a modified Pol III gene that is transcribed to an RNA molecule, (g) a double-stranded DNA molecule (dsDNA), (h) a double-stranded DNA molecule including a modified Pol III gene that is transcribed to an RNA molecule, and (i) a double-stranded, hybridized RNA/DNA molecule, or combinations thereof. In certain embodiments, these polynucleotides can comprise both ribonucleic acid residues and deoxyribonucleic acid residues. In certain embodiments, these polynucleotides include chemically modified nucleotides or non-canonical nucleotides. In certain embodiments of the methods, the polynucleotides include double-stranded DNA formed by intramolecular hybridization, double-stranded DNA formed by intermolecular hybridization, double-stranded RNA formed by intramolecular hybridization, or double-stranded RNA formed by intermolecular hybridization. In certain embodiments where the polynucleotide is a dsRNA, the anti-sense strand will comprise at least 18 nucleotides that are essentially complementary to the target gene. In certain embodiments the polynucleotides include single-stranded DNA or single-stranded RNA that self-hybridizes to form a hairpin structure having an at least partially double-stranded structure including at least one segment that will hybridize to RNA transcribed from the gene targeted for suppression. Not intending to be bound by any mechanism, it is believed that such polynucleotides are or will produce single-stranded RNA with at least one segment that will hybridize to RNA transcribed from the gene targeted for suppression. In certain embodiments, the polynucleotides can be operably linked to a promoter—generally a promoter functional in a plant, for example, a pol II promoter, a pol III promoter, a pol IV promoter, or a pol V promoter.

The polynucleotide molecules are designed to modulate expression by inducing regulation or suppression of an endogenous gene in a plant and are designed to have a nucleotide sequence essentially identical or essentially complementary to the nucleotide sequence of an endogenous gene of a plant or to the sequence of RNA transcribed from an endogenous gene of a plant, which can be coding sequence or non-coding sequence. These effective polynucleotide molecules that modulate expression are referred to herein as “a trigger, or triggers”. By “essentially identical” or “essentially complementary” it is meant that the trigger polynucleotides (or at least one strand of a double-stranded polynucleotide) have sufficient identity or complementarity to the endogenous gene or to the RNA transcribed from the endogenous gene (e.g. the transcript) to suppress expression of the endogenous gene (e.g. to effect a reduction in levels or activity of the gene transcript and/or encoded protein). Polynucleotides of the methods and compositions provided herein need not have 100 percent identity to a complementarity to the endogenous gene or to the RNA transcribed from the endogenous gene (i.e. the transcript) to suppress expression of the endogenous gene (i.e. to effect a reduction in levels or activity of the gene transcript or encoded protein). Thus, in certain embodiments, the polynucleotide or a portion thereof is designed to be essentially identical to, or essentially complementary to, a sequence of at least 18 or 19 contiguous nucleotides in either the target gene or messenger RNA transcribed from the target gene (e.g. the transcript). In certain embodiments, an “essentially identical” polynucleotide has 100 percent sequence identity or at least about 83, 84, 85, 86, 87, 88, 89, 90, 91, 92, 93, 94, 95, 96, 97, 98, or 99 percent sequence identity when compared to the sequence of 18 or more contiguous nucleotides in either the endogenous target gene or to an RNA transcribed from the target gene (e.g. the transcript). In certain embodiments, an “essentially complementary” polynucleotide has 100 percent sequence complementarity or at least about 83, 84, 85, 86, 87, 88, 89, 90, 91, 92, 93, 94, 95, 96, 97, 98, or 99 percent sequence complementarity when compared to the sequence of 18 or more contiguous nucleotides in either the target gene or RNA transcribed from the target gene.

In certain embodiments, polynucleotides used in the methods and compositions provided herein can be essentially identical or essentially complementary to any of: i) conserved regions of PMR5 genes of both monocot and dicot plants; ii) conserved regions of PMR5 genes of monocot plants; or iii) conserved regions of PMR5 genes of dicot plants. Such polynucleotides that are essentially identical or essentially complementary to such conserved regions can be used to improve fungal disease resistance and/or nematode disease resistance by suppressing expression of PMR5 genes in any of: i) both dicot and monocot plants, including, but not limited to, corn, barley, wheat, sorghum, rice, cucumber, pea, Medicago sp., soybean, pepper, tomato, and grape; ii) monocot plants, including, but not limited to, corn, barley, wheat, sorghum, and rice, and; or iii) dicot plants, including, but not limited to, cucumber, pea, Medicago sp., soybean, pepper, tomato, and grape.

Polynucleotides containing mismatches to the target gene or transcript can thus be used in certain embodiments of the compositions and methods provided herein. In certain embodiments, a polynucleotide can comprise at least 19 contiguous nucleotides that are essentially identical or essentially complementary to said gene or said transcript or comprises at least 19 contiguous nucleotides that are essentially identical or essentially complementary to the target gene or target gene transcript. In certain embodiments, a polynucleotide of 19 continuous nucleotides that is essentially identical or essentially complementary to the endogenous target gene or to RNA transcribed from the target gene (e.g. the transcript) can have 1 or 2 mismatches to the target gene or transcript. In certain embodiments, a polynucleotide of 20 or more nucleotides that contains a contiguous 19 nucleotide span of identity or complementarity to the endogenous target gene or to an RNA transcribed from the target gene can have 1 or 2 mismatches to the target gene or transcript. In certain embodiments, a polynucleotide of 21 continuous nucleotides that is essentially identical or essentially complementary to the endogenous target gene or to RNA transcribed from the target gene (e.g. the transcript) can have 1, 2, or 3 mismatches to the target gene or transcript. In certain embodiments, a polynucleotide of 22 or more nucleotides that contains a contiguous 21 nucleotide span of identity or complementarity to the endogenous target gene or to an RNA transcribed from the target gene can have 1, 2, or 3 mismatches to the target gene or transcript. In designing polynucleotides with mismatches to an endogenous target gene or to an RNA transcribed from the target gene, mismatches of certain types and at certain positions that are more likely to be tolerated can be used. In certain embodiments, mismatches formed between adenine and cytosine or guanosine and uracil residues are used as described by Du et al. Nucleic Acids Research, 2005, Vol. 33, No. 5 1671-1677. In certain embodiments, mismatches in 19 base pair overlap regions can be at the low tolerance positions 5, 7, 8 or 11 (from the 5′ end of a 19 nucleotide target) with well tolerated nucleotide mismatch residues, at medium tolerance positions 3, 4, and 12-17, and/or at the high tolerance nucleotide positions at either end of the region of complementarity (i.e. positions 1, 2, 18, and 19) as described by Du et al. Nucleic Acids Research, 2005, Vol. 33, No. 5 1671-1677. It is further anticipated that tolerated mismatches can be empirically determined in assays where the polynucleotide is applied to the plants via the methods provided herein and the treated plants assayed for suppression of PMR5 expression or appearance of fungal disease resistance and/or nematode resistance.

In certain embodiments, polynucleotide molecules are designed to have 100 percent sequence identity with or complementarity to one allele or one family member of a given target gene coding or non-coding sequence of a PMR5 target gene. In other embodiments, the polynucleotide molecules are designed to have 100 percent sequence identity with or complementarity to multiple alleles or family members of a given PMR5 target gene. In certain embodiments, the polynucleotide can thus comprise at least 18 contiguous nucleotides that are identical or complementary to SEQ ID NO: 1-19, 21-38, or 53-129. In certain embodiments, the polynucleotide comprises at least 18 contiguous nucleotides that are essentially identical or essentially complementary to SEQ ID NO: 1-19, 21-38, or 53-128.

In certain embodiments, polynucleotide compositions and methods provided herein typically effect regulation or modulation (e. g., suppression) of gene expression during a period during the life of the treated plant of at least 1 week or longer and typically in systemic fashion. For instance, within days of treating a plant leaf with a polynucleotide composition as described herein, primary and transitive siRNAs can be detected in other leaves lateral to and above the treated leaf and in apical tissue. In certain embodiments, methods of systemically suppressing expression of a gene in a plant, the methods comprising treating said plant with a composition comprising at least one polynucleotide and a transfer agent, wherein said polynucleotide comprises at least 18 or at least 19 contiguous nucleotides that are essentially identical or essentially complementary to a gene or a transcript encoding a PMR5 gene of the plant are provided, whereby expression of the gene in said plant or progeny thereof is systemically suppressed in comparison to a control plant that has not been treated with the composition.

Compositions used to suppress a target gene can comprise one or more polynucleotides that are essentially identical or essentially complementary to multiple genes, or to multiple segments of one or more genes. In certain embodiments, compositions used to suppress a target gene can comprise one or more polynucleotides that are essentially identical or essentially complementary to multiple consecutive segments of a target gene, multiple non-consecutive segments of a target gene, multiple alleles of a target gene, or multiple target genes from one or more species.

In certain embodiments, the polynucleotide includes two or more copies of a nucleotide sequence (of 18 or more nucleotides) where the copies are arranged in tandem fashion. In another embodiment, the polynucleotide includes two or more copies of a nucleotide sequence (of 18 or more nucleotides) where the copies are arranged in inverted repeat fashion (forming an at least partially self-complementary strand). The polynucleotide can include both tandem and inverted-repeat copies. Whether arranged in tandem or inverted repeat fashion, each copy can be directly contiguous to the next, or pairs of copies can be separated by an optional spacer of one or more nucleotides. The optional spacer can be unrelated sequence (i. e., not essentially identical to or essentially complementary to the copies, nor essentially identical to, or essentially complementary to, a sequence of 18 or more contiguous nucleotides of the endogenous target gene or RNA transcribed from the endogenous target gene). Alternatively the optional spacer can include sequence that is complementary to a segment of the endogenous target gene adjacent to the segment that is targeted by the copies. In certain embodiments, the polynucleotide includes two copies of a nucleotide sequence of between about 20 to about 30 nucleotides, where the two copies are separated by a spacer no longer than the length of the nucleotide sequence.

### Tiling

Polynucleotide trigger molecules can be identified by “tiling” gene targets in random length fragments, e.g. 200-300 polynucleotides in length, with partially overlapping regions, e.g. 25 or so nucleotide overlapping regions along the length of the target gene. Multiple gene target sequences can be aligned and polynucleotide sequence regions with homology in common are identified as potential trigger molecules for multiple targets. Multiple target sequences can be aligned and sequence regions with poor homology are identified as potential trigger molecules for selectively distinguishing targets. To selectively suppress a single gene, trigger sequences may be chosen from regions that are unique to the target gene either from the transcribed region or the non-coding regions, e.g., promoter regions, 3′ untranslated regions, introns and the like.

Polynucleotides fragments are designed along the length of the full length coding and untranslated regions of a PMR5 gene or family member as contiguous overlapping fragments of 200-300 polynucleotides in length or fragment lengths representing a percentage of the target gene. These fragments are applied topically (as sense or anti-sense ssDNA or ssRNA, dsRNA, or dsDNA) to determine the relative effectiveness in providing the yield/quality phenotype. Fragments providing the desired activity may be further subdivided into 50-60 polynucleotide fragments which are evaluated for providing the yield/quality phenotype. The 50-60 base fragments with the desired activity may then be further subdivided into 19-30 base fragments which are evaluated for providing the yield/quality phenotype. Once relative effectiveness is determined, the fragments are utilized singly, or in combination in one or more pools to determine effective trigger composition or mixture of trigger polynucleotides for providing the yield/quality phenotype.

Coding and/or non-coding sequences of gene families in the crop of interest are aligned and 200-300 polynucleotide fragments from the least homologous regions amongst the aligned sequences are evaluated using topically applied polynucleotides (as sense or anti-sense ssDNA or ssRNA, dsRNA, or dsDNA) to determine their relative effectiveness in providing the yield/quality phenotype. The effective segments are further subdivided into 50-60 polynucleotide fragments, prioritized by least homology, and reevaluated using topically applied polynucleotides. The effective 50-60 polynucleotide fragments are subdivided into 19-30 polynucleotide fragments, prioritized by least homology, and again evaluated for induction of the yield/quality phenotype. Once relative effectiveness is determined, the fragments are utilized singly, or again evaluated in combination with one or more other fragments to determine the trigger composition or mixture of trigger polynucleotides for providing the yield/quality phenotype.

Coding and/or non-coding sequences of gene families in the crop of interest are aligned and 200-300 polynucleotide fragments from the most homologous regions amongst the aligned sequences are evaluated using topically applied polynucleotides (as sense or anti-sense ssDNA or ssRNA, dsRNA, or dsDNA) to determine their relative effectiveness in inducing the yield/quality phenotype. The effective segments are subdivided into 50-60 polynucleotide fragments, prioritized by most homology, and reevaluated using topically applied polynucleotides. The effective 50-60 polynucleotide fragments are subdivided into 19-30 polynucleotide fragments, prioritized by most homology, and again evaluated for induction of the yield/quality phenotype. Once relative effectiveness is determined, the fragments may be utilized singly, or in combination with one or more other fragments to determine the trigger composition or mixture of trigger polynucleotides for providing the yield/quality phenotype.

Also, provided herein are methods for identifying a preferred polynucleotide for improving fungal disease and/or nematode resistance in a plant. Populations of candidate polynucleotides that are essentially identical or essentially complementary to a PMR5 gene or transcript of the gene can be generated by a variety of approaches, including but not limited to, any of the tiling, least homology, or most homology approaches provided herein. Such populations of polynucleotides can also be generated or obtained from any of the polynucleotides or genes provided herewith in SEQ ID NO:1-19 or 21-38 or 53-128. Such populations of polynucleotides can also be generated or obtained from any genes that are orthologous to the genes provided herewith in SEQ ID NO:1-11. Such populations of polynucleotides can also be generated or obtained from any genes that encode proteins that are orthologous to a protein of Table 3 (SEQ ID NO:41-48, or 49). Any of the aforementioned populations of polynucleotides can also be selected by testing candidate polynucleotides for suppression of PMR5 via Viral Induced Gene Silencing (VIGS) methods. In certain embodiments, selection of polynucleotides that can potentially provide PMR5 gene suppression when applied to plants with a transfer agent can be effected by Viral Induced Gene Silencing (VIGS) methods. In general, a candidate PMR5 suppression sequence is tested by insertion of that sequence into a cloned viral genome that can be introduced into a target plant or target plant cell to effect PMR5 suppression. Various methods and vectors used for suppression of gene targets by VIGS can be adapted for use in testing for suppression of PMR5 genes by use of appropriate candidate PMR5 suppression sequences disclosed herein. VIGS methods and vectors that can be used for performing VIGS in dicot plants include, but are not limited to, those disclosed in U.S. Pat. Nos. 5,922,602, 6,635,805, 6,369,296, and 7,229,829, which are each incorporated herein by reference in their entireties with respect to their disclosure of VIGS vectors and methods. VIGS methods and vectors that can be used for performing VIGS in monocot plants include, but are not limited to, those disclosed in U.S. Pat. No. 6,800,748, which is incorporated herein by reference in its entirety with respect to its disclosure of VIGS vectors and methods. Candidate polynucleotide sequences can be tested for PMR5 suppression with VIGS vectors and methods based on cloned Hordeivirus (including, but not limited to, barley stripe mosaic virus (“BSMV”), poa semilatent virus (“PSLV”), lychnis ringspot virus (“LRSV”), and anthoxanthum latent blanching virus (“ALBV”)), tobacco mosaic virus (TMV), Cucumber Green Mottle Mosaic virus watermelon strain (CGMMV-W); Brome Mosaic virus (BMV), Potyvirus (including, but not limited to, Rice Necrosis virus, and Potato Virus Y (PVY)), Rice tungro bacilliform virus (RTBV) and Geminivirus (including, but not limited to, Tomato Golden Mosaic Virus (ToGMV)) genomes. Useful ToGMV vectors that can be used are described by Revington et al. (Plant Cell. 1989 October; 1(10): 985-992). Useful methods for effecting VIGS via vacuum infiltration mediated agroinfection methods described in Yan et al. (Plant Cell Rep (2012) 31:1713-1722) can be adapted for testing candidate PMR5 suppression sequences in Agrobacterium-based VIGS vectors. Such polynucleotides can be topically applied to a surface of plants in a composition comprising at least one polynucleotide from said population and a transfer agent to obtain treated plants. Treated plants that exhibit suppression of the PMR5 gene and/or exhibit an improvement fungal disease and/or nematode resistance are identified, thus identifying a preferred polynucleotide that improves improving fungal disease and/or nematode resistance in a plant. Suppression of the gene can be determined by any assay for the levels and/or activity of a gene product (i.e. transcript or protein). Suitable assays for transcripts include, but are not limited to, semi-quantitative or quantitative reverse transcriptase PCR® (qRT-PCR) assays. Suitable assays for proteins include, but are not limited to, semi-quantitative or quantitaive immunoassays, biochemical activity assays, or biological activity assays. In certain embodiments, the polynucleotides can be applied alone. In other embodiments, the polynucleotides can be applied in pools of multiple polynucleotides. When a pool of polynucleotides provides for suppression of the PMR5 gene and/or an improvement in fungal disease resistance and/or nematode disease resistance are identified, the pool can be de-replicated and retested as necessary or desired to identify one or more preferred polynucleotide(s) that improves fungal disease resistance and/or nematode disease resistance in a plant.

Methods of making polynucleotides are well known in the art. Such methods of making polynucleotides can include in vivo biosynthesis, in vitro enzymatic synthesis, or chemical synthesis. In certain embodiments, RNA molecules can be made by either in vivo or in vitro synthesis from DNA templates where a suitable promoter is operably linked to the polynucleotide and a suitable DNA—dependent RNA polymerase is provided. DNA—dependent RNA polymerases include, but are not limited to, E. coli or other bacterial RNA polymerases as well as the bacteriophage RNA polymerases such as the T7, T3, and SP6 RNA polymerases. Commercial preparation of oligonucleotides often provides two deoxyribonucleotides on the 3′ end of the sense strand. Long polynucleotide molecules can be synthesized from commercially available kits, for example, kits from Applied Biosystems/Ambion (Austin, Tex.) have DNA ligated on the 5′ end that encodes a bacteriophage T7 polymerase promoter that makes RNA strands that can be assembled into a dsRNA. Alternatively, dsRNA molecules can be produced from expression cassettes in bacterial cells that have regulated or deficient RNase III enzyme activity. Long polynucleotide molecules can also be assembled from multiple RNA or DNA fragments. In some embodiments design parameters such as Reynolds score (Reynolds et al. Nature Biotechnology 22, 326-330 (2004) and Tuschl rules (Pei and Tuschl, Nature Methods 3(9): 670-676, 2006) are known in the art and are used in selecting polynucleotide sequences effective in gene silencing. In some embodiments random design or empirical selection of polynucleotide sequences is used in selecting polynucleotide sequences effective in gene silencing. In some embodiments the sequence of a polynucleotide is screened against the genomic DNA of the intended plant to minimize unintentional silencing of other genes.

While there is no upper limit on the concentrations and dosages of polynucleotide molecules that can be useful in the methods and compositions provided herein, lower effective concentrations and dosages will generally be sought for efficiency. The concentrations can be adjusted in consideration of the volume of spray or treatment applied to plant leaves or other plant part surfaces, such as flower petals, stems, tubers, fruit, anthers, pollen, leaves, roots, or seeds. In one embodiment, a useful treatment for herbaceous plants using 25-mer polynucleotide molecules is about 1 nanomole (nmol) of polynucleotide molecules per plant, for example, from about 0.05 to 1 nmol polynucleotides per plant. Other embodiments for herbaceous plants include useful ranges of about 0.05 to about 100 nmol, or about 0.1 to about 20 nmol, or about 1 nmol to about 10 nmol of polynucleotides per plant. In certain embodiments, about 40 to about 50 nmol of a ssDNA polynucleotide are applied. In certain embodiments, about 0.5 nmol to about 2 nmol of a dsRNA is applied. In certain embodiments, a composition containing about 0.5 to about 2.0 mg/mL, or about 0.14 mg/mL of dsRNA or ssDNA (21-mer) is applied. In certain embodiments, a composition of about 0.5 to about 1.5 mg/mL of a long dsRNA polynucleotide (i.e. about 50 to about 200 or more nucleotides) is applied. In certain embodiments, about 1 nmol to about 5 nmol of a dsRNA is applied to a plant. In certain embodiments, the polynucleotide composition as topically applied to the plant contains the at least one polynucleotide at a concentration of about 0.01 to about 10 milligrams per milliliter, or about 0.05 to about 2 milligrams per milliliter, or about 0.1 to about 2 milligrams per milliliter. In certain embodiments, a composition of about 0.5 to about 1.5 mg/mL of a long dsRNA polynucleotide (i.e. about 50 to about 200 or more nucleotides) is applied. Very large plants, trees, or vines may require correspondingly larger amounts of polynucleotides. When using long dsRNA molecules that can be processed into multiple oligonucleotides, lower concentrations can be used. To illustrate embodiments, the factor 1×, when applied to oligonucleotide molecules is arbitrarily used to denote a treatment of 0.8 nmol of polynucleotide molecule per plant; 10×, 8 nmol of polynucleotide molecule per plant; and 100×, 80 nmol of polynucleotide molecule per plant.

The polynucleotide compositions described herein are useful in compositions, such as liquids that comprise polynucleotide molecules, alone or in combination with other components either in the same liquid or in separately applied liquids that provide a transfer agent. As used herein, a transfer agent is an agent that, when combined with a polynucleotide in a composition that is topically applied to a target plant surface, enables the polynucleotide to enter a plant cell. In certain embodiments, a transfer agent is an agent that conditions the surface of plant tissue, e. g., seeds, leaves, stems, roots, flowers, or fruits, to permeation by the polynucleotide molecules into plant cells. The transfer of polynucleotides into plant cells can be facilitated by the prior or contemporaneous application of a polynucleotide-transferring agent to the plant tissue. In some embodiments the transferring agent is applied subsequent to the application of the polynucleotide composition. The polynucleotide transfer agent enables a pathway for polynucleotides through cuticle wax barriers, stomata and/or cell wall or membrane barriers into plant cells. Suitable transfer agents to facilitate transfer of the polynucleotide into a plant cell include agents that increase permeability of the exterior of the plant or that increase permeability of plant cells to oligonucleotides or polynucleotides. Such agents to facilitate transfer of the composition into a plant cell include a chemical agent, or a physical agent, or combinations thereof. Chemical agents for conditioning or transfer include (a) surfactants, (b) an organic solvent or an aqueous solution or aqueous mixtures of organic solvents, (c) oxidizing agents, (d) acids, (e) bases, (f) oils, (g) enzymes, or combinations thereof. Embodiments of the method can optionally include an incubation step, a neutralization step (e.g., to neutralize an acid, base, or oxidizing agent, or to inactivate an enzyme), a rinsing step, or combinations thereof. Embodiments of agents or treatments for conditioning of a plant to permeation by polynucleotides include emulsions, reverse emulsions, liposomes, and other micellar-like compositions. Embodiments of agents or treatments for conditioning of a plant to permeation by polynucleotides include counter-ions or other molecules that are known to associate with nucleic acid molecules, e. g., inorganic ammonium ions, alkyl ammonium ions, lithium ions, polyamines such as spermine, spermidine, or putrescine, and other cations. Organic solvents useful in conditioning a plant to permeation by polynucleotides include DMSO, DMF, pyridine, N-pyrrolidine, hexamethylphosphoramide, acetonitrile, dioxane, polypropylene glycol, other solvents miscible with water or that will dissolve phosphonucleotides in non-aqueous systems (such as is used in synthetic reactions). Naturally derived or synthetic oils with or without surfactants or emulsifiers can be used, e. g., plant-sourced oils, crop oils (such as those listed in the 9^{th} Compendium of Herbicide Adjuvants, publicly available on the worldwide web (internet) at herbicide.adjuvants.com can be used, e. g., paraffinic oils, polyol fatty acid esters, or oils with short-chain molecules modified with amides or polyamines such as polyethyleneimine or N-pyrrolidine. Transfer agents include, but are not limited to, organosilicone preparations.

In certain embodiments, an organosilicone preparation that is commercially available as Silwet® L-77 surfactant having CAS Number 27306-78-1 and EPA Number: CAL.REG.NO. 5905-50073-AA, and currently available from Momentive Performance Materials, Albany, N.Y. can be used to prepare a polynucleotide composition. In certain embodiments where a Silwet L-77 organosilicone preparation is used as a pre-spray treatment of plant leaves or other plant surfaces, freshly made concentrations in the range of about 0.015 to about 2 percent by weight (wt percent) (e. g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) are efficacious in preparing a leaf or other plant surface for transfer of polynucleotide molecules into plant cells from a topical application on the surface. In certain embodiments of the methods and compositions provided herein, a composition that comprises a polynucleotide molecule and an organosilicone preparation comprising Silwet L-77 in the range of about 0.015 to about 2 percent by weight (wt percent) (e. g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) is used or provided. In certain embodiments of the methods and compositions provided herein, a composition that comprises a polynucleotide molecule and an organosilicone preparation comprising Silwet L-77 in the range of about 0.3 to about 1 percent by weight (wt percent) or about 0.5 to about 1% by weight (wt percent) is used or provided.

In certain embodiments, any of the commercially available organosilicone preparations provided in the following Table 1 can be used as transfer agents in a polynucleotide composition. In certain embodiments where an organosilicone preparation of Table 1 is used as a pre-spray treatment of plant leaves or other surfaces, freshly made concentrations in the range of about 0.015 to about 2 percent by weight (wt percent) (e. g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) are efficacious in preparing a leaf or other plant surface for transfer of polynucleotide molecules into plant cells from a topical application on the surface. In certain embodiments of the methods and compositions provided herein, a composition that comprises a polynucleotide molecule and an organosilicone preparation of Table 1 in the range of about 0.015 to about 2 percent by weight (wt percent) (e. g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) is used or provided.

TABLE 1
Examples of organosilicone preparations

| Name | CAS number | Manufacturer ^{1, 2} |
|---|---|---|
| BREAK-THRU ® S 321 | na | Evonik Industries AG |
| BREAK-THRU ® S 200 | 67674-67-3 | Evonik Industries AG |
| BREAK-THRU ® OE 441 | 68937-55-3 | Evonik Industries AG |
| BREAK-THRU ® S 278 | 27306-78-1 | Evonik Goldschmidt |
| BREAK-THRU ® S 243 | na | Evonik Industries AG |
| Silwet ® L-77 | 27306-78-1 | Momentive Performance |
| | | Materials |
| Silwet ® HS 429 | na | Momentive Performance |
| | | Materials |
| Silwet ® HS 312 | na | Momentive Performance |
| | | Materials |
| BREAK-THRU ® S 233 | 134180-76-0 | Evonik Industries AG |
| Silwet ® HS 508 | | Momentive Performance |
| | | Materials |
| Silwet ® HS 604 | | Momentive Performance |
| | | Materials |

¹ Evonik Industries AG, Essen, Germany
² Momentive Performance Materials, Albany, New York

Organosilicone preparations used in the methods and compositions provided herein can comprise one or more effective organosilicone compounds. As used herein, the phrase “effective organosilicone compound” is used to describe any organosilicone compound that is found in an organosilicone preparation that enables a polynucleotide to enter a plant cell. In certain embodiments, an effective organosilicone compound can enable a polynucleotide to enter a plant cell in a manner permitting a polynucleotide mediated suppression of a target gene expression in the plant cell. In general, effective organosilicone compounds include, but are not limited to, compounds that can comprise: i) a trisiloxane head group that is covalently linked to, ii) an alkyl linker including, but not limited to, an n-propyl linker, that is covalently linked to, iii) a poly glycol chain, that is covalently linked to, iv) a terminal group. Trisiloxane head groups of such effective organosilicone compounds include, but are not limited to, heptamethyltrisiloxane. Alkyl linkers can include, but are not limited to, an n-propyl linker. Poly glycol chains include, but are not limited to, polyethylene glycol or polypropylene glycol. Poly glycol chains can comprise a mixture that provides an average chain length “n” of about “7.5”. In certain embodiments, the average chain length “n” can vary from about 5 to about 14. Terminal groups can include, but are not limited to, alkyl groups such as a methyl group. Effective organosilicone compounds are believed to include, but are not limited to, trisiloxane ethoxylate surfactants or polyalkylene oxide modified heptamethyl trisiloxane.

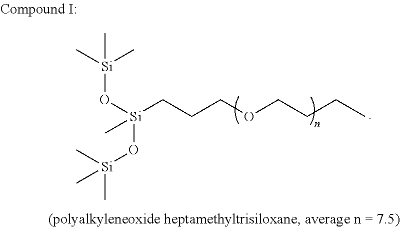

One organosilicone compound believed to be ineffective comprises the formula

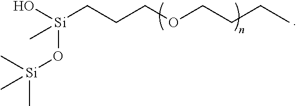

In certain embodiments, an organosilicone preparation that comprises an organosilicone compound comprising a trisiloxane head group is used in the methods and compositions provided herein. In certain embodiments, an organosilicone preparation that comprises an organosilicone compound comprising a heptamethyltrisiloxane head group is used in the methods and compositions provided herein. In certain embodiments, an organosilicone composition that comprises Compound I is used in the methods and compositions provided herein. In certain embodiments, an organosilicone composition that comprises Compound I is used in the methods and compositions provided herein. In certain embodiments of the methods and compositions provided herein, a composition that comprises a polynucleotide molecule and one or more effective organosilicone compound in the range of about 0.015 to about 2 percent by weight (wt percent) (e. g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) is used or provided.

In certain embodiments, the polynucleotide compositions that comprise an organosilicone preparation can comprise a salt such as ammonium chloride, tetrabutylphosphonium bromide, and/or ammonium sulfate. Ammonium chloride, tetrabutylphosphonium bromide, and/or ammonium sulfate can be provided in the polynucleotide composition at a concentration of about 0.5% to about 5% (w/v). An ammonium chloride, tetrabutylphosphonium bromide, and/or ammonium sulfate concentration of about 1% to about 3%, or about 2% (w/v) can also be used in the polynucleotide compositions that comprise an organosilicone preparation. In certain embodiments, the polynucleotide compositions can comprise an ammonium salt at a concentration greater or equal to 300 millimolar. In certain embodiments, the polynucleotide compositions that comprise an organosilicone preparation can comprise ammonium sulfate at concentrations from about 80 to about 1200 mM or about 150 mM to about 600 mM.

In certain embodiments, the polynucleotide compositions can also comprise a phosphate salt. Phosphate salts used in the compositions include, but are not limited to, calcium, magnesium, potassium, or sodium phosphate salts. In certain embodiments, the polynucleotide compositions can comprise a phosphate salt at a concentration of at least about 5 millimolar, at least about 10 millimolar, or at least about 20 millimolar. In certain embodiments, the polynucleotide compositions will comprise a phosphate salt in a range of about 1 mM to about 25 mM or in a range of about 5 mM to about 25 mM. In certain embodiments, the polynucleotide compositions can comprise sodium phosphate at a concentration of at least about 5 millimolar, at least about 10 millimolar, or at least about 20 millimolar. In certain embodiments, the polynucleotide compositions can comprise sodium phosphate at a concentration of about 5 millimolar, about 10 millimolar, or about 20 millimolar. In certain embodiments, the polynucleotide compositions will comprise a sodium phosphate salt in a range of about 10 mM to about 160 mM or in a range of about 20 mM to about 40 mM. In certain embodiments, the polynucleotide compositions can comprise a sodium phosphate buffer at a pH of about 6.8.

In certain embodiments, other useful transfer agents or adjuvants to transfer agents that can be used in polynucleotide compositions provided herein include surfactants and/or effective molecules contained therein. Surfactants and/or effective molecules contained therein include, but are not limited to, sodium or lithium salts of fatty acids (such as tallow or tallowamines or phospholipids) and organosilicone surfactants. In certain embodiments, the polynucleotide compositions that comprise a transfer agent are formulated with counter-ions or other molecules that are known to associate with nucleic acid molecules. Illustrative examples include, but are not limited to, tetraalkyl ammonium ions, trialkyl ammonium ions, sulfonium ions, lithium ions, and polyamines such as spermine, spermidine, or putrescine. In certain embodiments, the polynucleotide compositions are formulated with a non-polynucleotide herbicide. Non-polynucleotide herbicidal molecules include, but are not limited to, glyphosate, auxin-like benzoic acid herbicides including dicamba, chloramben and TBA, glufosinate, auxin-like herbicides including phenoxy carboxylic acid herbicide, pyridine carboxylic acid herbicide, quinoline carboxylic acid herbicide, pyrimidine carboxylic acid herbicide, and benazolin-ethyl herbicide, sulfonylureas, imidazolinones, bromoxynil, delapon, cyclohezanedione, protoporphyrionogen oxidase inhibitors, and 4-hydroxyphenyl-pyruvate-dioxygenase inhibiting herbicides.

In certain embodiments, the polynucleotides used in the compositions that are essentially identical or essentially complementary to the PMR5 target gene or transcript will comprise the predominant nucleic acid in the composition. Thus in certain embodiments, the polynucleotides that are essentially identical or essentially complementary to the target gene or transcript will comprise at least about 50%, 75%, 95%, 98%, or 100% of the nucleic acids provided in the composition by either mass or molar concentration. However, in certain embodiments, the polynucleotides that are essentially identical or essentially complementary to the target gene or transcript can comprise at least about 1% to about 50%, about 10% to about 50%, about 20% to about 50%, or about 30% to about 50% of the nucleic acids provided in the composition by either mass or molar concentration. Also provided are compositions where the polynucleotides that are essentially identical or essentially complementary to the target gene or transcript can comprise at least about 1% to 100%, about 10% to 100%, about 20% to about 100%, about 30% to about 50%, or about 50% to a 100% of the nucleic acids provided in the composition by either mass or molar concentration.

Polynucleotides comprising ssDNA, dsDNA, ssRNA, dsRNA, or RNA/DNA hybrids that are essentially identical or complementary to certain plant target genes or transcripts and that can be used in compositions containing transfer agents that include, but are not limited to, organosilicone preparations, to suppress those target genes when topically applied to plants are disclosed in co-assigned U.S. patent application Ser. No. 13/042,856 (US20110296556). Various polynucleotide herbicidal molecules, compositions comprising those polynucleotide herbicidal molecules and transfer agents that include, but are not limited to, organosilicone preparations, and methods whereby herbicidal effects are obtained by the topical application of such compositions to plants are also disclosed in co-assigned U.S. patent application Ser. No. 13/042,856, and those polynucleotide herbicidal molecules, compositions, and methods are incorporated herein by reference in their entireties. Genes encoding proteins that can provide tolerance to an herbicide and/or that are targets of a herbicide are collectively referred to herein as “herbicide target genes”. Herbicide target genes include, but are not limited to, a 5-enolpyruvylshikimate-3-phosphate synthase (EPSPS), a glyphosate oxidoreductase (GOX), a glyphosate decarboxylase, a glyphosate-N-acetyl transferase (OAT), a dicamba monooxygenase, a phosphinothricin acetyltransferase, a 2,2-dichloropropionic acid dehalogenase, an acetohydroxyacid synthase, an acetolactate synthase, a haloarylnitrilase, an acetyl-coenzyme A carboxylase (ACCase), a dihydropteroate synthase, a phytoene desaturase (PDS), a protoporphyrin IX oxygenase (PPO), a hydroxyphenylpyruvate dioxygenase (HPPD), a para-aminobenzoate synthase, a glutamine synthase, a cellulose synthase, a beta tubulin, and a serine hydroxymethyltransferase gene. The effects of applying certain compositions comprising polynucleotides that are essentially identical or complementary to certain herbicide target genes and transfer agents on plants containing the herbicide target genes was shown to be potentiated or enhanced by subsequent application of an herbicide that targets the same gene as the polynucleotide in co-assigned U.S. patent application Ser. No. 13/042,856 (US20110296556). For example, compositions comprising polynucleotides targeting the EPSPS herbicide target gene were potentiated by glyphosate in experiments disclosed in co-assigned U.S. patent application Ser. No. 13/042,856 (US20110296556).

In certain embodiments of the compositions and methods disclosed herein, the composition comprising a polynucleotide and a transfer agent can thus further comprise a second polynucleotide comprising at least 19 contiguous nucleotides that are essentially identical or essentially complementary to a transcript to a protein that confers resistance to a herbicide. In certain embodiments, the second polynucleotide does not comprise a polynucleotide that is essentially identical or essentially complementary to a transcript encoding a protein of a target plant that confers resistance to said herbicidal molecule. Thus, in an non-limiting embodiment, the second polynucleotide could be essentially identical or essentially complementary to a transcript encoding a protein that confers resistance to a herbicide in a weed (such as an EPSPS encoding transcript) but would not be essentially identical or essentially complementary to a transcript encoding a protein that confers resistance to that same herbicide in a crop plant.

In certain embodiments, the polynucleotide compositions that comprise a transfer agent can comprise glycerin. Glycerin can be provided in the composition at a concentration of about 0.1% to about 1% (w/v or v/v). A glycerin concentration of about 0.4% to about 0.6%, or about 0.5% (w/v or v/v) can also be used in the polynucleotide compositions that comprise a transfer agent.

In certain embodiments, the polynucleotide compositions that comprise a transfer agent can further comprise organic solvents. Such organic solvents include, but are not limited to, DMSO, DMF, pyridine, N-pyrrolidine, hexamethylphosphoramide, acetonitrile, dioxane, polypropylene glycol, other solvents miscible with water or that will dissolve phosphonucleotides in non-aqueous systems (such as is used in synthetic reactions).

In certain embodiments, the polynucleotide compositions that comprise a transfer agent can further comprise naturally derived or synthetic oils with or without surfactants or emulsifiers. Such oils include, but are not limited to, plant-sourced oils, crop oils (such as those listed in the 9th Compendium of Herbicide Adjuvants, publicly available on line at www.herbicide.adjuvants.com), paraffinic oils, polyol fatty acid esters, or oils with short-chain molecules modified with amides or polyamines such as polyethyleneimine or N-pyrrolidine.

In some embodiments, methods include one or more applications of the composition comprising a polynucleotide and a transfer agent or one or more effective components contained therein. In certain embodiments of the methods, one or more applications of a transfer agent or one or more effective components contained therein can precede one or more applications of the composition comprising a polynucleotide and a transfer agent. In embodiments where a transfer agent and/or one or more effective molecules contained therein is used either by itself as a pre-treatment or as part of a composition that includes a polynucleotide, embodiments of the polynucleotide molecules are double-stranded RNA oligonucleotides, single-stranded RNA oligonucleotides, double-stranded RNA polynucleotides, single-stranded RNA polynucleotides, double-stranded DNA oligonucleotides, single-stranded DNA oligonucleotides, double-stranded DNA polynucleotides, single-stranded DNA polynucleotides, chemically modified RNA or DNA oligonucleotides or polynucleotides or mixtures thereof:

Compositions and methods as described herein are useful for modulating or suppressing the expression of an endogenous PMR5 target gene or transgenic PMR5 target gene in a plant cell or plant. In certain embodiments of the methods and compositions provided herein, expression of PMR5 target genes can be suppressed completely, partially and/or transiently to result in an improvement in fungal disease resistance and/or nematode resistance. In various embodiments, a PMR5 target gene includes coding (protein-coding or translatable) sequence, non-coding (non-translatable) sequence, or both coding and non-coding sequence. In some embodiments, compositions can include polynucleotides and oligonucleotides designed to target multiple PMR5 genes, or multiple segments of one or more PMR5 genes. The target gene can include multiple consecutive segments of a target PMR5 gene, multiple non-consecutive segments of a PMR5 target gene, multiple alleles of a target gene, or multiple PMR5 target genes from one or more species. PMR5 target genes include, but are not limited to, the endogenous PMR5 plant genes of SEQ ID NO: 1, 2, 3, 4, 5, 6, 7, 8, 9, 10, or 11. PMR5 target genes include, but are not limited to, PMR5 plant genes that encode proteins that are orthologous to the proteins of SEQ ID NO:41-48, or 49. PMR5 target genes include, but are not limited to, PMR5 plant genes that encode the proteins of SEQ ID NO:41-48, or 49 or essentially homologous proteins having between about 1, 2, 3, 4, 5, 6, 7, 8, 9, or 10 amino acid substitutions, deletions, or insertions.

Target genes and plants containing those target genes can be obtained from: i) row crop plants including, but are not limited to, corn, soybean, cotton, canola, sugar beet, alfalfa, sugarcane, rice, and wheat; ii) vegetable plants including, but not limited to, tomato, potato, sweet pepper, hot pepper, melon, watermelon, cucumber, eggplant, cauliflower, broccoli, lettuce, spinach, onion, peas, carrots, sweet corn, Chinese cabbage, leek, fennel, pumpkin, squash or gourd, radish, Brussels sprouts, tomatillo, garden beans, dry beans, or okra; iii) culinary plants including, but not limited to, basil, parsley, coffee, or tea; iv) fruit plants including but not limited to apple, pear, cherry, peach, plum, apricot, banana, plantain, table grape, wine grape, citrus, avocado, mango, or berry; v) a tree grown for ornamental or commercial use, including, but not limited to, a fruit or nut tree; or, vi) an ornamental plant (e.g., an oramental flowering plant or shrub or turf grass). The methods and compositions provided herein can also be applied to plants produced by a cutting, cloning, or grafting process (i.e., a plant not grown from a seed) include fruit trees and plants that include, but are not limited to, citrus, apples, avocados, tomatoes, eggplant, cucumber, melons, watermelons, and grapes as well as various ornamental plants. Such row crop, vegetable, culinary, fruit, tree, or ornamental plants exhibiting improvements in fungal disease resistance and/or nematode resistance that result from suppressing PMR5 gene expression are provided herein. Such row crop, vegetable, culinary, fruit, tree, or ornamental plant parts or processed plant products exhibiting improvements in fungal disease resistance and/or nematode resistance that result from suppressing PMR5 gene expression are also provided herein. Such plant parts can include, but are not limited to, flowers, stems, tubers, fruit, anthers, meristems, ovules, pollen, leaves, or seeds. Such processed plant products obtained from the plant parts can include, but are not limited to, a meal, a pulp, a feed, or a food product.

A method for modulating or suppressing expression of an PMR5 gene in a plant including (a) conditioning of a plant to permeation by polynucleotides and (b) treatment of the plant with the polynucleotide molecules, wherein the polynucleotide molecules include at least one segment of 18 or more contiguous nucleotides cloned from or otherwise identified from the PMR5 target gene in either anti-sense or sense orientation, whereby the polynucleotide molecules permeate the interior of the plant and induce modulation of the target gene is provided. The conditioning and polynucleotide application can be performed separately or in a single step. When the conditioning and polynucleotide application are performed in separate steps, the conditioning can precede or can follow the polynucleotide application within minutes, hours, or days. In some embodiments more than one conditioning step or more than one polynucleotide molecule application can be performed on the same plant. In embodiments of the method, the segment can be cloned or identified from (a) coding (protein-encoding), (b) non-coding (promoter and other gene related molecules), or (c) both coding and non-coding parts of the PMR5 target gene. Non-coding parts include DNA, such as promoter regions or the RNA transcribed by the DNA that provide RNA regulatory molecules, including but not limited to: introns, 5′ or 3′ untranslated regions, and microRNAs (miRNA), trans-acting siRNAs, natural anti-sense siRNAs, and other small RNAs with regulatory function or RNAs having structural or enzymatic function including but not limited to: ribozymes, ribosomal RNAs, t-RNAs, aptamers, and riboswitches. In certain embodiments where the polynucleotide used in the composition comprises a promoter sequence essentially identical to, or essentially complementary to, at least 18 contiguous nucleotides of the promoter of the endogenous target gene, the promoter sequence of the polynucleotide is not operably linked to another sequence that is transcribed from the promoter sequence.

Compositions comprising a polynucleotide and a transfer agent provided herein can be topically applied to a plant or plant part by any convenient method, e.g., spraying or coating with a powder, or with a liquid composition comprising any of an emulsion, suspension, or solution. Such topically applied sprays or coatings can be of either all or of any a portion of the surface of the plant or plant part. Similarly, compositions that comprise a transfer agent or other pre-treatment can in certain embodiments be applied to the plant or plant part by any convenient method, e.g., spraying or wiping a solution, emulsion, or suspension. Compositions comprising a polynucleotide and a transfer agent provided herein can be topically applied to plant parts that include, but are not limited to, flowers, stems, tubers, meristems, ovules, fruit, anthers, pollen, leaves, or seeds.

Application of compositions comprising a polynucleotide and a transfer agent to seeds is specifically provided herein. Seeds can be contacted with such compositions by spraying, misting, immersion, and the like.

In certain embodiments, application of compositions comprising a polynucleotide and a transfer agent to plants, plant parts, or seeds in particular can provide for an improvement in fungal disease resistance and/or nematode resistance in progeny plants, plant parts, or seeds derived from those treated plants, plant parts, or seeds. In certain embodiments, progeny plants, plant parts, or seeds derived from those treated plants, plant parts, or seeds will exhibit an improvement in fungal disease resistance and/or nematode resistance that result from suppressing expression of a PMR5 gene. In certain embodiments, the methods and compositions provided herein can provide for an improvement in fungal disease resistance and/or nematode resistance in progeny plants or seeds as a result of epigenetically inherited suppression of PMR5 expression. In certain embodiments, such progeny plants exhibit an improvement in fungal disease resistance and/or nematode resistance from epigenetically inherited suppression of PMR5 gene expression that is not caused by a transgene where the polynucleotide is operably linked to a promoter, a viral vector, or a copy of the polynucleotide that is integrated into a non-native location in the chromosomal DNA of the plant. Without seeking to be limited by theory, progeny plants or seeds derived from those treated plants, plant parts, or seeds can exhibit an improvement in an improvement in fungal disease resistance and/or nematode resistance through an epigenetic mechanism that provides for propagation of an epigenetic condition where suppression of PMR5 gene expression occurs in the progeny plants, plant parts, or plant seeds. In certain embodiments, progeny plants or seeds exhibiting an improvement in fungal disease resistance and/or nematode resistance as a result of epigenetically inherited suppression of PMR5 gene expression can also exhibit increased methylation, and in particular, increased methylation of cytosine residues, in the endogenous PMR5 gene of the plant. Plant parts, including seeds, of the progeny plants that exhibit an improvement in an improvement in fungal disease resistance and/or nematode resistance as a result of epigenetically inherited suppression of PMR5 gene expression, can also in certain embodiments exhibit increased methylation, and in particular, increased methylation of cytosine residues, in the endogenous PMR5 gene. In certain embodiments, DNA methylation levels in DNA encoding the endogenous PMR5 gene can be compared in plants that exhibit an improvement in fungal disease resistance and/or nematode resistance and control plants that do not exhibit an improvement in fungal disease resistance and/or nematode resistance to correlate the presence of the an improvement in fungal disease resistance and/or nematode resistance to epigenetically inherited suppression of PMR5 gene expression and to identify plants that comprise the epigentically inherited improvement in fungal disease resistance and/or nematode resistance.

Various methods of spraying compositions on plants or plant parts can be used to topically apply to a plant surface a composition comprising a polynucleotide that comprises a transfer agent. In the field, a composition can be applied with a boom that extends over the crops and delivers the composition to the surface of the plants or with a boomless sprayer that distributes a composition across a wide area. Agricultural sprayers adapted for directional, broadcast, or banded spraying can also be used in certain embodiments. Sprayers adapted for spraying particular parts of plants including, but not limited to, leaves, the undersides of leaves, flowers, stems, male reproductive organs such as tassels, meristems, pollen, ovules, and the like can also be used. Compositions can also be delivered aerially, such as by a crop dusting airplane. In certain embodiments, the spray can be delivered with a pressurized backpack sprayer calibrated to deliver the appropriate rate of the composition. In certain embodiments, such a backpack sprayer is a carbon dioxide pressurized sprayer with a 11015 flat fan or equivalent spray nozzle with a customized single nozzle assembly (to minimize waste) at a spray pressure of about 0.25 MPa and/or any single nozzle sprayer providing an effective spray swath of 60 cm above the canopy of 3 to 12 inch tall growing plants can be used. Plants in a greenhouse or growth chamber can be treated using a track sprayer or laboratory sprayer with a 11001XR or equivalent spray nozzle to deliver the sample solution at a determined rate. An non-limiting rate is about 140 L/ha at about 0.25 MPa pressure.

In certain embodiments, it is also contemplated that a plant part can be sprayed with the composition comprising a polynucleotide that comprises a transfer agent. Such plant parts can be sprayed either pre- or post-harvest to provide for an improvement in fungal disease resistance and/or nematode resistance in the plant part that results from suppression of PMR5 gene expression. Compositions can be topically applied to plant parts attached to a plant by a spray as previously described. Compositions can be topically applied to plant parts that are detached from a plant by a spray as previously described or by an alternative method. Alternative methods for applying compositions to detached parts include, but are not limited to, passing the plant parts through a spray by a conveyor belt or trough, or immersing the plant parts in the composition.

Compositions comprising polynucleotides and transfer agents can be applied to plants or plant parts at one or more developmental stages as desired and/or as needed. Application of compositions to pre-germination seeds and/or to post-germination seedlings is provided in certain embodiments. Seeds can be treated with polynucleotide compositions provided herein by methods including, but not limited to, spraying, immersion, or any process that provides for coating, imbibition, and/or uptake of the polynucleotide composition by the seed. Seeds can be treated with polynucleotide compositions using seed batch treatment systems or continuous flow treatment systems. Seed coating systems are at least described in U.S. Pat. Nos. 6,582,516, 5,891,246, 4,079,696, and 4,023,525. Seed treatment can also be effected in laboratory or commercial scale treatment equipment such as a tumbler, a mixer, or a pan granulator. A polynucleotide composition used to treat seeds can contain one or more other desirable components including, but not limited to liquid diluents, binders to serve as a matrix for the polynucleotide, fillers for protecting the seeds during stress conditions, and plasticizers to improve flexibility, adhesion and/or spreadability of the coating. In addition, for oily polynucleotide compositions containing little or no filler, drying agents such as calcium carbonate, kaolin or bentonite clay, perlite, diatomaceous earth or any other adsorbent material can be added. Use of such components in seed treatments is described in U.S. Pat. No. 5,876,739. Additional ingredients can be incorporated into the polynucleotide compositions used in seed treatments. Such ingredients include but are not limited to: conventional sticking agents, dispersing agents such as methylcellulose (Methocel A15LV or Methocel A15C, for example, serve as combined dispersant/sticking agents for use in seed treatments), polyvinyl alcohol (e.g., Elvanol 51-05), lecithin (e.g., Yelkinol P), polymeric dispersants (e.g., polyvinylpyrrolidone/vinyl acetate PVPNA S-630), thickeners (e.g., clay thickeners such as Van Gel B to improve viscosity and reduce settling of particle suspensions), emulsion stabilizers, surfactants, antifreeze compounds (e.g., urea), dyes, colorants, and the like that can be combined with compositions comprising a polynucleotide and a transfer agent. Further ingredients used in compositions that can be applied to seeds can be found in McCutcheon's, vol. 1, “Emulsifiers and Detergents,” MC Publishing Company, Glen Rock, N.J., U.S.A., 1996 and in McCutcheon's, vol. 2, “Functional Materials,” MC Publishing Company, Glen Rock, N.J., U.S.A., 1996. Methods of applying compositions to seeds and pesticidal compositions that can be used to treat seeds are described in U.S. Patent Application publication 20080092256, which is incorporated herein by reference in its entirety.

Application of the compositions in early, mid-, and late vegetative stages of plant development is provided in certain embodiments. Application of the compositions in early, mid- and late reproductive stages is also provided in certain embodiments. Application of the compositions to plant parts at different stages of maturation is also provided.

The following examples are included to demonstrate examples of certain embodiments. Those of skill in the art should, in light of the present disclosure, appreciate that many changes can be made in the specific embodiments that are disclosed and still obtain a like or similar result without departing from the spirit and scope of the invention.

### EXAMPLES

### Example 1. Polynucleotides Related to the PMR5 Target Gene Sequences

The target PMR5 genes and/or transcripts are provided in Table 2 and the sequence listing. Such genes, and protein sequences encoded by the PMR5 genes (Table 3), can be used to identify orthologous PMR5 genes and transcripts from other plants not provided herewith. Such orthologous genes and their transcripts can then serve as targets of polynucleotides provided herein or as a source of polynucleotides that are specifically designed to target the orthologous genes or transcripts.

The target PMR5 polynucleotide molecule at least occurs in the genome of plants provided in Table 2. The PMR5 genes provided in Table 3, or their corresponding transcripts, can be used as targets of polynucleotide compositions comprising a polynucleotide that of at least 18 contiguous nucleotides that are essentially identical or essentially complementary to those genes or transcripts. The proteins and genes respectively provided in Tables 2 and 3, or sequences contained within those proteins or genes can also be used to obtain orthologous PMR5 genes from plants not listed in Tables 2 and 3. Such orthologous genes and their transcripts can then serve as targets of polynucleotides provided herein or as a source of polynucleotides that are specifically designed to target the orthologous genes or transcripts.

TABLE 2
Target PMR5 gene sequences

| | | |
|---|---|---|
| SEQ ID NO: 1 | Lactuca sativa | Contains PMR5-like gene |
| | (lettuce) | coding region from lettuce |
| SEQ ID NO: 2 | Solanum lycopersicum | Contains PMR5-like gene |
| | (tomato) | coding region from tomato |
| SEQ ID NO: 3 | Cucumis sativus | Contains PMR5-like gene |
| | (cucumber) | coding region from cucumber |
| SEQ ID NO: 4 | Hordeum vulgare | Contains PMR5-like gene |
| | (barley) | coding region from barley |
| SEQ ID NO: 5 | Triticum aestivum | Contains PMR5-like gene |
| | (wheat) | coding region from wheat |
| SEQ ID NO: 6 | Cucumis sp | Contains PMR5-like gene |
| | (cucumber) | coding region from cucumber |
| SEQ ID NO: 7 | Zea mays | Contains PMR5-like gene |
| | (corn) | coding region from corn |
| SEQ ID NO: 8 | Glycine max | Contains PMR5-like gene |
| | (soybean) | coding region from soybean |
| SEQ ID NO: 9 | Oryza sativa | Contains PMR5-like gene |
| | (rice) | coding region from rice |
| SEQ ID NO: 10 | Solanum lycopersicum | Contains PMR5-like |
| | (tomato) | promoter and 5' |
| | | untranslated region from |
| | | tomato PMR5-like gene |
| SEQ ID NO: 11 | Glycine max | Contains PMR5-like |
| | (soybean) | promoter and 5' |
| | | untranslated region from |
| | | soybean PMR5-like gene |

TABLE 3
Target gene encoded protein sequences

| SEQ ID NO: | Species | Description |
|---|---|---|
| SEQ ID NO: 41 | Lactuca sativa | PMR5-like protein encoded |
| | | by SEQ ID NO: 41 |
| SEQ ID NO: 42 | Solanum lycopersicum | PMR5-like protein encoded |
| | | by SEQ ID NO: 42 |
| SEQ ID NO: 43 | Cucumis sativus | PMR5-like protein encoded |
| | | by SEQ ID NO: 43 |
| SEQ ID NO: 44 | Hordeum vulgare | PMR5-like protein encoded |
| | | by SEQ ID NO: 44 |
| SEQ ID NO: 45 | Triticum aestivum | PMR5-like protein encoded |
| | | by SEQ ID NO: 45 |
| SEQ ID NO: 46 | Cucumis sp | PMR5-like protein encoded |
| | | by SEQ ID NO: 46 |
| SEQ ID NO: 47 | Zea mays | PMR5-like protein encoded |
| | | by SEQ ID NO: 47 |
| SEQ ID NO: 48 | Glycine max | PMR5-like protein encoded |
| | | by SEQ ID NO: 48 |
| SEQ ID NO: 49 | Oryza sativa | PMR5-like protein encoded |
| | | by SEQ ID NO: 49 |

The sequence listing contains the target PMR5 DNA sequences from the indicated plant species of Table 2. For each gene having a DNA sequence provided in the sequence listing and listed in Table 2, polynucleotides such as single stranded or double stranded DNA or RNA fragments in sense and/or antisense orientation will be mixed with an organosilicone preparation. These compositions will be topically applied to plants to effect expression of the target genes in the specified plant to obtain the plants that exhibit disease resistance. In particular, plants that are resistant to powdery mildew, downy mildew, and/or rust infection and/or nematodes will be obtained through the application of such compositions.

### Example 2. Polynucleotides that can be Used to Reduce PMR5 Expression in Various Plants

An set of polynucleotides that can be used to reduce expression of PMR5 genes in various plants is provided herewith as SEQ ID NOS: 12-19, 21-37, or 38. The SEQ ID NOS: 12-19, 21-37, or 38 describe ssDNA oligonucleotides and sense/antisense double stranded RNA targeted to the coding regions of PMR5 sequences from soybean and barley that are useful for downregulating PMR5 expression using methods described here. Other regions of PMR5 genes can also be targeted to modify expression including the use of antisense DNA oligonucleotides against coding regions and/or targeting promoter regions using sense/antisense dsRNA, sense or antisense ssDNA as well as sense/antisense double stranded DNA. For example, a polynucleotide that comprises at least 18 contiguous nucleotides that are essentially identical or essentially complementary to SEQ ID NO: 1, 2, 3, 4, 5, 6, 7, 8, 9, 10, or 11 can be used to downregulate expression of those PMR5 genes.

### Example 3. Topical Oligonucleotide Application and Powdery Mildew Testing Methods

Barley seeds (Perry variety) are planted about ¼″ into soil in 2 inch pots in the growth chamber and grown at 25° C. with a 16 hr light cycle in 50% humidity. Before polynucleotide application the plants are randomized. Application of polynucleotides (either ssDNA oligos and/or dsRNA) is performed by pipet application where 5 μL of solution containing nucleotides is applied to both sides of the first leaf. The nucleotide solution applied consists of ˜3-15 nmol of each ssDNA oligonucleotide or ˜0.5-1 nmol dsRNA, 0.1-0.3% Silwet L-77, 5 mM NaPO₄, and 1% AMS in Gibco ultra-pure water. Examples of polynucleotides include polynucleotides that comprise at least 18 contiguous nucleotides that are essentially identical or complementary to SEQ ID NO:4. Examples of polynucleotides also include polynucleotides of SEQ ID NO:21-37, and 38. Two days post treatment seedlings are infected with barley powdery mildew (Blumeria graminis f. sp. hordei). The growth chamber settings for the infection are as follows: 23° C., with a 12 hr light cycle in 70% humidity. At seven days post infection disease severity is scored for the percentage of leaf area covered with powdery mildew.

Data is analyzed using ANOVA Single Factor Analysis (α=0.1). The ½ LSD is calculated and custom error bars created for the bar graphs. Percent disease reduction is compared to formulation blank and nucleic acid control.

Cucumber seeds are planted in a 3-inch square pot and thinned to one plant per pot after emergence. When the first true leaf is fully expanded and the second leaf is opening, a polynucleotide solution such as ssDNA and/or dsRNA oligos directed to the promoter and/or targeting the coding region of a target gene of interest is applied to the first true leaf or the cotyledons. Examples of polynucleotides include polynucleotides that comprise at least 18 contiguous nucleotides that are essentially identical or complementary to SEQ ID NO:3 or 6. The nucleotide solution applied consists of 6-20 nm of each ssDNA oligonucleotide or 0.5-4 nm dsRNA, 0.1 to 0.3% L77 Silwet, 50 mM NaPO₄ in a final volume of 40 microliters of water. Two days later the entire cucumber plant is inoculated with a shower of dry spores of cucumber powdery mildew (Sphaerotheca fuliginea) shaken off diseased plants. Disease severity will be evaluated on the treated leaf and succeeding leaves 10 days later and at subsequent intervals.

Tomato seeds are planted in a 3-inch square pot and thinned to one plant per pot after emergence. Two weeks old tomato seedlings are treated with 6-20 nm of each ssDNA oligonucleotide or 0.5-4 nm dsRNA, 0.2-0.5% L77 Silwet, 50 mM NaPO₄, 1% ammonium sulfate in a final volume of 30 microliters of water. Examples of polynucleotides include polynucleotides that comprise at least 18 contiguous nucleotides that are essentially identical or complementary to SEQ ID NO:2 or 10. Two to 4 days post spraying plants are inoculated with dry spores of tomato powdery mildew (Oidium neolycopersici) and 13 days post infection, disease development is scored for the percentage of leaf area covered with powdery mildew.

### Example 4. Protection of Barley from Powdery Mildew by Topical Application of Pooled ssDNA Oligonucleotides

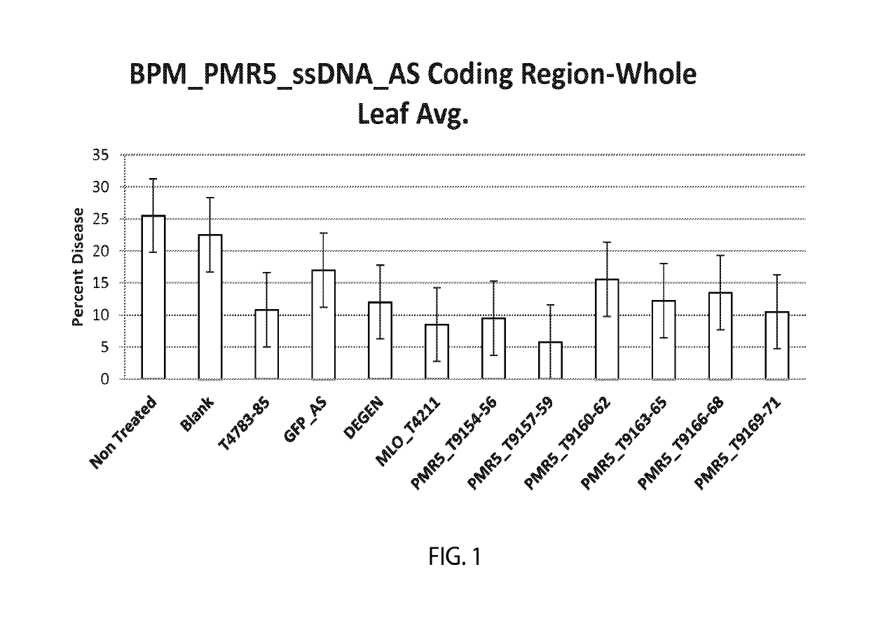

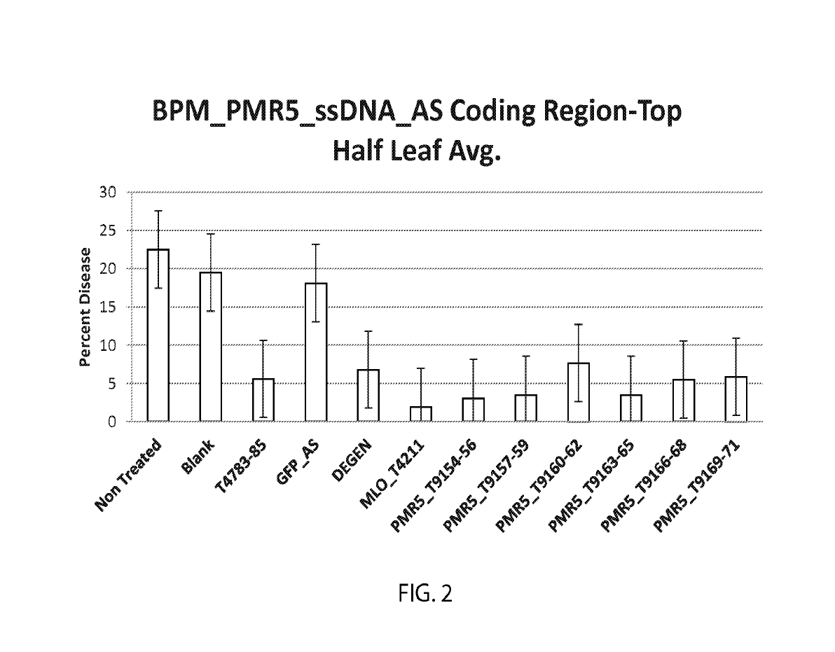

Barley seeds are planted in 2 inch pots in the greenhouse essentially as described in Example 3. Six days later, barley seedlings were treated with the indicated oligos or a control formulation according to the method outlined in Table 4, where 4 μl of the indicated solution is applied to both sides of a leaf. Treatment 1-12 methods with the indicated ssDNA oligonucleotide(s) are described in Table 5. The description of the ssDNA oligonucleotides used is provided in Table 6. About 2 days post treatment, the seedlings were infected with spores of barley powdery mildew (Blumeria graminis f. sp. hordei) and 7 days post infection, disease development was scored for the percentage of leaf area covered with powdery mildew. Results of this analysis are shown in FIGS. 1, 2 and corresponding Tables 7 and 8, respectively. ANOVA statistical calculations for the Table 7 are shown in Table 9 and the corresponding graph with LSD bar is shown in FIG. 1. ANOVA statistical calculations for the Table 8 are shown in Table 10 and the corresponding graph with LSD bar is shown in FIG. 2.

TABLE 4
Formulation

| Component | Final Concentration | |
|---|---|---|
| PMR5 or Control Trigger | 1.52 | nmol |
| Oligonucleotide | | |
| NaPO4 | 5 | mM |

| | |
|---|---|
| AMS (Ammonium | 1% |
| Sulfate) | |
| Silwet | 0.25% |

TABLE 5
Treatments

| | Trigger | | | target | | |
|---|---|---|---|---|---|---|
| Trmnt | Type | Nucleotide ID# | Nucleotide Conc. | gene | region | reps |
| 1 | X | Non Treated | X | X | X | 5 |
| 2 | X | Blank | X | X | X | 5 |
| 3 | ssDNA_AS | T4783-85¹ | .51 nmol/oligo × 3 | X | X | 5 |
| | | | oligos | | | |
| 4 | ssDNA_AS | GFP_AS² (SEQ | 1.52 nmol/oligo | X | X | 5 |
| | | ID NO: 20) | | | | |
| 5 | ssDNA_AS | DEGEN³ | 1.52 nmol/oligo | X | X | 5 |
| 6 | ssDNA_AS | T4211 (SEQ ID | 1.52 nmol/oligo | MLO⁴ | CDS | 5 |
| | | NO: 39) | | | | |
| 7 | ssDNA_AS | T9154-56 | .51 nmol/oligo × 3 | PMR5 | CDS | 5 |
| | | (SEQ ID NO: 21, | oligos | | | |
| | | 22, 23) | | | | |
| 8 | ssDNA_AS | T9157-59 (SEQ | .51 nmol/oligo × 3 | PMR5 | CDS | 5 |
| | | ID NO: 24, 25, | oligos | | | |
| | | 26) | | | | |
| 9 | ssDNA_AS | T9160-62 (SEQ | .51 nmol/oligo × 3 | PMR5 | CDS | 5 |
| | | ID NO: 27, 28, | oligos | | | |
| | | 29) | | | | |
| 10 | ssDNA_AS | T9163-65 (SEQ | .51 nmol/oligo × 3 | PMR5 | CDS | 5 |
| | | ID NO: 30, 31, | oligos | | | |
| | | 32) | | | | |
| 11 | ssDNA_AS | T9166-68 (SEQ | .51 nmol/oligo × 3 | PMR5 | CDS | 5 |
| | | ID NO: 33, 34, | oligos | | | |
| | | 35) | | | | |
| 12 | ssDNA_AS | T9169-71 (SEQ | .51 nmol/oligo × 3 | PMR5 | CDS | 5 |
| | | ID NO: 33, 34, | oligos | | | |
| | | 35 | | | | |

¹T4783-85 are randomly generated oligos that do not match cucumber, cotton, tomato, melon, lettuce, barley, soybean, maize genomes at >=20 bp with 100% identity (negative control).
²GFP_AS is an oligonucleotide (SEQ ID NO: 20) directed against the Aqueoria Green Fluorescent Protein (negative control).
³DEGEN are a mixture of degenerate oligonucleotides (negative control).
⁴MLO is an positive control oligonucleotide (SEQ ID NO: 39) that targets a barley (Mildew Resistance Locus O) gene(s).

TABLE 6
Oligonucleotides Used

| Oligo | | SEQ ID | |
|---|---|---|---|
| name | Sequence | NO: | Length |
| T4782 | ATGGGGGCTCCCGTTAATCCGAAGA | 40 | 25 |
| T4783 | AGCGCCGGTAGCGAGCATACGTATG | 50 | 25 |
| T4784 | ACGACTCTGCTTATTATACTCGGTC | 51 | 25 |
| T4784 | GACATATTAGGGGCGACGTCTCCAA | 52 | 25 |
| T9154 | GCGGAGCCGTCGACATCGCGGACCC | 21 | 25 |
| T9155 | CACTTGTACCCGGTGTAGCCCTCCG | 22 | 25 |
| T9156 | AGTTGAACTCCGCGTCGATGACCGG | 23 | 25 |
| T9157 | GGAGTCCGGGCGGCCATAGAGCTGG | 24 | 25 |
| T9158 | CGGCTTCCAGCGGTACCGGAGGTAG | 25 | 25 |
| T9159 | GTCAAACCTGGGTAGCTCGCAGCTG | 26 | 25 |
| T9160 | CTTCATCCGCGTCAAAAAGTCGGCG | 27 | 25 |
| T9161 | TCCCCCACGAACATCACCGTCTTCC | 28 | 25 |
| T9162 | CGACTCCCACTGGTTACGGCCCAGC | 29 | 25 |
| T9163 | GCGCGGCGGCGTGCAGCAGGCAGAC | 30 | 25 |
| T9164 | GGTCGGCGGAGACGAGCTGCGACGG | 31 | 25 |
| T9165 | TCCCCTGCACCACGTCGATGTCCAC | 32 | 25 |
| T9166 | GAGGTCCACCCAGTTGGCCCATGTG | 33 | 25 |
| T9167 | TTATTGGGCCCAGCCAACGGGACCG | 34 | 25 |
| T9168 | GCTCCTGGCCCATCGGCTGCGACGT | 35 | 25 |
| T9169 | CCCTGCAGCACCGTTTTCGTCACCT | 36 | 25 |
| T9170 | GAGCAAACGGACCGGGCTTTTCATC | 37 | 25 |
| T9171 | CGCAGCGCCGACAGCGCCGTGATGT | 38 | 25 |

TABLE 7
Results of Whole Leaf Assay

| SUMMARY: Whole Leaf | | | | |
|---|---|---|---|---|
| | | | Average | |
| | | | Percent Leaf | |
| | | | Area | |
| Groups | Count | Sum | Diseased | Variance |
| Non Treated | 10 | 255 | 25.5 | 213.6111 |
| Blank | 10 | 225 | 22.5 | 256.9444 |
| T4783-85 (Random | 10 | 108 | 10.8 | 106.8444 |
| Oligonucleotide | | | | |
| negative control) | | | | |
| GFP_AS | 10 | 170 | 17 | 173.3333 |
| DEGEN | 10 | 120 | 12 | 51.11111 |
| T4211 | 10 | 85 | 8.5 | 39.16667 |
| T9154-56 | 10 | 95 | 9.5 | 35.83333 |
| T9157-59 | 10 | 58 | 5.8 | 15.73333 |
| T9160-62 | 10 | 156 | 15.6 | 204.7111 |
| T9163-65 | 10 | 122 | 12.2 | 223.7333 |
| T166-68 | 10 | 135 | 13.5 | 66.94444 |
| T9169-71 | 10 | 105 | 10.5 | 63.61111 |

TABLE 8
Results of Top-half Leaf Analysis

| SUMMARY: top half of leaf | | | | |
|---|---|---|---|---|
| | | | Average | |
| | | | Percent Leaf | |
| | | | Area | |
| Groups | Count | Sum | Diseased | Variance |
| Non Treated | 10 | 225 | 22.5 | 390.2778 |
| Blank | 10 | 195 | 19.5 | 180.2778 |
| T4783-85 | 10 | 56 | 5.6 | 60.26667 |
| GFP_AS | 10 | 181 | 18.1 | 219.4333 |
| DEGEN | 10 | 68 | 6.8 | 51.73333 |
| T4211 | 10 | 19 | 1.9 | 8.1 |
| T9154-56 | 10 | 31 | 3.1 | 14.98889 |
| T9157-59 | 10 | 35 | 3.5 | 9.166667 |
| T9160-62 | 10 | 77 | 7.7 | 48.23333 |
| T9163-65 | 10 | 35 | 3.5 | 14.72222 |
| T9166-68 | 10 | 55 | 5.5 | 55.83333 |
| T9169-71 | 10 | 59 | 5.9 | 53.43333 |

TABLE 9
ANOVA Analysis of Table 7 Data (single factor)

| ANOVA | | | | | | |
|---|---|---|---|---|---|---|
| Source of Variation | SS | df | MS | F | P-value | F crit |
| Between Groups | 3620.167 | 11 | 329.1060606 | 2.720676 | 0.003881 | 1.631129 |
| Within Groups | 13064.2 | 108 | 120.9648148 | | | |
| Total | 16684.37 | 119 | | | | |
| | | Std of | 48.38592593 | 6.955999 | | |
| | | dif | | | | |
| | | DF | 1.6606 | | | |
| | | LSD | 11.5511324 | | | |
| | | LSD/2 | 5.775566199 | | | |

TABLE 10
ANOVA Analysis of Table 8 Data (single factor)

| ANOVA | | | | | | |
|---|---|---|---|---|---|---|
| Source of Variation | SS | df | MS | F | P-value | F crit |
| Between Groups | 5593.667 | 11 | 508.5151515 | 5.515016 | 6.1133E−07 | 1.631129 |
| Within Groups | 9958.2 | 108 | 92.20555556 | | | |
| Total | 15551.87 | 119 | | | | |
| | | Std of | 36.88222222 | 6.073074 | | |
| | | dif | | | | |
| | | DF | 1.6606 | | | |
| | | LSD | 10.08494592 | | | |
| | | LSD/2 | 5.04247296 | | | |

FIGS. 1 and 2 show that the percentage disease area was decreased numerically in plants treated with Silwet formulations containing certain barley PMR5 antisense DNA oligonucleotides relative to the Silwet formulation alone (Blank), the Silwet formulation combined with degenerate oligo mixture, or the Silwet formulation combined with a control GFP (Green Fluorescent Protein) oligonucleotide (SEQ ID NO:20).

FIG. 2 shows that the percent leaf disease area was decreased by a statistically significant level in plants treated with Silwet formulations containing certain barley PMR5 antisense DNA oligonucleotides relative to the Silwet formulation alone (Blank) and the Silwet formulation combined with a control GFP (Green Fluorescent Protein) oligonucleotide (SEQ ID NO:20).

### Example 5. Protection of Barley from Powdery Mildew by Topical Application of Single PMR5 Oligonucleotides

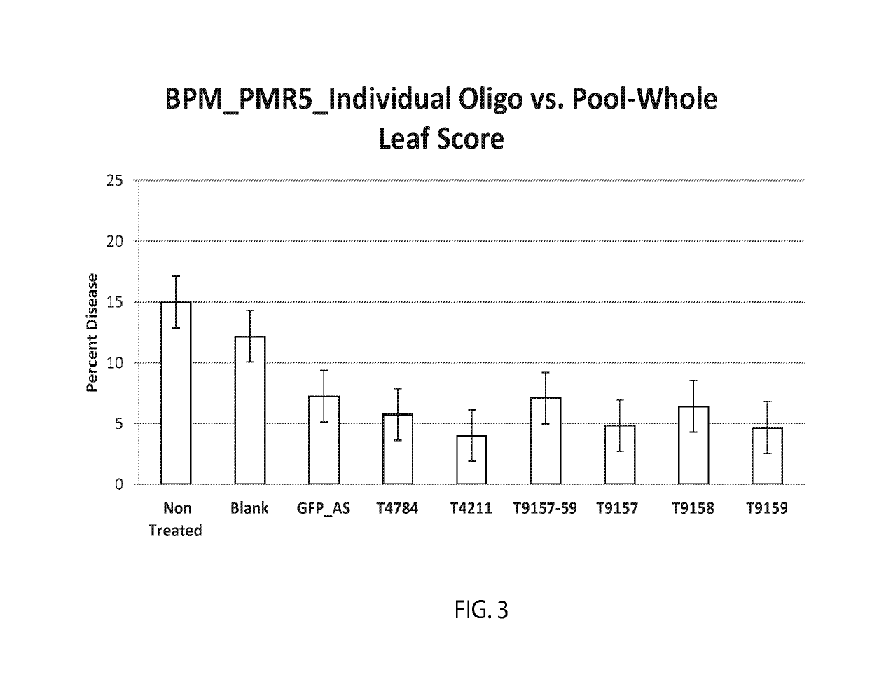

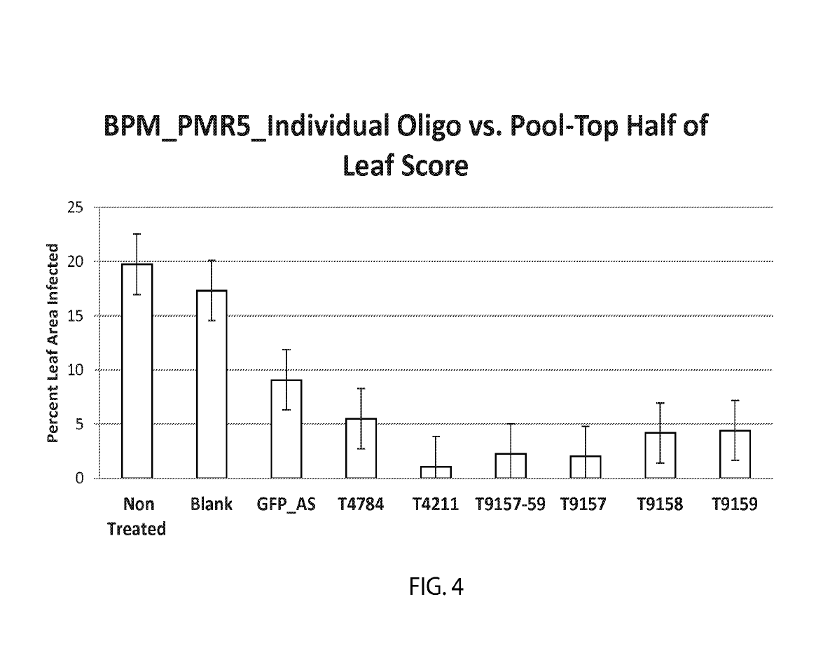

Barley seeds are planted in 2 inch pots in the greenhouse essentially as described in Example 3. Six days later, barley seedlings were treated with the indicated oligos or a control formulation according to the method outlined in Table 11, where 4 μl of the indicated solution is applied to both sides of a leaf. Treatment 1-9 methods with the indicated ssDNA oligonucleotide(s) are described in Table 12. The description of the ssDNA oligonucleotides used is provided in Table 13. About 2 days post oligonucleotide treatment, the seedlings were infected with spores of barley powdery mildew (Blumeria graminis f. sp. hordei) and 7 days post infection, disease development was scored for the percentage of leaf area covered with powdery mildew. Results of this analysis are shown in FIGS. 3, 4 and corresponding Tables 14 and 15, respectively. ANOVA statistical calculations for the Table 14 results are shown in Table 16 and the corresponding graph with LSD bar is shown in FIG. 3. ANOVA statistical calculations for the Table 15 are shown in Table 17 and the corresponding graph with LSD bar is shown in FIG. 4.

TABLE 11
Formulations

| Component | Final Concentration | |
|---|---|---|
| PMR5 or Control Trigger | 1.14 | nmol |
| Oligonucleotide | | |
| NaPO₄ | 5 | mM |

| | |
|---|---|
| AMS (Ammonium | 1% |
| Sulfate) | |
| Silwet | 0.25% |

TABLE 12
Treatments

| | | | target | | |
|---|---|---|---|---|---|
| Trtmnt | Nucleotide ID# | Nucelotide Conc. | gene | region | reps |
| 1 | Non Treated | X | X | X | 6 |
| 2 | Blank | X | X | X | 6 |
| 3 | GFP_AS | 1.14 nmol/oligo | X | X | 6 |
| | (SEQ ID NO: 20) | | | | |
| 4 | T4784 | 1.14 nmol/oligo | X | X | 6 |
| | (SEQ ID NO: 51) | | | | |
| 5 | T4211 | 1.14 nmol/oligo | MLO | CDS | 6 |
| | (SEQ ID NO: 39) | | | | |
| 6 | T9157-59 | .38 nmol/oligo X | PMR5 | CDS | 6 |
| | oligonucleotide | 3 oligos | | | |
| | pool (SEQ ID | | | | |
| | NO: 24, 25, 26) | | | | |
| 7 | T9157 | 1.14 nmol/oligo | PMR5 | CDS | 6 |
| 8 | T9158 | 1.14 nmol/oligo | PMR5 | CDS | 6 |
| 9 | T9159 | 1.14 nmol/oligo | PMR5 | CDS | 6 |

TABLE 13
Oligonucleotides Used

| Oligo | | SEQ ID | |
|---|---|---|---|
| name | Sequence | NO: | Length |
| T4784 | ACGACTCTGCTTATTATACTCGGTC | 51 | 25 |
| T9157 | GGAGTCCGGGCGGCCATAGAGCTGG | 24 | 25 |
| T9158 | CGGCTTCCAGCGGTACCGGAGGTAG | 25 | 25 |
| T9159 | GTCAAACCTGGGTAGCTCGCAGCTG | 26 | 25 |

TABLE 14
Results of Whole Leaf Assay

| | | | Average | |
|---|---|---|---|---|
| | | | Percent | |
| Treatment | Count | Sum | Disease | Variance |
| Non Treated | 6 | 90 | 15 | 7.5 |
| Blank | 6 | 73 | 12.2 | 49.66667 |
| GFP_AS | 6 | 43.5 | 7.25 | 9.275 |
| T4784 | 6 | 34.5 | 5.75 | 11.775 |
| T4211 | 6 | 24 | 4 | 30 |
| T9157-59 | 6 | 42.5 | 7.08 | 35.14167 |
| T9157 | 6 | 29 | 4.83 | 2.866667 |
| T9158 | 6 | 38.5 | 6.42 | 5.941667 |
| T9159 | 6 | 28 | 4.67 | 19.46667 |

TABLE 15
Results of Top-half Leaf Analysis
SUMMARY

| | | | Average Percent | |
|---|---|---|---|---|
| Groups | Count | Sum | Disease | Variance |
| Non Treated | 6 | 118.5 | 19.75 | 83.275 |
| Blank | 6 | 104 | 17.3 | 66.86667 |
| GFP_AS | 6 | 54.5 | 9.08 | 111.5417 |
| T4784 | 6 | 33 | 5.5 | 15 |
| T4211 | 6 | 6.5 | 1.08 | 0.941667 |
| T9157-59 | 6 | 13.5 | 2.25 | 3.475 |
| T9157 | 6 | 12 | 2 | 1.4 |
| T9158 | 6 | 25 | 4.17 | 3.266667 |
| T9159 | 6 | 26.5 | 4.42 | 8.241667 |

TABLE 16
ANOVA Analysis of Table 14 Data (single factor)

| Source of Variation | SS | df | MS | F | P-value | F crit |
|---|---|---|---|---|---|---|
| Between Groups | 659.2593 | 8 | 82.40741 | 4.321227 | 0.000641 | 1.810719 |
| Within Groups | 858.1667 | 45 | 19.07037 | | | |
| Total | 1517.426 | 53 | | | | |
| | Std of dif | 6.35679 | 2.521268 | | | |
| | DF | 1.6808 | | | | |
| | LSD | 4.237747 | | | | |
| | LSD/2 | 2.118873 | | | | |

TABLE 17
ANOVA Analysis of Table 15 Data (single factor)

| Source of Variation | SS | df | MS | F | P-value | F crit |
|---|---|---|---|---|---|---|
| Between Groups | 2234.759259 | 8 | 279.3449 | 8.551132 | 5.42746E−07 | 1.810719 |
| Within Groups | 1470.041667 | 45 | 32.66759 | | | |
| Total | 3704.800926 | 53 | | | | |
| | | Std of | 10.8892 | 3.299878 | | |
| | | dif | | | | |
| | | DF | 1.6808 | | | |
| | | LSD | 5.546436 | | | |
| | | LSD/2 | 2.773218 | | | |

FIGS. 3 and 4 show that the percentage disease area was decreased numerically in plants treated with Silwet formulations containing certain barley PMR5 antisense DNA oligonucleotides relative to both the Silwet formulation alone (Blank), or the Silwet formulation combined with a control GFP (Green Fluorescent Protein) oligonucleotide (SEQ ID NO:20).

FIG. 4 shows that the percentage disease area was decreased by a statistically significant level in plants treated with Silwet formulations containing certain barley PMR5 antisense DNA oligonucleotides relative to both the Silwet formulation alone (Blank) and the Silwet formulation combined with a control GFP (Green Fluorescent Protein) oligonucleotide (SEQ ID NO:20).

### Example 6. Topical Oligonucleotide Application and Nematode Testing Methods Application of Oligonucleotides to Leaves for Nematode Control

Ten day old cucumber plants grown in sand are spotted with nucleotides, either ssDNA and/or dsRNA oligos directed to the promoter and/or targeting the coding region of a target gene of interest. The nucleotide solution applied consists of 6-20 nm of each ssDNA oligonucleotide or 0.5-1 nm dsRNA, 0.1% L77 Silwet, 50 mM NaPO4 in a final volume of 40 uL water. Two cotyledon or leaves are spotted with 20 uL of the nucleotide solution for a total of 40 uL per plant. After 6-24 hours, 1000 vermiform eggs or 1000 J2 Meloidogyne incognita (RKN) are inoculated into each pot. Watering of the test plants is then restricted to only water as needed to prevent wilt for a period of 24 hours. After the 24 hour restricted watering, normal sub-irrigation watering is done for the duration of the test. Cucumber plants are harvested approximately 14 days after inoculation by washing sand off the roots. A root gall rating and visual phytotoxicity rating is assigned using the following scales: Gall rating scale (Gall: % root mass galled): 0=0-5%; 1=6-20%; 2=21-50%; and 3=51-100%. Visual phytotoxicity scale is also assigned (Vis. tox; visual reduction in root mass compared to the control): rs1=mild stunting; rs2=moderate stunting; rs3=severe stunting.

Experiments in soybeans using soy cyst nematodes (SCN) are similar to the cucumber RKN assay except for the following changes. Soybean seeds are planted in 100% sand in two inch square plastic pots. The oligonucleotide solution is applied when the soybeans show the first trifoliate beginning to emerge, about 10 to 12 days after planting. At least six hours after application of the oligonucleotide solution, the nematode soybean cyst nematode (SCN) inoculum (1000 vermiform eggs or 1000 J2s) is applied to the pots. Watering of the test plants is then restricted to only water as needed to prevent wilt for a period of 24 hours. After the 24 hour restricted watering, normal sub-irrigation watering is done for the duration of the test. Twenty eight days after inoculation the plants are harvested and cysts counted

Experiments in corn using lesion nematodes are similar to above except for the following changes. Corn plants growing in a sand:Turface mix 2:1 in 4 inch deep pots (Turface™ MVP, Profile Products, LLC., Buffalo Grove, Ill.). Treatment with oligonucleotide solution is done when the plants are approximately 8-10 old. At least six hours after inoculation of the oligonucleotide solution, plants are inoculated with 2 gm of P. scribneri infested corn roots which are then removed from the pot after 7 days. Watering of the test plants is then restricted to only water as needed to prevent wilt for a period of 24 hours after inoculation. After the 24 hour restricted watering, normal sub-irrigation watering as needed is done for the duration of the test. 12-14 days post inoculation, plants are harvested and nematodes extracted for 6 days from the cut up roots in a mist tent.

### Application of Oligonucleotides to Seeds for Nematode Control

Cucumber seeds are soaked approximately 5-72 hours in nucleotides, either ssDNA and/or dsRNA oligos directed to the promoter and/or targeting the coding region of a target of interest. Seeds can also be soaked in water for a few hours prior to soaking in oligonucleotide solution. Soaking solution consists of 20 nm of each ssDNA nucleotide or 0.03-1 nm dsRNA, 0.1% silwet L77, 50 mM NaPO4 in a final volume 200 uL in water. The radicals of the cucumber seeds emerge within 72 hours, after which the seeds are placed on germination paper until root length is approximately 2 inches. Seedlings are transplanted to sand vials for RKN inoculation 24 hours later. Ten mL dry sand is added to each vial and seedlings are planted by tilting the vial and laying the seedling in the correct orientation so that the cotyledons are just above the sand and then tilting back to cover the radicles with sand. 3.3 ml water is added to each vial and the vials placed in racks under fluorescent light banks. 500 vermiform eggs or 300 J2 RKN are inoculated in each tube in 50 uL of deionized or spring water. Harvest of the cucumber plants is performed 10 to 12 days after inoculation by washing sand off the roots. A root gall rating and visual phytotoxicity rating is assigned using the following scales: Gall rating scale (Gall: % root mass galled): 0=0-5%; 1=6-20%; 2=21-50%; and 3=51-100%. The average of the triplicate gall rating is then calculated: green=0.00-0.33 (no galls); yellow=0.67-1.33 (mild galling); orange=1.67-2.33 (moderate galling); red=2.67-3.00 (severe galling). Visual phytotoxicity scale is also assigned (Vis. tox; visual reduction in root mass compared to the control): rs1=mild stunting; rs2=moderate stunting; rs3=severe stunting.

Experiments in soybeans using soy cyst nematodes (SCN) are similar to RKN assays except for the following changes. After 5-72 hours of soaking soybean seeds are planted in 100% sand in two inch square plastic pots. Seeds can also be soaked in water for a few hours prior to soaking in oligonucleotide solution. Seven days after planting the soybean seed, the nematode soybean cyst nematode (SCN) inoculum (1000 vermiform eggs or 1000 J2s) are applied to the pot. Watering of the test plants is then restricted to only water as needed to prevent wilt for a period of 24 hours. After the 24 hour restricted watering, normal sub-irrigation watering is done for the duration of the test. Twenty eight days after inoculation the test is harvested and cysts counted.

Experiments in corn using lesion nematodes are similar to above except for the following changes. After 5-72 hours of soaking corn seeds are planted in a sand:Turface mix 2:1 in 4 inch deep pots (Turface™ MVP, Profile Products, LLC., Buffalo Grove, Ill.). Seeds can also be soaked in water for a few hours prior to soaking in oligonucleotide solution. Inoculum of 2 gm of roots P. scribneri infested corn roots are applied at seeding and removed from the pot after 7 days. Watering of the test plants is then restricted to only water as needed to prevent wilt for a period of 24 hours after inoculation. After the 24 hour restricted watering, normal sub-irrigation watering as needed is done for the duration of the test. 12-14 days post inoculation, plants are harvested and nematodes extracted for 6 days from the cut up roots in a mist tent.

RKN and SCN J2s are prepared from hatchbowls using the following solutions: RKN solution: 1 L aerated tap water, 1 ml of 50 mg/ml kanamycin, 0.5 ml of 20 mg/ml imazalil sulfate; SCN solution: 1 L aerated tap water, 1 ml of 50 mg/ml kanamycin, 0.5 ml of 20 mg/ml imazalil sulfate, 1430 mg zinc sulfate.

Hatchbowls are autoclaved 6 oz bowls, lined with screen mesh and paper filter. Approximately 20 ml of appropriate hatch solution is poured into each bowl. Eggs are then place in the bowls and covered with foil. The bowls are then placed in a 25° C. incubator overnight. The next day the hatched J2's are extracted, additional solution added as needed and replaced in the incubator. Each bowl is used for 2 weeks and then disposed.

### Example 7. Protection of Soybean from Root Knot Nematodes (RKN)

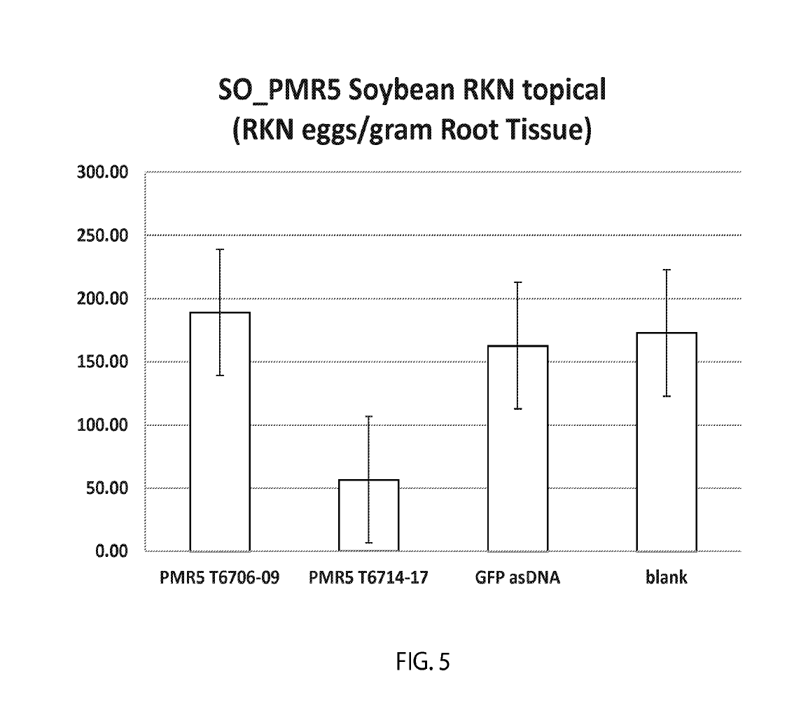

Soybean variety W2 at the unifoliate stage were contacted with the indicated control and test solutions (Table 18) and inoculated with 2500 Meloidogyne incognita eggs 24 hrs later. The indicated amounts of oligomers were provided in 5 mM NaPO₄, 1% Ammonium Sulfate, and 0.25% Silwet™ (wt percent). Approximately 50 μl of solution containing the ssDNA oligonucleotides of Table 19, in pools of 4 ssDNAs/pool, was applied to each plant and 4 plants were subjected to each treatment. Approximately 2500RKN (M. incognita) were inoculated into the soil about 1 day post treatment. Root weights and egg counts were recorded approximately 25 days post infection (Table 20). RKN infection was analyzed by comparing the number of eggs produced per gram of root tissue for each group as shown in Table 21 and FIG. 5. ANOVA analysis of the Table 21 and FIG. 5 egg/gram root data is provided in Table 22.

TABLE 18
Treatments

| | | Oligos |
|---|---|---|
| Trtmnt # | Description: | Final Conc. |
| 1 | PMR5 T6706-09 | 0.4 nmol/ul (each) |
| | (SEQ ID NO: 12, 13, 14, 15) | 1.6 nmol/ul (total) |
| 2 | PMR5 T6714-17 | 0.4 nmol/ul (each) |
| | (SEQ ID NO: 16, 17, 18, 19) | 1.6 nmol/ul (total) |
| 3 | GFP asDNA | 1.6 nmol/ul (total) |
| | (SEQ ID NO: 20) | |
| 4 | blank | 0 nmol/ul |

TABLE 19
Oligonucleotides used

| | | Location | |
|---|---|---|---|
| | | in Soybean | |
| | | PMR5 | SEQ |
| | | Gene (SEQ | ID |
| Oligo | Sequence | ID NO: 8) | NO |
| T6706 | TGTATCTGAGGTAATCAGAATCAGG | 259_283 | 12 |
| T6707 | GAGGTCACAGTTGAGGGGTCTCCAT | 285_309 | 13 |
| T6708 | AGAAACTCCACCCCATTGAACCTAG | 311_335 | 14 |
| T6709 | CATCACAGTTTTGCCCTTCATTTGC | 339_363 | 15 |
| T6714 | TGAAGAGAGCCTTGATGATCCCACC | 638_662 | 16 |
| T6715 | CCTCCTAATTCCATATAATCCCACC | 665_689 | 17 |
| T6716 | AGTTTCACCATAGCAGTTCTTTTGTA | 864_888 | 18 |
| T6717 | GCTGTGCCAGTGCTAGTAATTGGAG | 890_914 | 19 |

TABLE 20
Summary of Root Weight Data

| | Root | Root | Root | Root | AVG | |
|---|---|---|---|---|---|---|
| | Wt | Wt | Wt | Wt | Root | |
| | grams | grams | grams | grams | Wt | |
| Treatment | REP1 | REP2 | REP3 | REP4 | grams | stdev |
| 1 (PMR5 | 13.3 | 13.8 | 11.7 | 15.3 | 13.30 | 1.81 |
| T6706-09) | | | | | | |
| 2 (PMR5 | 13.7 | 11.1 | 8.7 | 12.2 | 11.10 | 1.79 |
| T6714-17) | | | | | | |
| 3 (GFP asDNA) | 13 | 14.2 | 9.3 | 14.1 | 14.20 | 2.80 |
| 4 (blank) | 13 | 13 | 11.5 | 11.1 | 13.00 | 1.00 |

TABLE 21
Eggs per Gram of Root

| | | | | Average | |
|---|---|---|---|---|---|
| | Oligos | | | Eggs/gm | |
| Treatment # | used | Count | Sum | Root | Variance |
| 1 | PMR5 | 4 | 756.1 | 189.025 | 13398.76 |
| | T6706-09 | | | | |
| | (SEQ ID | | | | |
| | NO: 12, 13, | | | | |
| | 14, 15) | | | | |
| 2 | PMR5 | 4 | 226.5 | 56.625 | 697.9825 |
| | T6714-17 | | | | |
| | (SEQ ID | | | | |
| | NO: 16, 17, | | | | |
| | 18, 19) | | | | |
| 3 | GFP asDNA | 4 | 651 | 162.75 | 2425.937 |
| | (SEQ ID | | | | |
| | NO: 20) | | | | |
| 4 | none | 4 | 691.3 | 172.825 | 6078.223 |

TABLE 22
ANOVA Analysis of Egg/gram Root Data

| Source of | | | | | | |
|---|---|---|---|---|---|---|
| Variation | SS | df | MS | F | P-value | F crit |
| Between | 43349.04 | 3 | 14449.68 | 2.557363 | 0.104065 | 3.490295 |
| Groups | | | | | | |
| Within Groups | 67802.71 | 12 | 5650.226 | | | |
| Total | 111151.7 | 15 | | | | |
| | | df = 1.782 | | | | |

std of diff = 53.15

lsd = 94.72

½lsd = 47.4

### Example 8. Resistance of Cucumber Seedlings to Root Knot Nematodes

Cucumber seeds (Straight Eight, Burpee Seeds, Warminster, Pa., USA) at 4 seeds/well were soaked in a 24 well plate (flat bottom). Soak solution of 200 uL contained Ultrapure™ Gibco water, 20 mM NaPO₄ buffer and nucleotides (either ssDNA (highest conc—80 nmol) and/or dsRNA (highest conc—0.15 nmol) oligos) for approximately 72 hours on a rockerat room temperature (˜25° C.). Surfactant was omitted in this assay. Controls included a negative control trigger molecule (eg. GFP or degenerate oligos that do not target endogenous cucumber sequences) and buffer alone. The radicals of the cucumber seeds emerged within 72 hours and the seeds were then placed on germination pouches (Cyg™ Seed Germination Pouches, Mega International, Saint Paul, Minn.; on the internet at mega-international.com/tech.htm) with 20 mL of tap water for three days at 25° C. with a 12 hr light cycle. Roots were approximately 2 inches in length and observations about effect of treatment on radicals could be assessed at this time. Seedlings were transplanted to sand vials after three days. The “QuickSand” assay was performed by adding 10 mL dry sand to each glass flat bottom vial, planting seedlings by tilting the vial, laying the seedling in an orientation so that the cotyledons are just above the sand and then tilting back to cover the radicals with sand. About 3.5 ml water was added to each vial and the vials placed in racks under fluorescent light banks at room temperature. After three days, 250 Meloidogyne incognita J2 were inoculated in each tube in 50 to 200 uL of aerated tap water. Harvest of the cucumber plants was performed 10 to 11 days after inoculation by washing sand off the roots. A root gall rating and visual phytotoxicity rating was assigned using the following scales: Gall rating scale (Gall: % root mass galled): 0=0-5%; 1=6-20%; 2=21-50%; and 3=51-100%. Visual phytotoxicity scale was also assigned (Vis. tox; visual reduction in root mass compared to the control): rs1=mild stunting; rs2=moderate stunting; rs3=severe stunting. The following calculations are then done to determine efficacy: average of at least three replicates, standard deviation of four replicates, % reduction compared to control, single factor ANOVA test, ½ LSD value for data.

Pools of the dsRNAs T6860 (SEQ ID NO:53), T6861 (SEQ ID NO:54), T6862 (SEQ ID NO:55), and T6863 (SEQ ID NO:56) were tested for RKN control in the aforementioned cucumber seedling assay at 0.06 nmol/each dsRNA and 0.15 nmol/each dsRNA (Table 23).

TABLE 23
Control of RKN by PMR5 dsRNA pools

| | | | | | | | | | | % |
|---|---|---|---|---|---|---|---|---|---|---|
| Treatment | Rep Score | | | | | | | AVG | stdev | Reduction |
| PMR5 Pool | 30 | 35 | 35 | 25 | 35 | | | 32.00 | 4.47 | 30.0 |
| T6860-63 | | | | | | | | | | |
| coding | | | | | | | | | | |
| (0.06 nmol | | | | | | | | | | |
| total) | | | | | | | | | | |
| PMR5 | 25 | 40 | 20 | | | | | 28.33 | 10.41 | 38.0 |
| T6860-63 | | | | | | | | | | |
| coding | | | | | | | | | | |
| (0.15 nmol | | | | | | | | | | |
| total) | | | | | | | | | | |
| GFP | 40 | 60 | 47.5 | | 35 | 45 | 47.5 | 45.83 | 8.47 | −0.3 |
| control | | | | | | | | | | |
| (0.15 nmol | | | | | | | | | | |
| total)) | | | | | | | | | | |
| No dsRNA | 47.5 | 42.5 | 55 | 42.5 | 40 | 47.5 | 45 | 45.71 | 4.94 | 0.0 |

The Anova: Single factor analysis is provided in Tables 24, 25, and 26.

TABLE 24
Anova: Summary.

| Groups | Treatment | Count | Sum | Average | Variance |
|---|---|---|---|---|---|
| Row 1 | PMR5 | 2 | 36.47214 | 18.23607 | 378.8916 |
| | T6860-63 | | | | |
| | coding | | | | |
| | (0.06 nmol | | | | |
| | total) | | | | |
| Row 2 | PMR5 | 2 | 38.74166 | 19.37083 | 160.6529 |
| | T6860-63 | | | | |
| | coding | | | | |
| | (0.15 nmol | | | | |
| | total) | | | | |
| Row 3 | GFP | 2 | 54.29895 | 27.14948 | 698.1731 |
| | control | | | | |
| | (0.15 nmol | | | | |
| | total)) | | | | |
| Row 4 | No | 2 | 50.6544 | 25.3272 | 831.2664 |
| | dsRNA | | | | |

TABLE 25
ANOVA

| Source | | | | | | |
|---|---|---|---|---|---|---|
| of | | | | | | |
| Vari- | | | | | | |
| ation | SS | df | MS | F | P-value | F crit |
| Be- | 115.1635 | 3 | 38.38783 | 0.074216 | 0.970689 | 6.591382 |
| tween | | | | | | |
| Groups | | | | | | |
| Within | 2068.984 | 4 | 517.246 | | | |
| Groups | | | | | | |
| Total | 2184.148 | 7 | | | | |

TABLE 26
ANOVA analysis

| | | |
|---|---|---|
| std of diff | 8.04088 | |
| df | 2.132 | Table |
| LSD | 17.14316 | |
| 1/2 LSD | 8.571578 | |

Individual dsRNA molecules (T6860 (SEQ ID NO:53), T6861 (SEQ ID NO:54), T6862 (SEQ ID NO:55), and T6863 (SEQ ID NO:56) were also tested for RKN control in the aforementioned cucumber seedling assay at 0.06 nmol/each dsRNA and 0.15 nmol/each dsRNA (Table 27).

TABLE 27
Individual dsRNA treatment results.

| | SEQ | | | | | | | | | | | |
|---|---|---|---|---|---|---|---|---|---|---|---|---|
| | ID | | | | | | | | | | | % |
| Treatment | NO | Rep Score | | | | | | | | AVG | stdev | Reduction |
| PMR5 | 53 | | | | | 27.5 | 40 | 22.5 | 35 | 31.25 | 7.77 | 31.6 |
| T6860 | | | | | | | | | | | | |
| (0.06 nmol) | | | | | | | | | | | | |
| PMR5 | 54 | | | | | 27.5 | 20 | 30 | 27.5 | 26.25 | 4.33 | 42.6 |
| T6861 | | | | | | | | | | | | |
| (0.06 nmol) | | | | | | | | | | | | |
| PMR5 | 55 | | | | | 42.5 | 25 | 35 | 30 | 33.13 | 7.47 | 27.5 |
| T6862 | | | | | | | | | | | | |
| (0.06 nmol) | | | | | | | | | | | | |
| PMR5 | 56 | | | | | 40 | 30 | 42.5 | 35 | 36.88 | 5.54 | 19.3 |
| T6863 | | | | | | | | | | | | |
| (0.06 nmol) | | | | | | | | | | | | |
| PMR5 | 53 | | | | | 37.5 | 27.5 | 45 | 20 | 32.50 | 10.99 | 28.9 |
| T6860 | | | | | | | | | | | | |
| (0.15 nmol) | | | | | | | | | | | | |
| PMR5 | 54 | | | | | 40 | 35 | 32.5 | 32.5 | 35.00 | 3.54 | 23.4 |
| T6861 | | | | | | | | | | | | |
| (0.15 nmol) | | | | | | | | | | | | |
| PMR5 | 55 | | | | | 30 | 30 | 40 | 45 | 36.25 | 7.50 | 20.7 |
| T6862 | | | | | | | | | | | | |
| (0.15 nmol) | | | | | | | | | | | | |
| PMR5 | 56 | | | | | 20 | 25 | 25 | 32.5 | 25.63 | 5.15 | 43.9 |
| T6863 | | | | | | | | | | | | |
| (0.15 nmol) | | | | | | | | | | | | |
| GFP control | | 40 | 60 | 47.5 | | 35 | 45 | 47.5 | | 45.83 | 8.47 | −0.3 |
| (0.15 nmol) | | | | | | | | | | | | |
| No dsRNA | | 47.5 | 42.5 | 55 | 42.5 | 40 | 47.5 | 45 | | 45.71 | 4.94 | 0.0 |

### Example 9. Control of Phytophthora Root Rot in Soybean

DNA oligos directed to either promoter (prm) or coding regions (CDS) of the PMR5 gene were tested for their ability to control Phytophthora root rot (PRR) on soybean. Disease development was good as the non-inoculated control roots were 3 fold larger than inoculated only roots that were not treated with oligos. In this test, all plants were fertilized with nitrogen by sub-irrigation one time before inoculation and were not chlorotic. In treatment 14, a small amount of fertilizer was also added directly to the pot before inoculation.

Root rate loss was calculated by the following:

non-inoculated root weight−treatment root weight=root weight loss.

Pool T6702-T6705 had statistically less root loss than the formulation blank, indicating that one or more of the oligos in that pool conferred control of Phytophtora Root Rot in soybean.

TABLE 28
Percentage reduction in root weight loss

| | | | | | % reduction in | |
|---|---|---|---|---|---|---|
| | | SEQ ID | root | root wt | root wt loss | |
| Trt# | Oligo Pool | NO: | wt | loss | FB | FC |
| 1 | as T6686-T6689 | 57-60 | 2.3 | 5.1 | −7.4 | −17.4 |
| | prm | | | | | |
| 2 | as T6690-T6693 | 61-64 | 3.2 | 4.1 | 12.2 | 4.1 |
| | prm | | | | | |
| 3 | as T6694-T6697 | 65-68 | 3.0 | 4.3 | 8.5 | 0.0 |
| | prm | | | | | |
| 4 | as T6698-T6701 prm | 69-72 | 2.3 | 5.0 | −6.0 | −15.9 |
| 5 | as T6702-T6705 | 73-76 | 3.6 | 3.7 | 20.7 | 13.4 |
| | CDS | | | | | |
| 6 | as T6706-T6709 | 77-80 | 2.5 | 4.8 | −1.6 | −11.0 |
| | CDS | | | | | |
| 7 | as T6710-T6713 | 81-84 | 2.7 | 4.6 | 1.6 | −7.6 |
| | CDS | | | | | |
| 8 | as T6714-T6717 | 85-88 | 3.0 | 4.3 | 8.0 | −0.6 |
| | CDS | | | | | |
| 9 | as T6718-T6721 | 89-92 | 2.5 | 4.9 | −3.2 | −12.8 |
| | CDS | | | | | |
| 10 | SEQ ID NO 20 | | 3.0 | 4.3 | 8.0 | 0.0 |
| 11 | formulation blank | | 2.6 | 4.7 | 0.0 | −9.9 |
| 12 | inoculation only | | 2.3 | 5.0 | | |
| 13 | not inoculated | | 7.3 | 0.0 | | |

Legend:
prm = promoter;
CDS = coding sequence

TABLE 29
Percentage root weight increase

| | | | ROOT | % root | |
|---|---|---|---|---|---|
| | | SEQ | Weight | wt increase | |
| Trt# | | ID NO: | average | FB | FC |
| 1 | as T6686-89 prm | 57-60 | 2.3 | −13.5 | −25.0 |
| 2 | as T6690-93 prm | 61-64 | 3.2 | 22.1 | 5.8 |
| 3 | as T6694-97 prm | 65-68 | 3.0 | 15.4 | 0.0 |
| 4 | as T6698-701 prm | 69-72 | 2.3 | −10.9 | −22.8 |
| 5 | as T6702-05 CDS | 73-76 | 3.6 | 37.5 | 19.2 |
| 6 | as T6706-09 CDS | 77-80 | 2.5 | −2.9 | −15.8 |
| 7 | as T6710-13 CDS | 81-84 | 2.7 | 2.9 | −10.8 |
| 8 | as T6714-17 CDS | 85-88 | 3.0 | 14.4 | −0.8 |
| 9 | as T6718-21 CDS | 89-92 | 2.5 | −5.8 | −18.3 |
| 10 | SEQ ID NO 20 | | 3.0 | 14.4 | −0.8 |
| 11 | formulation blank | | 2.6 | −1.0 | −14.2 |
| 12 | inoculation only | | 2.3 | 3.2 | |
| 13 | not inoculated | | 7.3 | 3X larger | |
| | | | | than trt 12 | |
| | | | | or trt 14 | |
| 14 | fertilized and | | 1.3 | 5.5 | |
| | inoculated | | | | |

### Example 10. Use of VIGS to Select Polynucleotides that Suppress Expression of PMR5 Genes

To select candidate polynucleotides that can potentially suppress endogenous PMR5 genes, polynucleotide sequences are introduced into a Tomato Golden Mosaic Virus (ToGMV) vector and tested for their ability to provide Virus-Induced Gene Silencing of the endogenous PMR5 gene in plants. Polynucleotide sequences that exhibit VIGS-mediated suppression of PMR5 are subsequently screened for their ability to suppress expression of PMR5 when applied to a plant with a transfer agent.

A modification of the sprout vacuum-infiltration-mediated agroinoculation method for virus-induced gene silencing protocol described in Yan et al. Plant Cell Rep (2012) 31:1713-1722 can be used. Surface sterilized tomato seeds are first germinated on ¼ Murashige-Skoog media plus Cefotaxime. After about 3 days, Agrobacterium component A containing ToGMoV:PMR5 Suppression Sequence and the ToGMoV component B are each separately inoculated into 10 mL Luria Broth with appropriate concentrations of spectinomycin, gentamycin, and chloramphenicol and shaken at 24° C. for about 1-2 days to prepare an Agrobacterium inoculum containing the ToGMoV vector components. The A genome component is known to encode viral functions necessary for viral DNA replication, while the B genome component specifies functions necessary for spread of the virus through the infected plant (Revington, et al. Plant Cell. 1989 October; 1(10): 985-992). After about one to two days of growth, the Agrobacterium are pelleted by centrifugation and resuspended to a final OD600 of 0.05 in Infiltration Buffer (10 mM MES, 10 mM MgCl, 100 uM Acetosyringone). The Agrobacterium A component and B component are mixed for use at a 1:1 ratio and an Infiltration buffer only control (Mock) is also prepared. The A and B component mixture and the mock Infiltration buffer control are then allowed to incubate at room temperature (˜25° C.) for 3-4 hours. About 3 mls of each sample (i.e. ToGMV vector with a given test PMR5 suppression sequence) is transferred into a small microtiter dish. Typically, 1 microtiter plate (6-24 wells) is used for each test ToGMV vector with a given test PMR5 suppression sequence and 1 microtiter plate is used for the mock control (Infiltration buffer only). About 3-5 sprouts are added to each well, a vacuum is pulled for 10 seconds and then stopped. Pulling and stopping of the vacuum is then repeated 2 more times. Vacuum infiltrated sprouts are planted in soil, taking care not to cross contaminate samples. This can be accomplished by changing gloves and using new tweezers. Planted sprouts are covered with humidity dome and left at room temperature (˜25° C.) overnight to recover. After a day, potted sprouts are transferred to a growth chamber. Phenotypes associated with PMR5 suppression (i.e. fungal and/or nematode resistance) can be observed by challenging potted plants that were agroinfected with a ToGMV vector containing a sequence that provides for suppression of the endogenous PMR5 gene but is not observed in plants subjected to the mock control (Infiltration buffer only) or a ToGMV vector containing a sequence that does not provide for suppression of the endogenous PMR5 gene.

1. A method for producing a soybean plant exhibiting an improvement in fungal and/or nematode disease resistance comprising topically applying to a soybean plant surface a composition that comprises:
a. at least one polynucleotide of 18 to about 95 nucleotides in length, wherein the polynucleotide comprises a segment of 18 contiguous nucleotides that are essentially identical or essentially complementary to a PMR5 gene or to a transcript of said gene; wherein said polynucleotide is not operably linked to promoter or a viral vector and wherein said polynucleotide is not integrated into the plant chromosome; and
b. a transfer agent, wherein said soybean plant exhibits an improvement in fungal and/or nematode disease resistance that results from suppression of a soybean PMR5 gene.

2. The method of claim 1, wherein said polynucleotide molecule comprises sense ssDNA, sense ssRNA, dsRNA, dsDNA, a double stranded DNA/RNA hybrid, anti-sense ssDNA, or anti-sense ssRNA.

3. The method of claim 1, wherein said polynucleotide is 25 nucleotides in length.

4. The method of claim 3, wherein the gene or the transcript is a soybean PMR5 gene or transcript, and the polynucleotide molecule is selected from the group consisting of SEQ ID NO: 12-19, 57-127, and SEQ ID NO: 128, or the polynucleotide comprises a segment of 18 contiguous nucleotides that are essentially identical or essentially complementary to SEQ ID NO: 8 or 11.

5. The method of claim 1, wherein said composition comprises any combination of two or more of said polynucleotide molecules.

6. (canceled)

7. The method of claim 1, wherein said composition further comprises a non-polynucleotide herbicidal molecule, a polynucleotide herbicidal molecule, a polynucleotide that suppresses an herbicide target gene, an insecticide, a fungicide, a nematocide, or a combination thereof.

8. The method of claim 1, wherein said composition further comprises a non-polynucleotide herbicidal molecule and said plant is resistant to said herbicidal molecule.

9. The method of claim 1, wherein said transfer agent comprises an organosilicone preparation.

10-11. (canceled)

12. A soybean plant obtained by the method of claim 1, wherein said soybean plant comprises an exogenous polynucleotide of 18 to 95 nucleotides in length, wherein the polynucleotide comprises a segment of 18 continuous nucleotides that are identical or complementary to a PMR5 gene or to a transcript of said gene, wherein said endogenous polynucleotide is not operably linked to a promoter or to a viral vector, is not integrated into the chromosomal DNA of the plant, does not result from transcription of the exogenous polynucleotide from DNA integrated into a chromosome of the plant, and is not found in a non-transgenic plant; and
wherein said plant exhibits an improvement in fungal disease resistance that results from suppression of a soybean PMR5 gene.

13. The plant of claim 12, wherein a progeny plant or a plant part derived therefrom exhibits an improvement in fungal and/or nematode disease resistance.

14. A progeny plant of said plant of claim 12, wherein said progeny plant exhibits said improvement in fungal and/or nematode disease resistance.

15. A seed of said plant of claim 12, wherein said seed exhibits said improvement in fungal and/or nematode disease resistance.

16. A processed product of said plant of claim 12, wherein said processed product comprises the exogenous polynucleotide of 18 to 95 nucleotides in length comprising a segment of 18 continuous nucleotides that are identical or complementary to a PMR5 gene or to a transcript of said gene and exhibits an improved attribute relative to a processed product of an untreated control plant and wherein said improved attribute results from said fungal and/or nematode disease resistance.

17. A processed product of said progeny plant of claim 14, wherein said processed product exhibits an improved attribute relative to a processed product of an untreated control plant and wherein said improved attribute results from said fungal and/or nematode disease resistance.

18. A processed product of said seed of claim 15, wherein said processed product exhibits an improved attribute relative to a processed product of an untreated control plant and wherein said improved attribute results from said fungal and/or nematode disease resistance.

19. A composition comprising a polynucleotide molecule of 18 to about 95 nucleotides in length, wherein the polynucleotide comprises a segment of 18 contiguous nucleotides that are essentially identical or essentially complementary to a PMR5 gene or transcript of said gene, wherein said polynucleotide is not operably linked to a promoter, wherein said polynucleotide is not operably linked to a viral vector and wherein said polynucleotide is not integrated into the plant chromosome and a transfer agent.

20. The composition of claim 19, wherein said polynucleotide is 25 nucleotides in length.

21. The composition of claim 19, wherein the gene or the transcript is a soybean PMR5 gene or transcript, and the polynucleotide molecule is selected from the group consisting of SEQ ID NO: 12-19, 57-127, and SEQ ID NO: 128, or the polynucleotide comprises at least 18 contiguous nucleotides that are essentially identical or essentially complementary to SEQ ID NO: 8 or 11.

22. (canceled)

23. The composition of claim 19, wherein said composition further comprises a non-polynucleotide herbicidal molecule, a polynucleotide herbicidal molecule, a polynucleotide that suppresses an herbicide target gene, an insecticide, a fungicide, a nematocide, or a combination thereof.

24. The composition of claim 19, wherein said transfer agent is an organosilicone preparation.

25. The composition of claim 19, wherein said polynucleotide is not physically bound to a biolistic particle.

26-46. (canceled)
